(19) United States
(12) Patent Application Publication
(10) Pub. No.: US 2016/0348035 A1
(43) Pub. Date: Dec. 1, 2016

(54) Compositions and Methods for Improving Properties of Non-Cellulosic Textile Materials with Xyloglucan Endotransglycosylase

(71) Applicant: NOVOZYMES A/S, Bagsvaerd (DK)

(72) Inventors: Alex Berlin, Davis, CA (US); Romil Benyamino, Sacramento, CA (US); Jason Quinlan, Albany, CA (US)

(73) Assignee: Novozymes A/S, Bagsvaerd (DK)

(21) Appl. No.: 15/112,452

(22) PCT Filed: Mar. 5, 2015
(86) PCT No.: PCT/US2015/018932

§ 371 Date: Jul. 19, 2016

Related U.S. Application Data
(60) Provisional application No. 61/948,245, filed on Mar. 5, 2014.

(51) Int. Cl. C11D 3/386; C12N 9/10; D06M 16/00; C11D 3/22; D06M 15/03

(52) U.S. Cl. CPC C11D 3/38636; C11D 3/222; D06M 15/03; D06M 16/003; C12N 9/1051; C12Y 204/01207; D06M 2101/20

(57) ABSTRACT

The present invention relates to methods for modifying a non-cellulosic textile material comprising treating the non-cellulosic textile material with a composition comprising a xyloglucan endotransglycosylase and (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer, under conditions leading to a modified non-cellulosic textile material, where the modified non-cellulosic textile material possesses a textile improvement compared to the unmodified non-cellulosic textile material. The present invention also relates to modified non-cellulosic textile materials with a textile improvement.

### REFERENCE TO A SEQUENCE LISTING

This application contains a Sequence Listing in computer readable form, which is incorporated herein by reference.

### BACKGROUND OF THE INVENTION

Field of the Invention

The present invention relates to compositions and methods for improving properties of non-cellulose textile materials with xyloglucan endotransglycosylase.

Description of the Related Art

Xyloglucan endotransglycosylase (XET) is an enzyme that catalyzes endotransglycosylation of xyloglucan, a structural polysaccharide of plant cell walls. The enzyme is present in most plants, and in particular, land plants. XET has been extracted from dicotyledons and monocotyledons.

Xyloglucan is present in cotton, paper, or wood fibers (Hayashi et al., 1988, Carbohydrate Research 181: 273-277) making strong hydrogen bonds to cellulose (Carpita and Gibeaut, 1993, The Plant Journal 3: 1-30). Adding xyloglucan endotransglycosylase to various cellulosic materials containing xyloglucan alters the xyloglucan mediated interlinkages between cellulosic fibers improving their strength and/or shape-retention and/or anti-wrinkling properties of the cellulosic materials, and maintaining the cellulose-structure while permitting the cellulose fibers to move relative to one another under force.

WO 97/23683 discloses a process for providing a cellulosic material, such as a fabric or a paper and pulp product, with improved strength and/or shape-retention and/or anti-wrinkling properties by using xyloglucan endotransglycosylase. WO 01/07556 discloses laundry and/or fabric and/or color care compositions containing xyloglucan endotransglycosylase in combination with a polysaccharide and/or oligosaccharide to refurbish and/or restore improved tensile strength, enhanced anti-wrinkle, anti-bobbling and anti-shrinkage properties to cellulosic fabrics.

There is a need in the art to improve the properties of non-cellulosic textile materials. Non-limiting examples of such properties include anti-pilling, anti-shrinkage, anti-wear, anti-wrinkle, color appearance, fabric softness, shape retention, static control, flame and chemical resistance, odor control or anti-odor, anti-UV, water-repellency, anti-microbial properties, improved hand or texture, resistance to chemical, biological, radiological or physical hazard, improved association with cellulosic textile materials in textile blends, and/or tensile strength properties.

The present invention provides compositions and methods for improving the properties of non-cellulose textile materials.

### SUMMARY OF THE INVENTION

The present invention relates to methods for modifying a non-cellulosic textile material comprising treating the non-cellulosic textile material with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; and (h) a composition comprising a xyloglucan endotransglycosylase and a xyloglucan oligomer, under conditions leading to a modified non-cellulosic textile material, wherein the modified non-cellulosic textile material possesses a textile improvement compared to the unmodified non-cellulosic textile material.

The present invention also relates to modified non-cellulosic textile materials obtained by such methods.

The present invention also relates to modified non-cellulosic textile materials comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group and a xyloglucan oligomer; (d) a polymeric xyloglucan and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

The present invention also relates to a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; or (h) a composition comprising a xyloglucan endotransglycosylase and a xyloglucan oligomer.

The present invention also relates to detergent or fabric care compositions for non-cellulose textile materials comprising a surfactant and (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The present invention also relates to detergent additives for non-cellulose textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The present invention further relates to textile coatings or finishings for non-cellulose textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

### BRIEF DESCRIPTION OF THE FIGURES

FIG. 1 shows a restriction map of pDLHD0012.

FIG. 2 shows a restriction map of pMMar27.

FIG. 3 shows a restriction map of pEvFz1.

FIG. 4 shows a restriction map of pDLHD0006.

FIG. 5 shows a restriction map of pDLHD0039.

FIG. 6 shows the fluorescence intensity of the solution phase of fluorescein isothiocyanate-labeled xyloglucan (FITC-XG) incubated with filter paper, incubated with filter paper in the presence of Vigna angularis xyloglucan endotransglycosylase 16 (VaXET16), or incubated with no filter paper.

FIG. 7 shows enhancement of polyethylene terephthalate-xyloglucan binding by VaXET16 and Arabidopsis thaliana xyloglucan endotransglycosylase 14 (AtXET14).

FIG. 8 shows enhancement of polyethylene terephthalate (PET)-xyloglucan binding by VaXET16 observed by laser scanning confocal microscopy. FIG. 8A shows the confocal microscopy image of PET incubated in the absence of FITC-XG at 10× magnification. FIG. 8B shows the confocal microscopy image of PET incubated with FITC-XG at 10× magnification. FIG. 8C shows the confocal microscopy image of PET incubated in the presence of both FITC-XG and VaXET16 at 10× magnification.

FIG. 9 shows enhancement of cellulose and polyethylene terephthalate binding of xyloglucan of various average molecular weights by VaXET16 as measured by fluorescence intensity and fluorescence polarization.

FIG. 10 shows a chromatogram of various expected molecular weight average FITC-XGs.

FIG. 11 shows the molar ratio of reducing sugar to fluorophore for pooled chromatography fractions.

FIG. 12 shows the fraction of each theoretical FITC-XG bound to PET under the experimental binding conditions with and without VaXET16.

FIG. 13 shows the fold-enhancement of the fraction of FITC-XG bound to PET when VaXET16 was present over the fraction of FITC-XG bound when VaXET16 was absent.

FIG. 14 shows a SDS-PAGE gel of the supernatants of PET-binding reactions sampled at various times.

FIG. 15 shows the effects of protease digestion of VaXET16 and xyloglucanase digestion of xyloglucan or FITC-XG on the release of FITC-XG fluorescence from polyethylene terephthalate as a function of time.

FIG. 16 shows the fluorescence intensity in solution for a FITC-XG solution incubated with nylon.

FIG. 17 shows overlaid transmission and fluorescence confocal microscopy images (on left) and fluorescence emission images (on right) of nylon incubated without FITC-XG (top panels), with FITC-XG and then extensively washed (middle panels), or with FITC-XG and xyloglucan endotransglycosylase solution (bottom panels).

FIG. 18 shows the effect of heat-inactivated VaXET16 on the enhancement of XG-PET binding. The fluorescence intensities of supernatants of the various incubations were measured at time 0 (white); 90 hours of incubation (gray); and 48 hours after addition of CELLIC CTec® or additional VaXET16 (hatched). The enzyme or enzyme composition that was added at 90 hours is indicated in parentheses.

FIG. 19 shows the fluorescence image of various test fabrics treated with fluorescently-labeled silicon dioxide (FITC-silica) in the presence or absence of VaXET16.

FIG. 20 shows the mean intensity of various test fabrics treated with FITC-silica in the presence or absence of VaXET16 prior to washing in detergent.

FIG. 21 shows the fluorescence image of various test fabrics treated with FITC-silica in the presence or absence of VaXET16 following washing with a standard laundry detergent.

FIG. 22 shows the mean intensity of various test fabrics treated with FITC-silica in the presence or absence of VaXET16 following washing with a standard laundry detergent.

FIG. 23 shows a fluorescence image of variously incubated multi-fabric strips with FITC-XG in the presence or absence of VaXET16.

### DEFINITIONS

As used herein, the singular forms “a”, “an”, and “the” are intended to include the plural forms as well, unless the context clearly indicates otherwise.

Functionalized xyloglucan oligomer: The term “functionalized xyloglucan oligomer” means a short chain xyloglucan oligosaccharide, including single or multiple repeating units of xyloglucan, which has been modified by incorporating a chemical group. The xyloglucan oligomer is preferably 1 to 3 kDa in molecular weight, corresponding to 1 to 3 repeating xyloglucan units. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The incorporated reactive groups can be derivatized with a compound of interest to directly provide a textile improvement or to coordinate metal cations and/or to bind other chemical entities that interact (e.g., covalently, hydrophobically, electrostatically, etc.) with the reactive groups. The derivatization can be performed directly on a functionalized xyloglucan oligomer comprising a reactive group or after the functionalized xyloglucan oligomer comprising a reactive group is incorporated into polymeric xyloglucan. Alternatively, the xyloglucan oligomer can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group. The terms “functionalized xyloglucan oligomer” and “functionalized xyloglucan oligomer comprising a chemical group” are used interchangedly herein.

Non-cellulosic textile material: The term “non-cellulosic textile material” means any textile material including yarns, yarn intermediates, threads, fibers, non-woven materials, natural materials, synthetic materials, and any other textile material, fabrics made of these materials, and products made from the fabrics (e.g., garments and other articles). The textile may be in the form of knits, wovens, denims, non-wovens, felts, yarns, and towelling. The textile is non-cellulose based such as natural polyamides including wool, camel, cashmere, mohair, rabbit, and silk or synthetic polymers such as nylon, aramid, polyester, acrylic, polypropylene, and spandex/elastane, or blends thereof. The fabric may be conventional washable laundry, for example, stained household laundry. When the term fabric or garment is used it is intended to include the broader term textiles as well.

Polymeric xyloglucan: The term “polymeric xyloglucan” means short, intermediate or long chain xyloglucan oligosaccharide or polysaccharide encompassing more than one repeating unit of xyloglucan, e.g., multiple repeating units of xyloglucan. Most optimally, polymeric xyloglucan encompasses xyloglucan of 50-200 kDa number average molecular weight, corresponding to 50-200 repeating units. A repeating motif of xyloglucan is composed of a backbone of four beta-(1-4)-D-glucopyranose residues, three of which have a single alpha-D-xylopyranose residue attached at O-6. Some of the xylose residues are beta-D-galactopyranosylated at O-2, and some of the galactose residues are alpha-L-fucopyranosylated at O-2. The term “xyloglucan” herein is understood to mean polymeric xyloglucan.

Polymeric xyloglucan functionalized with a chemical group: The term “polymeric xyloglucan functionalized with a chemical group” means a polymeric xyloglucan that has been modified by incorporating a chemical group. The polymeric xyloglucan is short, intermediate or long chain xyloglucan oligosaccharide or polysaccharide encompassing more than one repeating unit of xyloglucan, e.g., multiple repeating units of xyloglucan. The polymeric xyloglucan encompasses xyloglucan of 50-200 kDa number average molecular weight, corresponding to 50-200 repeating units. A repeating motif of xyloglucan is composed of a backbone of four beta-(1-4)-D-glucopyranose residues, three of which have a single alpha-D-xylopyranose residue attached at O-6. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The chemical group can be incorporated into a polymeric xyloglucan by reacting the polymeric xyloglucan with a functionalized xyloglucan oligomer in the presence of xyloglucan endotransglycosylase. The incorporated reactive groups can then be derivatized with a compound of interest. The derivatization can be performed directly on a functionalized polymeric xyloglucan comprising a reactive group or after a functionalized xyloglucan oligomer comprising a reactive group is incorporated into a polymeric xyloglucan. Alternatively, the polymeric xyloglucan can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group.

Textile improvement: The term “textile improvement” means a benefit not directly related to catalytic stain removal or prevention of redeposition of soils. Examples of such benefits are anti-backstaining, anti-pilling, anti-shrinkage, anti-wear, anti-wrinkle, improved color appearance, fabric softness, improved shape retention, flame or chemical resistance, anti-odor, anti-UV, water-repellency, anti-microbial, improved association between non-cellulosic and cellulosic textiles, improved static control, improved hand or texture, resistance to chemical, biological, radiological or physical hazard, and/or improved tensile strength. Prevention or reduction of dye transfer from one textile to another textile or another part of the same textile is termed anti-backstaining (also termed dye transfer inhibition). Removal of protruding or broken fibers from a textile surface to decrease pilling tendencies or remove already existing pills or fuzz is termed anti-pilling. Coating or reincorporation or smoothing of protruding or broken fibers is also termed anti-pilling. Prevention of or reduction of a decrease in dimensional size is termed anti-shrinkage. Prevention of or repair of abrasion is termed anti-wear. Prevention of wrinkles, recovery of textile from wrinkling, smoothness of seams, and/or retention of creases after repeated home laundering is termed anti-wrinkle. Improvement of the textile-softness or reduction of textile stiffness is termed improved fabric softness. Color clarification of a textile, or enhanced colorfastness to laundering, perspiration, light, chlorine and non-chlorine bleach, heat, or light at high temperature is termed improved color appearance. Resistance to dimensional size change or dimensional size change during home laundering is termed improved shape retention. Elevated combustion temperature or resistance to burning or melting at high temperatures is termed flame resistance. Resistance to chemical reactions, solubilization or degradation in the presence of chemical solvents, acid or alkali is termed chemical resistance. Resistance to adsorption or prevention of the retention of odorous compounds, particularly short chain fatty acids or low vapor pressure organic compounds is termed anti-odor. Opacity to and prevention or repair of oxidative damage caused by UV irradiation is termed anti-UV. Decreased retention of water, or resistance to wetting is termed water repellency. Enhanced microbiostatic or microbiocidal properties are termed anti-microbial. An increase in resistance to induced electrostatic charge of a textile, or increase in decay rate of an induced electrostatic charge in a textile is termed improved static control. Resistance to elongation under force or augmentation of breaking force is termed improved tensile strength.

Xyloglucan endotransglycosylase: The term “xyloglucan endotransglycosylase” means a xyloglucan:xyloglucan xyloglucanotransferase (EC 2.4.1.207) that catalyzes cleavage of a β-(1→4) bond in the backbone of a xyloglucan and transfers the xyloglucanyl segment on to O-4 of the non-reducing terminal glucose residue of an acceptor, which can be a xyloglucan or an oligosaccharide of xyloglucan. Xyloglucan endotransglycosylases are also known as xyloglucan endotransglycosylase/hydrolases or endo-xyloglucan transferases. Some xylan endotransglycosylases can possess different activities including xyloglucan and mannan endotransglycosylase activities. For example, xylan endotransglycosylase from ripe papaya fruit can use heteroxylans, such as wheat arabinoxylan, birchwood glucuronoxylan, and others as donor molecules. These xylans can potentially play a similar role as xyloglucan while being much cheaper in cost since they can be extracted, for example, from pulp mill spent liquors and/or future biomass biorefineries.

Xyloglucan endotransglycosylase activity can be assayed by those skilled in the art using any of the following methods. The reduction in the average molecular weight of a xyloglucan polymer when incubated with a molar excess of xyloglucan oligomer in the presence of xyloglucan endotransglycosylase can be determined via liquid chromatography (Sulova et al., 2003, Plant Physiol. Biochem. 41: 431-437) or via ethanol precipitation (Yaanaka et al., 2000, Food Hydrocolloids 14: 125-128) followed by gravimetric or cellulose-binding analysis (Fry et al., 1992, Biochem. J. 282: 821-828), or can be assessed colorimetrically by association with iodine under alkaline conditions (Sulova et al., 1995, Analytical Biochemistry 229: 80-85). Incorporation of a functionalized xyloglucan oligomer into a xyloglucan polymer by incubation of the functionalized oligomer with xyloglucan in the presence of xyloglucan endotransglycosylase can be assessed, e.g., by incubating a radiolabeled xyloglucan oligomer with xyloglucan and xyloglucan endotransglycosylase, followed by filter paper-binding and measurement of filter paper radioactivity, or incorporation of a fluorescently or optically functionalized xyloglucan oligomer can be assessed similarly, monitoring fluorescence or colorimetrically analyzing the filter paper.

Xyloglucan oligomer: The term “xyloglucan oligomer” means a short chain xyloglucan oligosaccharide, including single or multiple repeating units of xyloglucan. Most optimally, the xyloglucan oligomer will be 1 to 3 kDa in molecular weight, corresponding to 1 to 3 repeating xyloglucan units.

### DETAILED DESCRIPTION OF THE INVENTION

The present invention relates to methods for modifying a non-cellulosic textile material comprising treating the non-cellulosic textile material with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; and (h) a composition comprising a xyloglucan endotransglycosylase and a xyloglucan oligomer, under conditions leading to a modified non-cellulosic textile material, wherein the modified non-cellulosic textile material possesses a textile improvement compared to the unmodified non-cellulosic textile material.

The present invention also relates to modified non-cellulosic textile materials obtained by such methods.

The present invention also relates to modified non-cellulosic textile materials comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group, and a xyloglucan oligomer; (d) a polymeric xyloglucan, and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

The present invention also relates to a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; and (h) a composition comprising a xyloglucan endotransglycosylase and a xyloglucan oligomer.

In one embodiment, the composition comprises a xyloglucan endotransglycosylase, a polymeric xyloglucan, and a functionalized xyloglucan oligomer comprising a chemical group. In another embodiment, the composition comprises a xyloglucan endotransglycosylase, a polymeric xyloglucan functionalized with a chemical group, and a functionalized xyloglucan oligomer comprising a chemical group. In another embodiment, the composition comprises a xyloglucan endotransglycosylase, a polymeric xyloglucan functionalized with a chemical group, and a xyloglucan oligomer. In another embodiment, the composition comprises a xyloglucan endotransglycosylase, a polymeric xyloglucan, and a xyloglucan oligomer. In another embodiment, the composition comprises a xyloglucan endotransglycosylase and a polymeric xyloglucan functionalized with a chemical group. In another embodiment, the composition comprises a xyloglucan endotransglycosylase and a polymeric xyloglucan. In another embodiment, the composition comprises a xyloglucan endotransglycosylase and a functionalized xyloglucan oligomer comprising a chemical group. In another embodiment, the composition comprises a xyloglucan endotransglycosylase and a xyloglucan oligomer.

The methods of the present invention provide for associating, in a non-covalent but essentially irreversible manner, a non-cellulosic textile material with a polymeric xyloglucan. While polymeric xyloglucan has a strong affinity for cellulose, it was surprising and unexpected that, when treated in the presence of a xyloglucan endotransglycosylase, polymeric xyloglucan has a strong affinity for non-cellulosic textile materials. Non-cellulosic textile materials have the advantage of being strong and durable, however are well known in the art to retain odor, to pill, and to feel unnatural.

The methods of the present invention also provide for delivering functionalization to non-cellulosic textile materials. The polymeric xyloglucan can be functionalized with a wide variety of chemical groups, allowing the associated polymeric xyloglucan to impart improved properties to the non-cellulosic textile materials. The properties include, for example, chemical reactivity, binding or binding-specificity, enhanced water resistance, enhanced UV resistance, enhanced optical or color properties, enhanced or more natural feel and texture, enhanced capacity for blending with natural fibers, reduced pilling, and enhanced wrinkle resistance. It is well known in the art that these properties are of value in textile production, textile care, and textile coatings. The methods of the present invention provide a novel approach for increasing the amount of polymeric xyloglucan functionalized with a chemical group and/or functionalized xyloglucan oligomer comprising a chemical group bound to a non-cellulosic textile material by the activity of xyloglucan endotransglycosylase.

Additionally, the opposing activities of xyloglucanase, endo-β-1-4 glucanase, cellulase, or combinations thereof and xyloglucan endotransglycosylase can be exploited to permit the functionalization to be enzymatically removed and reintroduced as desired. The subsequently unfunctionalized non-cellulosic textile material is refunctionalized by addition of polymeric xyloglucan functionalized with a chemical group or functionalized xyloglucan oligomers and xyloglucan endotransglycosylase.

In one aspect, the functionalization can provide any functionally useful chemical moiety.

The xyloglucan endotransglycosylase is preferably present at about 0.1 nM to about 1 mM, e.g., about 10 nM to about 100 μM or about 0.5 μM to about 5 μM, in the composition.

The polymeric xyloglucan or polymeric xyloglucan functionalized with a chemical group is preferably present at about 10 mg to about 1 g per g of the composition, e.g., about 100 mg to about 950 mg or about 500 mg to about 900 mg per g of the composition.

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 10 mg to about 1 g per g of the composition, e.g., about 100 mg to about 950 mg or about 500 mg to about 900 mg per g of the composition.

When present with polymeric xyloglucan or polymeric xyloglucan functionalized with a chemical group, the xyloglucan oligomer or the functionalized xyloglucan oligomer is preferably present with the polymeric xyloglucan at about 50:1 to about 0.5:1 molar ratio of xyloglucan oligomer or functionalized xyloglucan oligomer to polymeric xyloglucan or polymeric xyloglucan functionalized with a chemical group, e.g., about 10:1 to about 1:1 or about 5:1 to about 1:1 molar ratio of xyloglucan oligomer or functionalized xyloglucan oligomer to polymeric xyloglucan or polymeric xyloglucan functionalized with a chemical group.

The polymeric xyloglucan or polymeric xyloglucan functionalized with a chemical group is preferably present at about 1 mg to about 1 g per g of the non-cellulosic textile material, e.g., about 10 mg to about 500 mg or about 20 mg to about 200 mg per g of the non-cellulosic textile material.

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 1 mg to about 1 g per g of the non-cellulosic textile material, e.g., about 10 mg to about 100 mg or about 20 mg to about 50 mg per g of the non-cellulosic textile material.

When present with polymeric xyloglucan or polymeric xyloglucan functionalized with a chemical group, the xyloglucan oligomer or the functionalized xyloglucan oligomer is preferably present at about 50:1 to about 0.5:1, e.g., about 10:1 to about 1:1 or about 5:1 to about 1:1 molar ratio of xyloglucan oligomer or functionalized xyloglucan oligomer to polymeric xyloglucan or polymeric xyloglucan functionalized with a chemical group.

The xyloglucan endotransglycosylase is preferably present at about 0.1 nM to about 1 mM, e.g., about 10 nM to about 100 μM or about 0.5 μM to about 5 μM during modification of the non-cellulosic textile material.

The concentration of polymeric xyloglucan, polymeric xyloglucan functionalized with a chemical group, xyloglucan oligomer, or functionalized xyloglucan oligomer comprising a chemical group incorporated into the non-cellulosic textile material is about 0.01 g to about 500 mg per g of the non-cellulosic textile material, e.g., about 0.1 g to about 50 mg or about 1 to about 5 mg per g of the non-cellulosic textile material.

### Non-Cellulosic Textile Materials

In the methods of the present invention, the non-cellulosic textile material can be any non-cellulosic textile material.

The non-cellulosic textile material may be a material composed of acetate, acrylic, nylon, olefin, polyester, rayon, spandex, lastex, or mixtures thereof. In one aspect, the non-cellulosic textile material is acetate. In another aspect, the non-cellulosic textile material is acrylic. In another aspect, the non-cellulosic textile material is nylon. In another aspect, the non-cellulosic textile material is olefin. In another aspect, the non-cellulosic textile material is polyester. In another aspect, the non-cellulosic textile material is rayon. In another aspect, the non-cellulosic textile material is spandex. In another aspect, the non-cellulosic textile material is lastex. In another aspect, the non-cellulosic textile material is a blend of two or more non-cellulosic textile materials selected from the group consisting of acetate, acrylic, nylon, olefin, polyester, rayon, spandex, and lastex.

### Textile Improvements

Treatment of a non-cellulosic textile material according to the methods of the present invention imparts a textile improvement to the non-cellulosic textile material.

The textile improvement can be one or more improvements selected from the group consisting of anti-backstaining, anti-pilling, anti-shrinkage, anti-wear, anti-wrinkle, improved color appearance, fabric softness, improved shape retention, improved static control, improved odor control or anti-odor, chemical or flame resistance, anti-UV, water-repellency, anti-microbial, improved association with cellulosic textile in textile blends, and improved tensile strength. In one aspect, the textile improvement is anti-backstaining. In another aspect, the textile improvement is anti-pilling. In another aspect, the textile improvement is anti-shrinkage. In another aspect, the textile improvement is anti-wear. In another aspect, the textile improvement is anti-wrinkle. In another aspect, the textile improvement is improved color appearance. In another aspect, the textile improvement is fabric softness. In another aspect, the textile improvement is improved shape retention. In another aspect, the textile improvement is improved static control. In another aspect, the textile improvement is improved odor control or anti-odor. In another aspect, the textile improvement is chemical or flame resistance. In another aspect, the textile improvement is anti-UV. In another aspect, the textile improvement is water-repellency. In another aspect, the textile improvement is anti-microbial. In another aspect, the textile improvement is improved association with cellulosic textile in textile blends. In another aspect, the textile improvement is improved tensile strength.

The anti-pilling property is measured according to American Society for Testing and Materials (ASTM) method D3511, D3512, D3514, or D4970.

The anti-wear property is measured according to ASTM method D3181, D3884, D3885, or D3886 or American Association of Textile Chemists and Colorists (AATCC) method 93, Abrasion resistance of Fabrics: Accelerotor Method.

The anti-wrinkle property is measured according to AATCC method 66.

The anti-shrinkage property is measured according to AATCC method 135, 150, or 187.

The improved color appearance property is measured according to Hunter LabScan, AATCC method 8, 16M, 17, 61, 117, 172, 181, 188, or 190.

The fabric softness property is measured according to ASTM method D1388 or D5732 or Kawabata Evaluation System for Fabrics (KES-F).

The improved shape retention property is measured according to ASTM method D3786, or AATCC method 135, 150, 187, or 179.

The improved static control property is measured according to AATCC method 135 or 115 or ANSI standard JIS L0217-103.

The improved tensile strength property is measured according to ASTM method D5034, D5035, D5735, D4964, D6614, D6797, or D6775.

The improved anti-odor property is measured according to gas chromatography—mass spectrometry or test olfactory panel assessment.

The improved tearing strength property is measured according to ASTM method D2261, D1424, or D5734.

The improved water resistance is measured according to ASTM method E96, AATCC method 22 or 193.

The improved weather resistance is measured according to AATCC method 169, 186, or 192.

The improved anti-microbial characteristics are measured according to AATCC method 100-1993 or ANSI standard JIS L 1902-1998.

The improved UV-resistance is measured according to ASTM method D6544.

Flame and temperature resistance is measured according to ASTM method D7138, D1230, D4151, D5238, D6413, D6545, D7140, or D7571.

Chemical resistance is measured according to ASTM test method F739, F903, F1001, F1359, F1383, F1407, or F2130.

### Polymeric Xyloglucan

In the methods of the present invention, the polymeric xyloglucan can be any xyloglucan. In one aspect, the polymeric xyloglucan is obtained from natural sources. In another aspect, the polymeric xyloglucan is synthesized from component carbohydrates, UDP- or GDP-carbohydrates, or halogenated carbohydrates by any means used by those skilled in the art. In another aspect, the natural source of polymeric xyloglucan is tamarind seed or tamarind kernel powder, nasturtium, or plants of the genus Tropaeolum particularly Tropaeolum majus. The natural source of polymeric xyloglucan may be seeds of various dicotyledonous plants such as Hymenaea courbaril, Leguminosae-Caesalpinioideae including the genera Cynometreae, Amherstieae, and Sclerolobieae. The natural source of polymeric xyloglucan may also be the seeds of plants of the families Primulales, Annonaceae, Limnanthaceae, Melianthaceae, Pedaliaceae, and Tropaeolaceae or subfamily Thunbergioideae. The natural source of polymeric xyloglucan may also be the seeds of plants of the families Balsaminaceae, Acanthaceae, Linaceae, Ranunculaceae, Sapindaceae, and Sapotaceae or non-endospermic members of family Leguminosae subfamily Faboideae. In another aspect, the natural source of polymeric xyloglucan is the primary cell walls of dicotyledonous plants. In another aspect, the natural source of polymeric xyloglucan may be the primary cell walls of nongraminaceous, monocotyledonous plants.

The natural source polymeric xyloglucan may be extracted by extensive boiling or hot water extraction, or by other methods known to those skilled in the art. In one aspect, the polymeric xyloglucan may be subsequently purified, for example, by precipitation in 80% ethanol. In another aspect, the polymeric xyloglucan is a crude or enriched preparation, for example, tamarind kernel powder. In another aspect, the synthetic xyloglucan may be generated by automated carbohydrate synthesis (Seeberger, 2003, Chem. Commun. 1115-1121), or by means of enzymatic polymerization, for example, using a glycosynthase (Spaduit et al., 2011, J. Am. Chem. Soc. 133:10892-10900).

In one aspect, the average molecular weight of the polymeric xyloglucan ranges from about 2 kDa to about 500 kDa, e.g., about 2 kDa to about 400 kDa, about 3 kDa to about 300 kDa, about 3 kDa to about 200 kDa, about 5 kDa to about 100 kDa, about 5 kDa to about 75 kDa, about 7.5 kDa to about 50 kDa, or about 10 kDa to about 30 kDa. In another aspect, the number of repeating units is about 2 to about 500, e.g., about 2 to about 400, about 3 to about 300, about 3 to about 200, about 5 to about 100, about 7.5 to about 50, or about 10 to about 30. In another aspect, the repeating unit is any combination of G, X, L, F, S, T and J subunits, according to the nomenclature of Fry et al. (Physiologia Plantarum 89: 1-3, 1993). In another aspect, the repeating unit is either fucosylated or non-fucosylated XXXG-type polymeric xyloglucan common to dicotyledons and nongraminaceous monocots. In another aspect, the polymeric xyloglucan is O-acetylated. In another aspect the polymeric xyloglucan is not O-acetylated. In another aspect, side chains of the polymeric xyloglucan may contain terminal fucosyl residues. In another aspect, side chains of the polymeric xyloglucan may contain terminal arabinosyl residues. In another aspect, side chains of the polymeric xyloglucan may contain terminal xylosyl residues.

For purposes of the present invention, references to the term xyloglucan herein refer to polymeric xyloglucan.

### Xyloglucan Oligomer

In the methods of the present invention, the xyloglucan oligomer can be any xyloglucan oligomer. The xyloglucan oligomer may be obtained by degradation or hydrolysis of polymeric xyloglucan from any source. The xyloglucan oligomer may be obtained by enzymatic degradation of polymeric xyloglucan, e.g., by quantitative or partial digestion with a xyloglucanase or endoglucanase (endo-β-1-4-glucanase). The xyloglucan oligomer may be synthesized from component carbohydrates, UDP- or GDP-carbohydrates, or halogenated carbohydrates by any of the manners commonly used by those skilled in the art.

In one aspect, the average molecular weight of the xyloglucan oligomer ranges from 0.5 kDa to about 500 kDa, e.g., about 1 kDa to about 20 kDa, about 1 kDa to about 10 kDa, or about 1 kDa to about 3 kDa. In another aspect, the number of repeating units is about 1 to about 500, e.g., about 1 to about 20, about 1 to about 10, or about 1 to about 3. In the methods of the present invention, the xyloglucan oligomer is optimally as short as possible (i.e., 1 repeating unit, or about 1 kDa in molecular weight) to maximize the solubility and solution molarity per gram of dissolved xyloglucan oligomer, while maintaining substrate specificity for xyloglucan endotransglycosylase activity. In another aspect, the xyloglucan oligomer comprises any combination of G (β-D glucopyranosyl-), X (α-D-xylopyranosyl-(1→6)-β-D-glucopyranosyl-), L (β-D-galactopyranosyl-(1→2)-α-D-xylopyranosyl-(1→6)-β-D-glucopyranosyl-), F (α-L-fuco-pyranosyl-(1→2)-β-D-galactopyranosyl-(1→2)-α-D-xylopyranosyl-(1→6)-β-D-glucopyranosyl-), S (α-L-arabinofurosyl-(1→2)-α-D-xylopyranosyl-(1→6)-β-D-glucopyranosyl-), T (α-L-arabinofurosyl-(1→3)-α-L-arabinofurosyl-(1→2)-α-D-xylopyranosyl-(1-6)-β-D-glucopyranosyl-), and J (α-L-galactopyranosyl-(1→2)-β-D-galactopyranosyl-(1→2)-α-D-xylopyranosyl-(1→6)-β-D-glucopyranosyl-) subunits according to the nomenclature of Fry et al. (Physiologia Plantarum 89: 1-3, 1993). In another aspect, the xyloglucan oligomer is the XXXG heptasaccharide common to dicotyledons and nongraminaceous monocots. In another aspect, the xyloglucan oligomer is O-acetylated. In another aspect, the xyloglucan oligomer is not O-acetylated. In another aspect, side chains of the xyloglucan oligomer may contain terminal fucosyl residues. In another aspect, side chains of the xyloglucan oligomer may contain terminal arabinosyl residues. In another aspect, side chains of the xyloglucan oligomer may contain terminal xylosyl residues.

### Functionalization of Xyloglucan Oligomer and Polymeric Xyloglucan

The xyloglucan oligomer can be functionalized by incorporating any chemical group known to those skilled in the art. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

In one aspect, the chemical group is an aldehyde group.

In another aspect, the chemical group is an amino group. The amino group can be incorporated into polymeric xyloglucan by reductive amination. Alternatively, the amino group can be an aliphatic amine or an aromatic amine (e.g., aniline). The aliphatic amine can be a primary, secondary or tertiary amine. Primary, secondary, and tertiary amines are nitrogens bound to one, two and three carbons, respectively. In one aspect, the primary amine is C₁-C₈, e.g., ethylamine. In another aspect, each carbon in the secondary amine is C₁-C₈, e.g., diethylamine. In another aspect, each carbon in the tertiary amine is C₁-C₈, e.g., triethyamine.

In another aspect, the chemical group is an aromatic group. The aromatic group can be an arene group, an aryl halide group, a phenolic group, a phenylamine group, a diazonium group, or a heterocyclic group.

In another aspect, the chemical group is a carboxyl group. The carboxyl group can be an acyl halide, an amide, a carboxylic acid, an ester, or a thioester.

In another aspect, the chemical group is a halogen group. The halogen group can be fluorine, chlorine, bromine, or iodine.

In another aspect, the chemical group is a hydroxyl group.

In another aspect, the chemical group is a ketone group.

In another aspect, the chemical group is a nitrile group.

In another aspect, the chemical group is a nitro group.

In another aspect, the chemical group is a sulfhydryl group.

In another aspect, the chemical group is a sulfonate group.

The chemical reactive group can itself be the chemical group that imparts a textile improvement to the non-cellulosic textile material.

By incorporation of chemical reactive groups in such a manner, one skilled in the art can further derivatize the incorporated reactive groups with compounds (e.g., macromolecules) that will impart a textile improvement to the non-cellulosic textile material. For example, the incorporated chemical group may react with the compound that imparts the desired property to incorporate that group into the xyloglucan oligomer via a covalent bond. Alternatively, the chemical group may bind to the compound that imparts the desired property in either a reversible or irreversible manner, and incorporate the compound via a non-covalent association. The derivatization can be performed directly on the functionalized xyloglucan oligomer or after the functionalized xyloglucan oligomer is incorporated into polymeric xyloglucan.

Alternatively, the xyloglucan oligomer can be functionalized by incorporating directly a compound that imparts a textile improvement to the non-cellulosic textile material by using a reactive group contained in the compound or a reactive group incorporated into the compound, such as any of the groups described above.

On the other hand, the polymeric xyloglucan can be directly functionalized by incorporating a reactive group as described above. By incorporation of reactive groups directly into polymeric xyloglucan, one of skill in the art can further derivatize the incorporated reactive groups with compounds that will impart a textile improvement to the non-cellulosic textile material. By incorporation of a compound directly into the polymeric xyloglucan, a desired physical or chemical property can also be directly imparted to the non-cellulosic textile material.

In one aspect, the functionalization is performed by reacting the reducing end hydroxyl of the xyloglucan oligomer or the polymeric xyloglucan. In another aspect, a non-reducing hydroxyl group, other than the non-reducing hydroxyl at position 4 of the terminal glucose, can be reacted. In another aspect, the reducing end hydroxyl and a non-reducing hydroxyl, other than the non-reducing hydroxyl at position 4 of the terminal glucose, can be reacted.

The chemical functional group can be added by enzymatic modification of the xyloglucan oligomer or polymeric xyloglucan, or by a non-enzymatic chemical reaction. In one aspect, enzymatic modification is used to add the chemical functional group. In one embodiment of enzymatic modification, the enzymatic functionalization is oxidation to a ketone or carboxylate, e.g., by galactose oxidase. In another embodiment of enzymatic modification, the enzymatic functionalization is oxidation to a ketone or carboxylate by AA9 Family oxidases (formerly glycohydrolase Family 61 enzymes).

In another aspect, the chemical functional group is added by a non-enzymatic chemical reaction. In one embodiment of the non-enzymatic chemical reaction, the reaction is incorporation of a reactive amine group by reductive amination of the reducing end of the carbohydrate as described by Roy et al., 1984, Can. J. Chem. 62: 270-275, or Dalpathado et al., 2005, Anal. Bioanal. Chem. 381: 1130-1137. In another embodiment of non-enzymatic chemical reaction, the reaction is incorporation of a reactive ketone group by oxidation of the reducing end hydroxyl to a ketone, e.g., by copper (II). In another embodiment of non-enzymatic chemical reaction, the reaction is oxidation of non-reducing end hydroxyl groups (e.g., of the non-glycosidic bonded position 6 hydroxyls of glucose or galactose) by (2,2,6,6-tetramethyl-piperidin-1-yl)oxyl (TEMPO), or the oxoammonium salt thereof, to generate an aldehyde or carboxylic acid as described in Bragd et al., 2002, Carbohydrate Polymers 49: 397-406, or Breton et al., 2007, Eur. J. Org. Chem. 10: 1567-1570.

Xyloglucan oligomers or polymeric xyloglucan can be functionalized by a chemical reaction with a compound containing more than one (i.e., bifunctional or multifunctional) chemical functional group comprising at least one chemical functional group that is directly reactive with xyloglucan oligomer or polymeric xyloglucan. In one aspect, the bifunctional chemical group is a hydrocarbon containing a primary amine and a second chemical functional group. The second functional group can be any of the other groups described above. In some aspects, the two functional groups are separated by hydrocarbon chains (linkers) of various lengths as is well known in the art.

Xyloglucan oligomers or polymeric xyloglucan can be functionalized with a compound of interest by step-wise or concerted reaction wherein the xyloglucan oligomer or polymeric xyloglucan is functionalized as described above, and the compound is reactive to the functionalization introduced therein. In one aspect of coupling via a functionalized xyloglucan oligomer, an amino group is first incorporated into the xyloglucan oligomer by reductive amination and a reactive carbonyl is secondarily coupled to the introduced amino group. In another aspect of coupling via an amino-modified xyloglucan oligomer, the second coupling step incorporates a chemical group, compound or macromolecule via coupling an N-hydroxysuccinimidyl (NHS) ester or imidoester to the introduced amino group. In a preferred embodiment, the NHS ester secondarily coupled to the introduced amino group is a component of a mono or bi-functional crosslink reagent. In another aspect of coupling to a functionalized xyloglucan or xyloglucan oligomer, the first reaction step comprises functionalization with a sulfhydryl group, either via reductive amination with an alkylthioamine (NH₂—(CH₂)ₙ—SH) at elevated temperatures in the presence of a reducing agent (Magid et al., 1996, J. Org. Chem. 61: 3849-3862), or via radical coupling (Wang et al., 2009, Arkivoc xiv: 171-180), followed by reaction of a maleimide group to the sulfhydryl. In some aspects, the reactive group in the compound that imparts the desired property is separated from the rest of the compound by a hydrocarbon chain of an appropriate length, as is well described in the art.

Non-limiting examples of compounds of interest that can be used to functionalize polymeric xyloglucan or xyloglucan oligomers, either by direct reaction or via reaction with a xyloglucan-reactive compound, include peptides, polypeptides, proteins, hydrophobic groups, hydrophilic groups, flame retardants, dyes, color modifiers, specific affinity tags, non-specific affinity tags, metals, metal oxides, metal sulfides, fungicides, herbicides, microbicides or microbiostatics, and non-covalent linker molecules.

In one aspect, the compound is a peptide. The peptide can be an antimicrobial peptide, a “self-peptide” designed to reduce allergenicity and immunogenicity, a cyclic peptide, glutathione, or a signaling peptide (such as a tachykinin peptide, vasoactive intestinal peptide, pancreatic polypeptide related peptide, calcitonin peptide, lipopeptide, cyclic lipopeptide, or other peptide).

In another aspect, the compound is a polypeptide. The polypeptide can be a non-catalytically active protein (i.e., structural or binding protein), or a catalytically active protein (i.e., enzyme). The polypeptide can be an enzyme, an antibody, or an abzyme.

In another aspect, the compound is a compound comprising a hydrophobic group. The hydrophobic group can be polyurethane, polytetrafluoroethylene, or polyvinylidene fluoride.

In another aspect, the compound is a compound comprising a hydrophilic group. The hydrophilic group can be methacylate, methacrylamide, or polyacrylate.

In another aspect, the compound is a flame retardant. The flame-retardant can be aluminum hydroxide or magnesium hydroxide. The flame-retardant can also be a compound comprising an organohalogen group or an organophosphorous group.

In another aspect, the compound is a dye or pigment.

In another aspect, the compound is a specific affinity tag. The specific affinity tag can be biotin, avidin, a chelating group, a crown ether, a heme group, a non-reactive substrate analog, an antibody, target antigen, or a lectin.

In another aspect, the compound is a non-specific affinity tag. The non-specific affinity tag can be a polycation group, a polyanion group, a magnetic particle (e.g., magnetite), a hydrophobic group, an aliphatic group, a metal, a metal oxide, a metal sulfide, or a molecular sieve.

In another aspect, the compound is a fungicide. The fungicide can be a compound comprising a dicarboximide group (such as vinclozolin), a phenylpyrrole group (such as fludioxonil), a chlorophenyl group (such as quintozene), a chloronitrobenzene (such as dicloran), a triadiazole group (such as etridiazole), a dithiocarbamate group (such as mancozeb or dimethyldithiocarbamate), or an inorganic molecule (such as copper or sulfur). In another aspect, the fungicide is a bacterium or bacterial spore such as Bacillus or a Bacillus spore.

In another aspect, the compound is a herbicide. The herbicide can be glyphosate, a synthetic plant hormone (such as a compound comprising a 2,4-dichloropenoxyacetic acid group, a 2,4,5-trichlorophenoxyacetic acid group, a 2-methyl-4-chlorophenoxyacetic acid group, a 2-(2-methyl-4-chlorophenoxy)propionic acid group, a 2-(2,4-dichlorophenoxy)propionic acid group, or a (2,4-dichlorophenoxy)butyric acid group), or a compound comprising a triazine group (such as atrazine (2-chloro-4-(ethylamino)-6-isopropylamino)-s-triazine).

In another aspect, the compound is a bactericidal or bacteriostatic compound. The bactericidal or bacteriostatic compound can be a copper or copper alloy (such as brass, bronze, cupronickel, or copper-nickel-zinc alloy), a sulfonamide group (such as sulfamethoxazole, sulfisomidine, sulfacetamide or sulfadiazine), a silver or organo-silver group, TiO₂, ZnO₂, an antimicrobial peptide, or chitosan.

In another aspect, the compound is a non-covalent linker molecule.

In another aspect, the compound is a color modifier. The color modifier can be a dye, fluorescent brightener, color modifier, or mordant (e.g., alum, chrome alum).

By exploiting the introduced amino group, one of skill in the art can specifically incorporate almost any chemical that will impart a textile improvement. Non-limiting examples of chemical groups that will improve the textile and that can be secondarily or tertiarily incorporated are listed herein. To impart water resistance or weather resistance to textile materials, hydrophobic groups (e.g., polyurethane, polytetrafluoroethylene, and polyvinylidene fluoride) may be incorporated. To improve the feel of the textile, hydrophilic groups (e.g., methacylates, methacrylamide, and polyethylene glycol) may be incorporated. To improve the feel of the textile, textile softening groups may be incorporated (e.g., alkyl sulfonates, betaine, and amine oxides). To make a textile more water absorbent, hygroscopic groups may be incorporated (e.g., polyacrylate). To improve the flame resistance or flame retarding properties of a textile, flame retarding groups may be incorporated (e.g., aluminum hydroxide, magnesium hydroxide, organohalogens, and organophosphorous). To improve the odor resistance of textiles, or to generate anti-microbial textiles for use, for example, in hospital garments, and bandages, and dressings, antimicrobial groups, microbiostatic groups, fungicidal groups or herbicidal groups may be incorporated (e.g., silver and organo-silver, TiO₂, antimicrobial peptides, chitosan, copper and organo-copper, and anti-microbial peptides). To improve the appearance of the textile, dye, mordants, color modifiers, and fluorescent brighteners may be incorporated. To impart specific chemical reactivities or affinities to the textile, e.g., toward environmental toxins, analytes, biological, radiological, or chemical compounds, specific affinity tags may be incorporated (e.g., peptides, polypeptides, enzymes, antibodies or other proteins, biotin, avidin, heme, iodine, and indicator dyes) and in this manner, one of skill in the art can generate diagnostic, protective or chemical resistant textiles, e.g., for cleaning wipes or garments. To impart enhanced UV or light-resistance or to impart enhanced optical and conductive properties, metals, metal oxides, or metal sulfides can be incorporated (e.g., TiO₂, AlO₂, or SiO₂). To impart enhanced feel, or enhanced affinity for other chemicals or chemical functional groups, non-covalent linker molecules or non-specific affinity tags (e.g., polycations, polyanions) may be incorporated.

In one aspect, the desired property is chemical reactivity, resistance to, or affinity towards specific chemicals, chemical groups (i.e., protective clothing or equipment, disposable cleaning wipes, functionalized resins/paper for filtering, separation media, and indicator wipes for testing or diagnosis), toxins, metals, salts, ions, polypeptides or desired analytes, including biological, radiological, or chemical groups. In another aspect, the desired property is textile improvement (e.g., anti-backstaining, anti-pilling, anti-shrinkage, anti-wear, anti-wrinkle, improved color appearance, fabric softness, improved shape retention, flame or chemical resistance, improved static control, improved odor control or anti-odor, anti-UV, water-repellency, anti-microbial, improved association with cellulosic textile in textile blends, and/or improved tensile strength). In another aspect, the desired property is water or weather resistance. In another aspect, the desired property is improved optical properties of the non-cellulosic textile. In another aspect, the desired property is improved conductive properties of the non-cellulosic textile. In another aspect, the desired property is enhanced blending to cellulose or enhanced feel or “cottonization” of non-cellulosic textile material.

### Preparation of Modified Non-Cellulosic Textile Materials

In the methods of the present invention, a modified or functionalized non-cellulosic textile material can be prepared from any non-cellulosic textile material. The non-cellulosic textile material can be modified by treating the non-cellulosic textile material with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; or (h) a composition comprising a xyloglucan endotransglycosylase and a xyloglucan oligomer leading to a modified non-cellulosic textile material, wherein the modified textile material possesses a textile improvement compared to the unmodified non-cellulosic textile material.

The methods are exemplified by, but are not limited to, functionalization of polyester with a fluorescent dye, thereby imparting desired optical properties to the textile material. The polyester, for example, can be polyethylene terephthalate. In the methods of the present invention, sheets of polyethylene terephthalate can be incubated in a pH controlled solution, i.e., a buffered solution, (e.g., sodium citrate) from pH 3 to pH 9, e.g., pH 4 to pH 8 or pH 5 to pH 7, at concentrations from about 1 g/L to about 1 kg/L, e.g., about 20 g/L to about 50 g/L or about 30 g/L to about 40 g/L containing xyloglucan endotransglycosylase. The xyloglucan endotransglycosylase can be present at about 0.1 nM to about 1 mM, e.g., about 10 nM to about 100 μM or about 0.5 μM to about 5 μM. In one aspect, the xyloglucan endotransglycosylase is present at a concentration of 3.2 ng to about 32 g of enzyme per g of the textile material, e.g., about 320 μg to about 5.3 mg of enzyme per g of the textile material. The functionalized xyloglucan oligomer can be present with polymeric xyloglucan at about 50:1 to about 0.5:1 molar ratio, e.g., about 10:1 to about 1:1 or about 5:1 to about 1:1 molar ratio. The polymeric xyloglucan can be present at about 1 mg per g of the textile material to about 1 g per g of the textile material, e.g., about 10 mg to about 100 mg or about 20 mg to about 50 mg per g of the textile material. The incubation can last for a sufficiently long period of time as to effect the desired extent of functionalization, e.g., about instantaneously to about 72 hours, e.g., about 15 minutes to about 48 hours, e.g., about 30 minutes to about 24 hours, e.g., about 1 to about 3 hours. In one aspect of the invention, the non-cellulosic material is separated from xyloglucan endotransglycosylase and unbound xyloglucan or functionalized xyloglucan oligomer by washing in, for example, water.

In one aspect of the present invention, the xyloglucan is functionalized prior to functionalization of the non-cellulosic textile materials. The xyloglucan can be incubated in a pH controlled solution, e.g., a buffered solution, with xyloglucan endotransglycosylase and functionalized xyloglucan oligomers yielding functionalized xyloglucan. Functionalized xyloglucan can then be separated from functionalized xyloglucan oligomers by any method known to those skilled in the art, e.g., ethanol precipitation. For example, the reaction mixture can be incubated in 80% (v/v) ethanol for about 1 minute to about 24 hours, e.g., 30 minutes to hours or 12 to 15 hours, centrifuged for an appropriate length of time at an appropriate velocity to pellet the precipitated, functionalized xyloglucan (e.g., 30 minutes at approximately 2000×g), and the supernatants decanted off. The functionalized xyloglucan is then optionally dried. In this aspect, the functionalized xyloglucan is then incubated with xyloglucan endotransglycosylase and the non-cellulosic textile material.

### Sources of Xyloglucan Endotransglycosylases

Any xyloglucan endotransglycosylase that possesses suitable enzyme activity at a pH and temperature appropriate for the methods of the present invention may be used. It is preferable that the xyloglucan endotransglycosylase is active over a broad pH and temperature range. In an embodiment, the xyloglucan endotransglycosylase has a pH optimum in the range of about 3 to about 10. In another embodiment, the xyloglucan endotransglycosylase has a pH optimum in the range of about 4.5 to about 8.5. In another embodiment, the xyloglucan endotransglycosylase has a cold denaturation temperature less than or equal to about 5° C. or a melting temperature of about 100° C. or higher. In another embodiment, the xyloglucan endotransglycosylase has a cold denaturation temperature of less than or equal to 20° C. or a melting temperature greater than or equal to about 75° C.

The source of the xyloglucan endotransglycosylase used is not critical in the present invention. Accordingly, the xyloglucan endotransglycosylase may be obtained from any source such as a plant, microorganism, or animal.

In one embodiment, the xyloglucan endotransglycosylase is obtained from a plant source. Xyloglucan endotransglycosylase can be obtained from cotyledons of the family Fabaceae (synonyms: Leguminosae and Papilionaceae), preferably genus Phaseolus, in particular, Phaseolus aureus. Preferred monocotyledons are non-graminaceous monocotyledons and liliaceous monocotyledons. Xyloglucan endotransglycosylase can also be extracted from moss and liverwort, as described in Fry et al., 1992, Biochem. J. 282: 821-828. For example, the xyloglucan endotransglycosylase may be obtained from cotyledons, i.e., a dicotyledon or a monocotyledon, in particular a dicotyledon selected from the group consisting of azuki beans, canola, cauliflowers, cotton, poplar or hybrid aspen, potatoes, rapes, soy beans, sunflowers, thalecress, tobacco, and tomatoes, or a monocotyledon selected from the group consisting of wheat, rice, corn, and sugar cane. See, for example, WO 2003/033813 and WO 97/23683.

In another embodiment, the xyloglucan endotransglycosylase is obtained from Arabidopsis thaliana (GENESEQP:AOE11231, GENESEQP:AOE93420, GENESEQP: BAL03414, GENESEQP:BAL03622, or GENESEQP:AWK95154); Carica papaya (GENESEQP:AZR75725); Cucumis sativus (GENESEQP:AZV66490); Daucus carota (GENESEQP:AZV66139); Festuca pratensis (GENESEQP:AZR80321); Glycine max (GENESEQP:AWK95154 or GENESEQP:AYF92062); Hordeum vulgare (GENESEQP:AZR85056, GENESEQP:AQY12558, GENESEQP:AQY12559, or GENESEQP:AWK95180); Lycopersicon esculentum (GENESEQP:ATZ45232); Medicago truncatula (GENESEQP:ATZ48025); Oryza sativa (GENESEQP:ATZ42485, GENESEQP:ATZ57524, or GENESEQP:AZR76430); Populus tremula (GENESEQP:AWK95036); Sagittaria pygmaea (GENESEQP:AZV66468); Sorghum bicolor (GENESEQP:BAO79623 or GENESEQP:BAO79007); Vigna angularis (GENESEQP:ATZ61320); or Zea mays (GENESEQP:AWK94916).

In another embodiment, the xyloglucan endotransglycosylase is a xyloglucan endotransglucosylase/hydrolase (XTH) with both hydrolytic and transglycosylating activities. In a preferred embodiment, the ratio of transglycosylation to hydrolytic rates is at least 10⁻² to 10⁷, e.g., 10⁻¹ to 10⁶ or 10 to 1000.

### Production of Xyloglucan Endotransglycosylases

Xyloglucan endotransglycosylase may be extracted from plants. Suitable methods for extracting xyloglucan endotransglycosylase from plants are described Fry et al., 1992, Biochem. J. 282: 821-828; Sulova et al., 1998, Biochem. J. 330: 1475-1480; Sulova et al., 1995, Anal. Biochem. 229: 80-85; WO 95/13384; WO 97/23683; or EP 562 836.

Xyloglucan endotransglycosylase may also be produced by cultivation of a transformed host organism containing the appropriate genetic information from a plant, microorganism, or animal. Transformants can be prepared and cultivated by methods known in the art.

Techniques used to isolate or clone a gene are known in the art and include isolation from genomic DNA or cDNA, or a combination thereof. The cloning of the gene from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

A nucleic acid construct can be constructed to comprise a gene encoding a xyloglucan endotransglycosylase operably linked to one or more control sequences that direct the expression of the coding sequence in a suitable host cell under conditions compatible with the control sequences. The gene may be manipulated in a variety of ways to provide for expression of the xyloglucan endotransglycosylase. Manipulation of the gene prior to its insertion into a vector may be desirable or necessary depending on the expression vector. Techniques for modifying polynucleotides utilizing recombinant DNA methods are well known in the art.

The control sequence may be a promoter, a polynucleotide that is recognized by a host cell for expression of a polynucleotide encoding a xyloglucan endotransglycosylase. The promoter contains transcriptional control sequences that mediate the expression of the xyloglucan endotransglycosylase. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the xyloglucan endotransglycosylase. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the xyloglucan endotransglycosylase. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a xyloglucan endotransglycosylase and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a xyloglucan endotransglycosylase. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a xyloglucan endotransglycosylase and the signal peptide sequence is positioned next to the N-terminus of the propeptide sequence.

The various nucleotide and control sequences may be joined together to produce a recombinant expression vector that may include one or more convenient restriction sites to allow for insertion or substitution of the polynucleotide encoding the xyloglucan endotransglycosylase at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the xyloglucan endotransglycosylase or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMIR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide may be inserted into a host cell to increase production of a xyloglucan endotransglycosylase. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

The host cell may be any cell useful in the recombinant production of a xyloglucan endotransglycosylase, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

The host cells are cultivated in a nutrient medium suitable for production of the xyloglucan endotransglycosylase using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the xyloglucan endotransglycosylase to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the xyloglucan endotransglycosylase is secreted into the nutrient medium, the polypeptide can be recovered directly from the medium. If the xyloglucan endotransglycosylase is not secreted, it can be recovered from cell lysates.

The xyloglucan endotransglycosylase may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The xyloglucan endotransglycosylase may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a whole fermentation broth comprising the polypeptide is recovered. In a preferred aspect, xyloglucan endotransglycosylase yield may be improved by subsequently washing cellular debris in buffer or in buffered detergent solution to extract biomass-associated polypeptide.

The xyloglucan endotransglycosylase may be purified by a variety of procedures known in the art including, but not limited to, chromatography (e.g., ion exchange, affinity, hydrophobic interaction, mixed mode, reverse phase, chromatofocusing, and size exclusion), electrophoretic procedures (e.g., preparative isoelectric focusing), differential solubility (e.g., ammonium sulfate precipitation), PAGE, membrane-filtration or extraction (see, e.g., Protein Purification, Janson and Ryden, editors, VCH Publishers, New York, 1989) to obtain substantially pure polypeptide. In a preferred aspect, xyloglucan endotransglycosylase may be purified by formation of a covalent acyl-enzyme intermediate with xyloglucan, followed by precipitation with microcrystalline cellulose or adsorption to cellulose membranes. Release of the polypeptide is then effected by addition of xyloglucan oligomers to resolve the covalent intermediate (Sulova and Farkas, 1999, Protein Expression and Purification 16(2): 231-235, and Steele and Fry, 1999, Biochemical Journal 340: 207-211).

### Detergent and Fabric Care Compositions and Additives

The present invention also relates to detergent compositions for non-cellulosic textile materials comprising a surfactant and (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The present invention also relates to fabric care compositions for non-cellulosic textile materials comprising a surfactant and (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The present invention also relates to detergent additives for non-cellulosic textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

In another aspect, the detergent compositions or fabric care compositions further comprise one or more additional cleaning composition components. The choice of additional components is within the skill of the artisan and includes conventional ingredients, including the exemplary non-limiting components set forth below.

The choice of components depends on the type of textile to be improved, the type and/or degree of soiling, the temperature at which improvement is to take place, and the formulation of the detergent product. Although components mentioned below are categorized by general header according to a particular functionality, this is not to be construed as a limitation, as a component may comprise additional functionalities as will be appreciated by the skilled artisan.

Examples of detergent compositions are disclosed in WO 97/07202.

Xvloqlucan Endotransqlvcosylase.

In one embodiment of the present invention, the xyloglucan endotransglycosylase can be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The xyloglucan endotransglycosylase of the detergent composition may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, or a nonionic surfactant such as TRITON X-100®, and the composition may be formulated as described in, for example, WO 92/19709 and WO 92/19708.

In one aspect, a xyloglucan endotransglycosylase, a polymeric xyloglucan, and a functionalized xyloglucan oligomer comprising a chemical group are incorporated into the detergent compositions. In another aspect, a xyloglucan endotransglycosylase, a polymeric xyloglucan functionalized with a chemical group, and a functionalized xyloglucan oligomer comprising a chemical group are incorporated into the detergent compositions. In another aspect, a xyloglucan endotransglycosylase, a polymeric xyloglucan functionalized with a chemical group, and a xyloglucan oligomer are incorporated into the detergent compositions. In another aspect, a xyloglucan endotransglycosylase, a polymeric xyloglucan, and a xyloglucan oligomer are incorporated into the detergent compositions. In another aspect, a xyloglucan endotransglycosylase and a polymeric xyloglucan functionalized with a chemical group are incorporated into the detergent compositions. In another aspect, a xyloglucan endotransglycosylase and a polymeric xyloglucan are incorporated into the detergent compositions. In another aspect, a xyloglucan endotransglycosylase and a functionalized xyloglucan oligomer comprising a chemical group are incorporated into the detergent compositions. In another aspect, a xyloglucan endotransglycosylase and a xyloglucan oligomer are incorporated into the detergent compositions.

Surfactants.

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 1% to about 40% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, about 8% to about 12%, about 3% to about 10%, or about 3% to about 5%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN® and TWEEN®, and combinations thereof.

When included therein the detergent will usually contain from about 1% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 1% to about 40% by weight of a zwitterionic surfactant. Non-limiting examples of zwitterionic surfactants include betaine, alkyldimethylbetaine, sulfobetaine, and combinations thereof.

Hydrotropes.

A hydrotrope is a compound that solubilizes hydrophobic compounds in aqueous solutions (or oppositely, polar substances in a non-polar environment). Typically, hydrotropes have both hydrophilic and a hydrophobic character (so-called amphiphilic properties as known from surfactants). However, the molecular structure of hydrotropes generally do not favor spontaneous self-aggregation, see, for example, review by Hodgdon and Kaler, 2007, Current Opinion in Colloid & Interface Science 12: 121-128. Hydrotropes do not display a critical concentration above which self-aggregation occurs as found for surfactants and lipids forming micellar, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Use of hydrotropes in detergent compositions allow, for example, more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders and Co-Builders.

The detergent composition may contain about 0-65% by weight of a detergent builder or co-builder, or a mixture thereof. In a dish washing detergent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-65% by weight of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriamine-pentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA), sulfomethyl-N,N-diacetic acid (SMDA), N-(2-hydroxyethyl)-ethylidenediamine-N,N′,N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, for example, WO 2009/102854 and U.S. Pat. No. 5,977,053.

Bleachinq Systems.

The detergent may contain 0-40%, such as about 5% to about 25%, by weight of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids, and mixtures thereof. Suitable preformed peracids include, but are not limited to, peroxycarboxylic acids and salts, percarbonic acids and salts, perimidic acids and salts, peroxymonosulfuric acids and salts, for example, oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator means herein a compound which reacts with peroxygen bleach like hydrogen peroxide to form a peracid. The peracid thus formed constitutes the activated bleach. Suitable bleach activators useful herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest is disclosed in EP 624154 and particularly preferred in that family is acetyl triethyl citrate (ATC). Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. Exemplary bleaching systems are described in, for example, WO 2007/087258, WO 2007/087244, WO 2007/087259, and WO 2007/087242. Suitable photobleaches may be, for example, sulfonated zinc phthalocyanine

Polymers.

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning, and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO), and polyvinylpyrrolidone-vinylimidazole (PVPVI). Further exemplary polymers include sulfonated polycarboxylates, polyethylene oxide and polypropylene oxide (PEO-PPO), and diquaternium ethoxy sulfate. Other exemplary polymers are disclosed in WO 2006/130575. Salts of the above-mentioned polymers are also contemplated.

Fabric Hueinq Aqents.

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, and mixtures thereof, for example, as described in WO 2005/03274, WO 2005/03275, WO 2005/03276, and EP 1876226. The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, which may be especially preferred when the composition is in the form of a unit dose pouch. Suitable hueing agents are also disclosed in WO 2007/087257 and WO 2007/087243.

Additional Enzymes.

The detergent additive as well as the detergent composition may comprise one or more additional enzymes, such as a protease, lipase, cutinase, amylase, carbohydrase, cellulase, pectinase, mannanase, arabinase, galactanase, xylanase, and oxidase, e.g., a laccase and/or peroxidase.

In general the properties of the one or more enzymes should be compatible with the selected detergent, (i.e., pH optimum, compatibility with other enzymatic and non-enzymatic ingredients, etc.), and should be present in effective amounts.

Cellulases: Suitable cellulases include, in a non-limiting manner, those of bacterial or fungal origin. Chemically modified or protein engineered mutants are included. Suitable cellulases include cellulases from the genera Acremonium, Bacillus, Fusarium, Humicola, Pseudomonas, and Thielavia, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757, and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, and WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307, and PCT/DK98/00299.

Commercially available cellulases include Celluzyme™ and Carezyme™ (Novozymes A/S), Clazinase™ and Puradax HA™ (Genencor International Inc.), and KAC-500(B)™ (Kao Corporation).

Proteases: Suitable proteases include those of bacterial, fungal, plant, viral, or animal origin, e.g., vegetable or microbial origin. Microbial origin is preferred. Chemically modified or protein engineered mutants are included. They may be alkaline proteases, such as serine proteases or metalloproteases. A serine protease may be of the S1 family, such as trypsin, or the S8 family such as subtilisin. A metalloprotease may be a thermolysin from, for example, family M4, or other metalloprotease such as those from M5, M7 or M8 families.

The term “subtilases” refers to a sub-group of serine protease according to Siezen et al., 1991, Protein Eng. 4: 719-737 and Siezen et al., 1997, Protein Science 6: 501-523. Serine proteases are a subgroup of proteases characterized by having a serine in the active site, which forms a covalent adduct with the substrate. The subtilases may be divided into 6 sub-divisions, i.e., the Subtilisin family, the Thermitase family, the Proteinase K family, the Lantibiotic peptidase family, the Kexin family, and the Pyrolysin family.

Examples of subtilases are those derived from Bacillus, such as Bacillus lentus, B. alkalophilus, B. subtilis, B. amyloliquefaciens, Bacillus pumilus, and Bacillus gibsonii described in U.S. Pat. No. 7,262,042 and WO 2009/021867, and subtilisin lentus, subtilisin Novo, subtilisin Carlsberg, subtilisin BPN′, subtilisin 309, subtilisin 147 and subtilisin 168 described in WO 89/06279 and protease PD138 described in WO 93/18140. Other useful proteases may be those described in WO 01/16285, WO 02/26024 and WO 02/16547. Examples of trypsin-like proteases are trypsin (e.g., of porcine or bovine origin) and the Fusarium protease described in WO 89/06270, WO 94/25583, and WO 2005/040372, and the chymotrypsin proteases derived from Cellumonas described in WO 2005/052161 and WO 2005/052146.

A further preferred protease is the alkaline protease from Bacillus lentus DSM 5483, as described in WO 95/23221, and variants thereof which are described in WO 92/21760, WO 95/23221, EP 1921147, and EP 1921148.

Examples of metalloproteases are the neutral metalloprotease described in WO 2007/044993 such as those derived from Bacillus amyloliquefaciens.

Examples of useful proteases are the variants described in: WO 92/19729, WO 96/034946, WO 98/20115, WO 98/20116, WO 99/011768, WO 01/44452, WO 2003/006602, WO 2004/03186, WO 2004/041979, WO 2007/006305, WO 2011/036263, WO 2011/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using BPN′ for numbering. More preferred the subtilase variants may comprise the mutations: S3T, V41, S9R, A15T, K27R, *36D, V68A, N76D, N87S, R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, and T274A (using BPN′ for numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the trade name Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocades N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases: Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 and WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508, and WO 2009/109500.

Preferred commercial lipase products include Lipolase™, Lipex™; Lipolex™ and Lipoclean™ (Novozymes A/S), Lumafast (originally from Genencor), and Lipomax (originally from Gist-Brocades).

Other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/56782), perhydrolases from the CE7 family (WO 2009/67279), and variants of the M. smegmatis perhydrolase, in particular, the S54V variant used in the commercial product Gentle Power Bleach from Huntsman Textile Effects Pte. Ltd. (WO 2010/100028).

Amylases: Suitable amylases which can be used in the present invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylase having SEQ ID NO: 3 in WO 95/10603 or variants having 90% sequence identity to SEQ ID NO: 3 thereof. Preferred variants are described in WO 94/02597, WO 94/18314, and WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Other useful amylases include amylase having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other suitable amylases are hybrid alpha-amylases comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion, or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions: M197T; H156Y+A181T+N190F+A209V+Q264S; or G48A+T49I+G107A+H156Y+A181T+N190F+I201F+A209V+Q264S.

Further useful amylases are amylase having SEQ ID NO: 6 in WO 99/019467 or variants thereof having 90% sequence identity to SEQ ID NO: 6. Preferred variants of SEQ ID NO: 6 are those having a substitution, a deletion, or an insertion in one or more of the following positions: R181, G182, H183, G184, N195, I206, E212, E216 and K269. Particularly preferred amylases are those having deletion in positions R181 and G182, or positions H183 and G184.

Additional useful amylases are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion, or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other useful amylases are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion, or an insertion in one of more of the following positions: 176, 177, 178, 179, 190, 201, 207, 211 and 264.

Further useful amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion, or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions: N128C+K178L+T182G+Y305R+G475K; N128C+K178L+T182G+F202Y+Y305R+D319T+G475K; S125A+N128C+K178L+T182G+Y305R+G475K; or S125A+N128C+T131I+T165I+K178L+T182G+Y305R+G475K wherein the variants are C-terminally truncated and optionally further comprise a substitution at position 243 and/or a deletion at position 180 and/or position 181.

Additional useful amylases are the alpha-amylase having SEQ ID NO: 12 in WO 01/66712 or a variant having at least 90% sequence identity to SEQ ID NO: 12. Preferred amylase variants are those having a substitution, a deletion, or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078, and WO 2013/001087.

Commercially available amylases are Duramyl™, Termamyl™, Fungamyl™ Stainzyme™, Stainzyme Plus™, Natalase™, Liquozyme X and BAN™ (from Novozymes A/S), and Rapidase™, Purastar™/Effectenz™, Powerase and Preferenz S100 (from Genencor International Inc./DuPont).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as described in WO 93/24618, WO 95/10602, and WO 98/15257.

Commercially available peroxidases include Guardzyme™ (Novozymes A/S).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may be, for instance, stabilized by adding a polyol such as propylene glycol, a sugar or sugar alcohol, lactic acid or boric acid according to established methods. Protected enzymes may be prepared according to the method disclosed in EP 238,216.

Adiunct Materials.

Any detergent component known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, carboxymethylcellulose (CMC), and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants.

The detergent compositions of the present invention may also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant Science Series Volume 71, Marcel Dekker, Inc.

Dye Transfer Inhibitinq Aqents.

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, and polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whiteninq Aqent.

The detergent compositions of the present invention may also contain additional components that may tint articles being cleaned, such as fluorescent whitening agents or optical brighteners. Where present the brightener is preferably at a level of about 0.01% to about 0.5%. Any fluorescent whitening agent suitable for use in a laundry detergent composition may be used in the compositions of the present invention. The most commonly used fluorescent whitening agents are those belonging to the classes of diaminostilbene-sulfonic acid derivatives, diarylpyrazoline derivatives, and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate, and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Another fluorescent whitening agent is the commercially available Parawhite KX, supplied by Paramount Minerals and Chemicals, Mumbai, India. Other fluorescent molecules suitable for use in the present invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil Release Polymers.

The detergent compositions of the present invention may also include one or more soil release polymers, which aid the removal of soils from fabrics such as cotton and polyester based fabrics, in particular the removal of hydrophobic soils from polyester based fabrics. The soil release polymers may be, for example, nonionic or anionic terephthalate based polymers, polyvinyl caprolactam and related copolymers, vinyl graft copolymers, and polyester polyamides. See, for example, Chapter 7 in Powdered Detergents, Surfactant Science Series Volume 71, Marcel Dekker, Inc. Another type of soil release polymers are amphiphilic alkoxylated grease cleaning polymers comprising a core structure and a plurality of alkoxylate groups attached to the core structure. The core structure may comprise a polyalkylenimine structure or a polyalkanolamine structure as described in WO 2009/087523. Furthermore, random graft co-polymers are suitable soil release polymers. Suitable graft co-polymers are described in WO 2007/138054, WO 2006/108856 and WO 2006/113314. Other soil release polymers are substituted polysaccharide structures, especially substituted cellulosic structures, such as modified cellulose derivatives described in EP 1867808 or WO 2003/040279. Suitable cellulosic polymers include cellulose, cellulose ethers, cellulose esters, cellulose amides, and mixtures thereof. Other suitable cellulosic polymers include anionically modified cellulose, nonionically modified cellulose, cationically modified cellulose, zwitterionically modified cellulose, and mixtures thereof. Further suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Aqents.

The detergent compositions of the present invention may also include one or more anti-redeposition agents such as carboxymethylcellulose (deletion, or an insertion), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose-based polymers described under soil release polymers above may also function as anti-redeposition agents.

Other Suitable Adiunct Materials.

Other suitable adjunct materials include, but are not limited to, anti-foaming agents, anti-shrink agents, anti-wrinkling agents, bactericides, binders, carriers, dyes, enzyme stabilizers, fabric softeners, fillers, foam regulators, perfumes, pigments, sod suppressors, solvents, and structurants for liquid detergents and/or structure elasticizing agents.

The detergent composition may be in any convenient form, for example, a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments, which can be of any form, shape and material suitable for holding the composition, e.g., without allowing the release of the composition from the pouch prior to water contact. The pouch is made from water soluble film which encloses an inner volume. The inner volume can be divided into compartments of the pouch. Preferred films are polymeric materials, preferably polymers which are formed into a film or sheet. Preferred polymers and copolymers, or derivatives thereof, are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, maltodextrin, poly methacrylates, most preferably polyvinyl alcohol copolymers, and hydroxypropyl methyl cellulose (HPMC). Preferably the level of polymer in the film, for example PVA, is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, US) plus plasticisers like glycerol, ethylene glycerol, propylene glycol, sorbitol, and mixtures thereof. The pouches can comprise a solid laundry cleaning composition or part components and/or a liquid cleaning composition or part components separated by the water soluble film. The compartment for liquid components can be different in composition than compartments containing solids. Ref: (US 2009/0011970).

Detergent ingredients can be separated physically from each other by compartments in water dissolvable pouches or in different layers of tablets. Negative storage interaction between components can thereby be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, or up to about 35% water. Other types of liquids, including without limitation, alkanols, amines, diols, ethers, and polyols may be included in an aqueous liquid or gel. An aqueous liquid or gel detergent may contain from 0-30% organic solvent. A liquid or gel detergent may be non-aqueous.

A granular detergent may be formulated as described in WO 2009/092699, EP 1705241, EP 1382668, WO 2007/001262, U.S. Pat. No. 6,472,364, WO 2004/074419 or WO 2009/102854. Other useful detergent formulations are described in WO 2009/124162, WO 2009/124163, WO 2009/117340, WO 2009/117341, WO 2009/117342, WO 2009/072069, WO 2009/063355, WO 2009/132870, WO 2009/121757, WO 2009/112296, WO 2009/112298, WO 2009/103822, WO 2009/087033, WO 2009/050026, WO 2009/047125, WO 2009/047126, WO 2009/047127, WO 2009/047128, WO 2009/021784, WO 2009/010375, WO 2009/000605, WO 2009/122125, WO 2009/095645, WO 2009/040544, WO 2009/040545, WO 2009/024780, WO 2009/004295, WO 2009/004294, WO 2009/121725, WO 2009/115391, WO 2009/115392, WO 2009/074398, WO 2009/074403, WO 2009/068501, WO 2009/065770, WO 2009/021813, WO 2009/030632, WO 2009/015951, WO 2011/025615, WO 2011/016958, WO 2011/005803, WO 2011/005623, WO 2011/005730, WO 2011/005844, WO 2011/005904, WO 2011/005630, WO 2011/005830, WO 2011/005912, WO 2011/005905, WO 2011/005910, WO 2011/005813, WO 2010/135238, WO 2010/120863, WO 2010/108002, WO 2010/111365, WO 2010/108000, WO 2010/107635, WO 2010/090915, WO 2010/033976, WO 2010/033746, WO 2010/033747, WO 2010/033897, WO 2010/033979, WO 2010/030540, WO 2010/030541, WO 2010/030539, WO 2010/024467, WO 2010/024469, WO 2010/024470, WO 2010/025161, WO 2010/014395, WO 2010/044905, WO 2010/145887, WO 2010/142503, WO 2010/122051, WO 2010/102861, WO 2010/099997, WO 2010/084039, WO 2010/076292, WO 2010/069742, WO 2010/069718, WO 2010/069957, WO 2010/057784, WO 2010/054986, WO 2010/018043, WO 2010/003783, WO 2010/003792, WO 2011/023716, WO 2010/142539, WO 2010/118959, WO 2010/115813, WO 2010/105942, WO 2010/105961, WO 2010/105962, WO 2010/094356, WO 2010/084203, WO 2010/078979, WO 2010/072456, WO 2010/069905, WO 2010/076165, WO 2010/072603, WO 2010/066486, WO 2010/066631, WO 2010/066632, WO 2010/063689, WO 2010/060821, WO 2010/049187, WO 2010/031607, and WO 2010/000636.

### Textile Coating and Finishing Compositions

The present invention also relates to textile coatings for non-cellulosic textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The present invention also relates to textile finishings for non-cellulosic textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The textile coating and finishing compositions can be used for textile, fabric, upholstery, drapery and clothes, during or following textile production, during garment, upholstery or drapery production or following garment, upholstery or drapery production.

In one embodiment, the textile coating and finishing compositions further comprise one or more additional textile coating composition components. The choice of additional components is within the skill of the artisan and includes conventional ingredients, including the exemplary non-limiting components set forth below.

Polymers:

The textile coating compositions of the present invention may also include one or more polymers including, but not limited to, polyvinylacetate, polyvinylchloride, acrylic, non-ionic acrylic, polyurethane, styrene-butadiene, polymeric elastomers, polytetrafluoroethylene, liquid silicone rubber, room temperature vulcanizing.

Other Components:

The textile coating compositions of the present invention may also include, but are not limited to: plasticizers (e.g., phosphate plasticizers, phthalate plasticizers, etc.), catalysts (e.g., platinum, tin), antimony, tin, halogens, formaldehyde, etc.

The present invention is further described by the following examples that should not be construed as limiting the scope of the invention.

### EXAMPLES

### Media and Solutions

COVE agar plates were composed of 342.3 g of sucrose, 252.54 g of CsCl, 59.1 g of acetamide, 520 mg of KCl, 520 mg of MgSO₄.7H₂O, 1.52 g of KH₂PO₄, 0.04 mg of Na₂B4O₇-10H₂O, 0.4 mg of CuSO₄-5H₂O, 1.2 mg of FeSO₄.7H₂O, 0.7 mg of MnSO₄.2H₂O, 0.8 mg of Na₂MoO₄.2H₂O, 10 mg of ZnSO₄.7H₂O, 25 g of Noble agar, and deionized water to 1 liter.

LB medium was composed of 10 g of tryptone, 5 g of yeast extract, 5 g of NaCl, and deionized water to 1 liter.

LB plates were composed of 10 g of tryptone, 5 g of yeast extract, 5 g of NaCl, 15 g of bacteriological agar, and deionized water to 1 liter.

Minimal medium agar plates were composed of 342.3 g of sucrose, 10 g of glucose, 4 g of MgSO₄.7H₂O, 6 g of NaNO₃, 0.52 g of KCl, 1.52 g of KH₂PO₄, 0.04 mg of Na₂B₄O₇.10H₂O, 0.4 mg of CuSO₄.5H₂O, 1.2 mg of FeSO₄.7H₂O, 0.7 mg of MnSO₄.2H₂O, 0.8 mg of Na₂MoO₄.2H₂O, 10 mg of ZnSO₄.7H₂O, 500 mg of citric acid, 4 mg of d-biotin, 20 g of Noble agar, and deionized water to 1 liter.

Synthetic defined medium lacking uridine was composed of 18 mg of adenine hemisulfate, 76 mg of alanine, 76 mg of arginine hydrochloride, 76 mg of asparagine monohydrate, 76 mg of aspartic acid, 76 mg of cysteine hydrochloride monohydrate, 76 mg of glutamic acid monosodium salt, 76 mg of glutamine, 76 mg of glycine, 76 mg of histidine, myo-76 mg of inositol, 76 mg of isoleucine, 380 mg of leucine, 76 mg of lysine monohydrochloride, 76 mg of methionine, 8 mg of p-aminobenzoic acid potassium salt, 76 mg of phenylalanine, 76 mg of proline, 76 mg of serine, 76 mg of threonine, 76 mg of tryptophan, 76 mg of tyrosine disodium salt, 76 mg of valine, and deionized water to 1 liter.

TAE buffer was composed of 4.84 g of Tris base, 1.14 ml of glacial acetic acid, 2 ml of 0.5 M EDTA pH 8.0, and deionized water to 1 liter.

TBE buffer was composed of 10.8 g of Tris base, 5.5 g of boric acid, 4 ml of 0.5 M EDTA pH 8.0, and deionized water to 1 liter.

2XYT plus ampicillin plates were composed of 16 g of tryptone, 10 g of yeast extract, 5 g of sodium chloride, 15 g of Bacto agar, and deionized water to 1 liter. One ml of a 100 mg/ml solution of ampicillin was added after the autoclaved medium was tempered to 55° C.

YP+2% glucose medium was composed of 10 g of yeast extract, 20 g of peptone, 20 g of glucose, and deionized water to 1 liter.

YP+2% maltodextrin medium was composed of 10 g of yeast extract, 20 g of peptone, 20 g of maltodextrin, and deionized water to 1 liter.

### Example 1

### Preparation of Vigna angularis Xyloglucan Endotransglycosylase 16

Vigna angularis xyloglucan endotransglycosylase 16 (VaXET16; SEQ ID NO: 1 [native DNA sequence], SEQ ID NO: 2 [synthetic DNA sequence], and SEQ ID NO: 3 [deduced amino acid sequence]; also referred to as XTH1) was recombinantly produced in Aspergillus oryzae MT3568 according to the protocol described below. Aspergillus oryzae MT3568 is an amrdS (acetamidase) disrupted gene derivative of Aspergillus oryzae JaL355 (WO 2002/40694), in which pyrG auxotrophy was restored by disrupting the A. oryzae arndS gene with the pyrG gene.

The vector pDLHD0012 was constructed to express the VaXET16 gene in multi-copy in Aspergillus oryzae. Plasmid pDLHD0012 was generated by combining two DNA fragments using megaprimer cloning: Fragment 1 containing the VaXET16 ORF and flanking sequences with homology to vector pBM120 (US20090253171), and Fragment 2 consisting of an inverse PCR amplicon of vector pBM120.

Fragment 1 was amplified using primer 613788 (sense) and primer 613983 (antisense) shown below. These primers were designed to contain flanking regions of sequence homology to vector pBM120 (lower case) for ligation-free cloning between the PCR fragments.

Primer 613788 (sense):
(SEQ ID NO: 7)
ttcctcaatcctctatatacacaactggccATGGGCTCGTCCCTCTGGAC
Primer 613983 (antisense):
(SEQ ID NO: 8)
tgtcagtcacctctagttaattaGATGTCCCTATCGCGTGTACACTCG

Fragment 1 was amplified by PCR in a reaction composed of 10 ng of a GENEART® vector pMA containing the VaXET16 synthetic gene (SEQ ID NO: 3 [synthetic DNA sequence]) cloned between the Sac I and Kpn I sites, 0.5 μl of PHUSION® DNA Polymerase (New England Biolabs, Inc., Ipswich, Mass., USA), 20 pmol of primer 613788, 20 pmol of primer 613983, 1 μl of 10 mM dNTPs, 10 μl of 5× PHUSION® HF buffer (New England Biolabs, Inc., Ipswich, Mass., USA), and 35.5 μl of water. The reaction was incubated in an EPPENDORF® MASTERCYCLER® (Eppendorf AG, Hamburg, Germany) programmed for 1 cycle at 98° C. for seconds; and 30 cycles each at 98° C. for 10 seconds, 60° C. for 10 seconds, and 72° C. for 30 seconds. The resulting 0.9 kb PCR product (Fragment 1) was treated with 1 μl of Dpn I (Promega, Fitchburg, Wis., USA) to remove plasmid template DNA. The Dpn I was added directly to the PCR tube, mixed well, and incubated at 37° C. for 60 minutes, and then was column-purified using a MINELUTE® PCR Purification Kit (QIAGEN Inc., Valencia, Calif., USA) according to the manufacturer's instructions.

Fragment 2 was amplified using primers 613786 (sense) and 613787 (antisense) shown below.

613786 (sense):
(SEQ ID NO: 9)
taattaactagaggtgactgacacctggc
613787 (antisense):
(SEQ ID NO: 10)
catggccagttgtgtatatagaggattgagg

Fragment 2 was amplified by PCR in a reaction composed of 10 ng of plasmid pBM120, 0.5 μl of PHUSION® DNA Polymerase, 20 pmol of primer 613786, 20 pmol of primer 613787, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 6.9 kb PCR product (Fragment 2) was treated with 1 μl of Dpn I to remove plasmid template DNA. The Dpn I was added directly to the PCR tube, mixed well, and incubated at 37° C. for 60 minutes, and then column-purified using a MINELUTE® PCR Purification Kit according to the manufacturer's instructions.

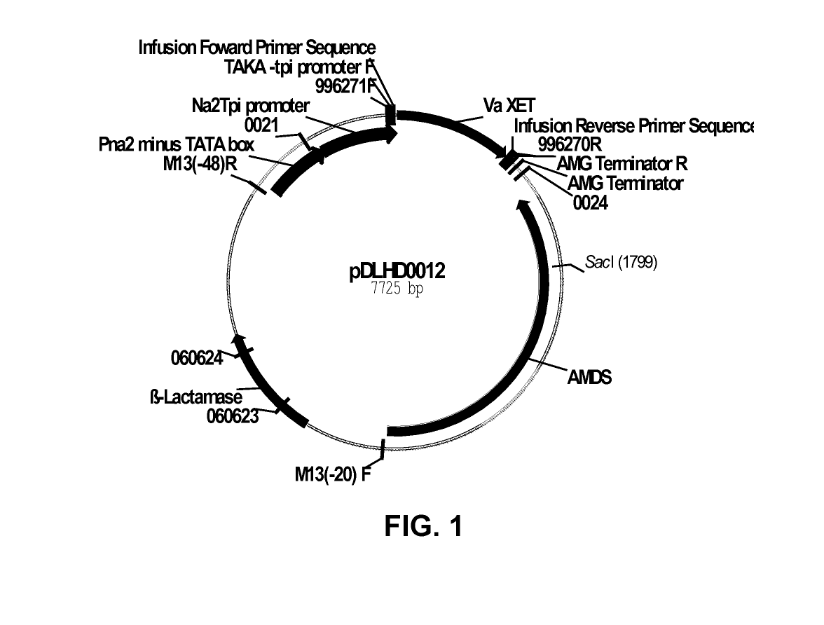

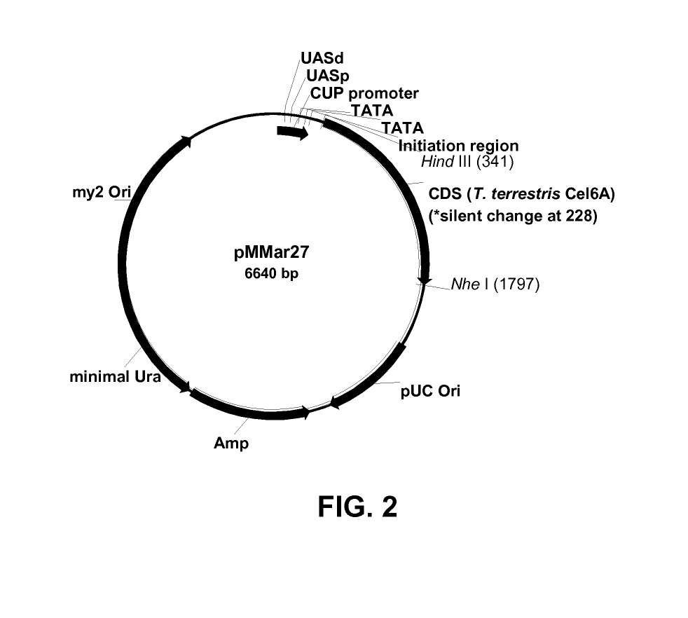

The following procedure was used to combine the two PCR fragments using megaprimer cloning. Fragments 1 and 2 were combined by PCR in a reaction composed of 5 μl of each purified PCR product, 0.5 μl of PHUSION® DNA Polymerase, 1 μl of 10 mM dNTPs, 10 μl of 5× PHUSION® HF buffer, and 28.5 μl of water. The reaction was incubated in an EPPENDORF@ MASTERCYCLER® programmed for 1 cycle at 98° C. for 30 seconds; and 40 cycles each at 98° C. for 10 seconds, 60° C. for 10 seconds, and 72° C. for 4 minutes. Two μl of the resulting PCR product DNA was then transformed into E. coli ONE SHOT® TOP10 electrocompetent cells (Life Technologies, Grand Island, N.Y., USA) according the manufacturer's instructions. Fifty μl of transformed cells were spread onto LB plates supplemented with 100 μg of ampicillin per ml and incubated at 37° C. overnight. Individual transformants were picked into 3 ml of LB medium supplemented with 100 μg of ampicillin per ml and grown overnight at 37° C. with shaking at 250 rpm. The plasmid DNA was purified from the colonies using a QIAPREP® Spin Miniprep Kit (QIAGEN Inc., Valencia, Calif., USA). DNA sequencing using a 3130XL Genetic Analyzer (Applied Biosystems, Foster City, Calif., USA) was used to confirm the presence of each of both fragments in the final plasmid pDLHD0012 (FIG. 1).

Aspergillus oryzae strain MT3568 was transformed with plasmid pDLHD0012 comprising the VaXET16 gene according to the following protocol. Approximately 2-5×10⁷ spores of A. oryzae strain MT3568 were inoculated into 100 ml of YP+2% glucose medium in a 500 ml shake flask and incubated at 28° C. and 110 rpm overnight. Ten ml of the overnight culture were filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCl-20 mM CaCl₂ and transferred to a sterile 125 ml shake flask containing 20 mg of GLUCANEX® 200 G (Novozymes Switzerland AG, Neumatt, Switzerland) per ml and 0.2 mg of chitinase (Sigma-Aldrich, St. Louis, Mo., USA) per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® (Calbiochem, San Diego, Calif., USA) into a sterile 50 ml plastic centrifuge tube to remove mycelial debris. The debris in the MIRACLOTH® was washed thoroughly with 0.7 M KCl-20 mM CaCl₂, and centrifuged at 2500 rpm (537×g) for 10 minutes at 20-23° C. The supernatant was removed and the protoplast pellet was resuspended in 20 ml of 1 M sorbitol-10 mM Tris-HCl (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0012 were added to the bottom of a sterile 2 ml plastic centrifuge tube. Then 100 μl of protoplasts were added to the tube followed by 300 μl of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm COVE agar plates. Transformation plates were incubated at 34° C. until colonies appeared.

Twenty-one transformant colonies were picked to fresh COVE agar plates and cultivated at 34° C. for four days until the transformants sporulated. Fresh spores were transferred to 48-well deep-well plates containing 2 ml of YP+2% maltodextrin, covered with a breathable seal, and grown for 4 days at 34° C. with no shaking. After 4 days growth samples of the culture media were assayed for xyloglucan endotransglycosylase activity using an iodine stain assay and for xyloglucan endotransglycosylase expression by SDS-PAGE.

The iodine stain assay for xyloglucan endotransglycosylase activity was performed according to the following protocol. In a 96-well plate, 5 μl of culture broth were added to a mixture of 5 μl of xyloglucan (Megazyme, Bray, United Kingdom) (5 mg/ml in water), 20 μl of xyloglucan oligomers (Megazyme, Bray, United Kingdom) (5 mg/ml in water), and 10 μl of 400 mM sodium citrate pH 5.5. The reaction mix was incubated at 37° C. for thirty minutes, quenched with 200 μl of a solution containing 14% (w/v) Na₂SO₄, 0.2% KI, 100 mM HCl, and 1% iodine (I₂), incubated in the dark for 30 minutes, and then the absorbance was measured in a plate reader at 620 nm. The assay demonstrated the presence of xyloglucan endotransglycosylase activity from several transformants.

SDS-PAGE was performed using a 8-16% CRITERION® Stain Free SDS-PAGE gel (Bio-Rad Laboratories, Inc., Hercules, Calif., USA), and imaging the gel with a Stain Free Imager (Bio-Rad Laboratories, Inc., Hercules, Calif., USA) using the following settings: 5-minute activation, automatic imaging exposure (intense bands), highlight saturated pixels=ON, color=Coomassie, and band detection, molecular weight analysis and reporting disabled. SDS-PAGE analysis indicated that several transformants expressed a protein of approximately 32 kDa corresponding to VaXET16.

### Example 2

### Construction of Plasmid pMMar27 as a Yeast Expression Plasmid Vector

Plasmid pMMar27 was constructed for expression of the T. terrestris Cel6A cellobiohydrolase II in yeast. The plasmid was generated from a lineage of yeast expression vectors: plasmid pMMar27 was constructed from plasmid pBM175b; plasmid pBM175b was constructed from plasmid pBM143b (WO 2008/008950) and plasmid pJLin201; and plasmid pJLin201 was constructed from pBM143b.

Plasmid pJLin201 is identical to pBM143b except an Xba I site immediately downstream of a Thermomyces lanuginosus lipase variant gene in pBM143b was mutated to a unique Nhe I site. A QUIKCHANGE® II XL Site-Directed Mutagenesis Kit (Stratagene, La Jolla, Calif., USA) was used to change the Xba I sequence (TCTAGA) to a Nhe I sequence (gCTAGc) in pBM143b. Primers employed to mutate the site are shown below.

Primer 999551 (sense):
(SEQ ID NO: 11)
5′-ACATGTCTTTGATAAgCTAGcGGGCCGCATCATGTA-3′
Primer 999552 (antisense):
(SEQ ID NO: 12)
5′-TACATGATGCGGCCCgCTAGcTTATCAAAGACATGT-3′

### Lower Case Represents Mutated Nucleotides.

The amplification reaction was composed of 125 ng of each primer above, 20 ng of pBM143b, 1× QUIKCHANGE® Reaction Buffer (Stratagene, La Jolla, Calif., USA), 3 μl of QUIKSOLUTION® (Stratagene, La Jolla, Calif., USA), 1 μl of dNTP mix, and 1 μl of a 2.5 units/ml Pfu Ultra HF DNA polymerase in a final volume of 50 μl. The reaction was performed using an EPPENDORF® MASTERCYCLER® thermocycler programmed for 1 cycle at 95° C. for 1 minute; 18 cycles each at 95° C. for 50 seconds, 60° C. for 50 seconds, and 68° C. for 6 minutes and 6 seconds; and 1 cycle at 68° C. for 7 minutes. After the PCR, the tube was placed on ice for 2 minutes. One microliter of Dpn I was directly added to the amplification reaction and incubated at 37° C. for 1 hour. A 2 μl volume of the Dpn I digested reaction was used to transform E. coli XL10-GOLD® Ultracompetent Cells (Stratagene, La Jolla, Calif., USA) according to the manufacturer's instructions. E. coli transformants were selected on 2XYT plus ampicillin plates. Plasmid DNA was isolated from several of the transformants using a BIOROBOT® 9600. One plasmid with the desired Nhe I change was confirmed by restriction digestion and sequencing analysis and designated plasmid pJLin201. To eliminate possible PCR errors introduced by site-directed-mutagenesis, plasmid pBM175b was constructed by cloning the Nhe I site containing fragment back into plasmid pBM143b. Briefly, plasmid pJLin201 was digested with Nde I and Mlu I and the resulting fragment was cloned into pBM143b previously digested with the same enzymes using a Rapid Ligation Kit (Roche Diagnostics Corporation, Indianapolis, Ind., USA). Then, 7 μl of the Nde I/Mlu I digested pJLin201 fragment and 1 μl of the digested pBM143b were mixed with 2 μl of 5×DNA dilution buffer (Roche Diagnostics Corporation, Indianapolis, Ind., USA), 10 μl of 2× T4 DNA ligation buffer (Roche Diagnostics Corporation, Indianapolis, Ind., USA), and 1 μl of T4 DNA ligase (Roche Diagnostics Corporation, Indianapolis, Ind., USA) and incubated for 15 minutes at room temperature. Two microliters of the ligation were transformed into XL1-Blue Subcloning-Grade Competent Cells (Stratagene, La Jolla, Calif., USA) cells and spread onto 2XYT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pBM175b.

Plasmid pMMar27 was constructed from pBM175b and an amplified gene of T. terrestris Cel6A cellobiohydrolase II with overhangs designed for insertion into digested pBM175b. Plasmid pBM175b containing the Thermomyces lanuginosus lipase variant gene under control of the CUP I promoter contains unique Hind III and Nhe I sites to remove the lipase gene. Plasmid pBM175 was digested with these restriction enzymes to remove the lipase gene. After digestion, the empty vector was isolated by 1.0% agarose gel electrophoresis using TBE buffer where an approximately 5,215 bp fragment was excised from the gel and extracted using a QIAQUICK® Gel Extraction Kit. The ligation reaction (20 μl) was composed of 1× IN-FUSION® Buffer (BD Biosciences, Palo Alto, Calif., USA), 1×BSA (BD Biosciences, Palo Alto, Calif., USA), 1 μl of IN-FUSION® enzyme (diluted 1:10) (BD Biosciences, Palo Alto, Calif., USA), 99 ng of pBM175b digested with Hind III and Nhe I, and 36 ng of the purified T. terrestris Cel6A cellobiohydrolase II PCR product. The reaction was incubated at room temperature for 30 minutes. A 2 μl volume of the IN-FUSION® reaction was transformed into E. coli XL10-GOLD® Ultracompetent Cells. Transformants were selected on LB plates supplemented with 100 μg of ampicillin per ml. A colony was picked that contained the T. terrestris Cel6A inserted into the pBM175b vector in place of the lipase gene, resulting in pMMar27 (FIG. 2). The plasmid chosen contained a PCR error at position 228 from the start codon, TCT instead of TCC, but resulted in a silent change in the T. terrestris Cel6A cellobiohydrolase II.

### Example 3

### Construction of pEvFz1 Expression Vector

Expression vector pEvFz1 was constructed by modifying pBM120a (U.S. Pat. No. 8,263,824) to comprise the NA2/NA2-tpi promoter, A. niger amyloglucosidase terminator sequence (AMG terminator), and Aspergillus nidulans orotidine-5′-phosphate decarboxylase gene (pyrG) as a selectable marker.

Plasmid pEvFz1 was generated by cloning the A. nidulans pyrG gene from pAILo2 (WO 2004/099228) into pBM120a. Plasmids pBM120a and pAILo2 were digested with Nsi I overnight at 37° C. The resulting 4176 bp linearized pBM120a vector fragment and the 1479 bp pyrG gene insert from pAILo2 were each purified by 0.7% agarose gel electrophoresis using TAE buffer, excised from the gel, and extracted using a QIAQUICK® Gel Extraction Kit.

The 1479 bp pyrG gene insert was ligated to the Nsi I digested pBM120a fragment using a QUICK LIGATION™ Kit (New England Biolabs, Beverly, Mass., USA). The ligation reaction was composed of 1× QUICK LIGATION™ Reaction Buffer (New England Biolabs, Beverly, Mass., USA), 50 ng of Nsi I digested pBM120a vector, 54 ng of the 1479 bp Nsi I digested pyrG gene insert, and 1 μl of T4 DNA ligase in a total volume of 20 μl. The ligation mixture was incubated at 37° C. for 15 minutes followed at 50° C. for 15 minutes and then placed on ice.

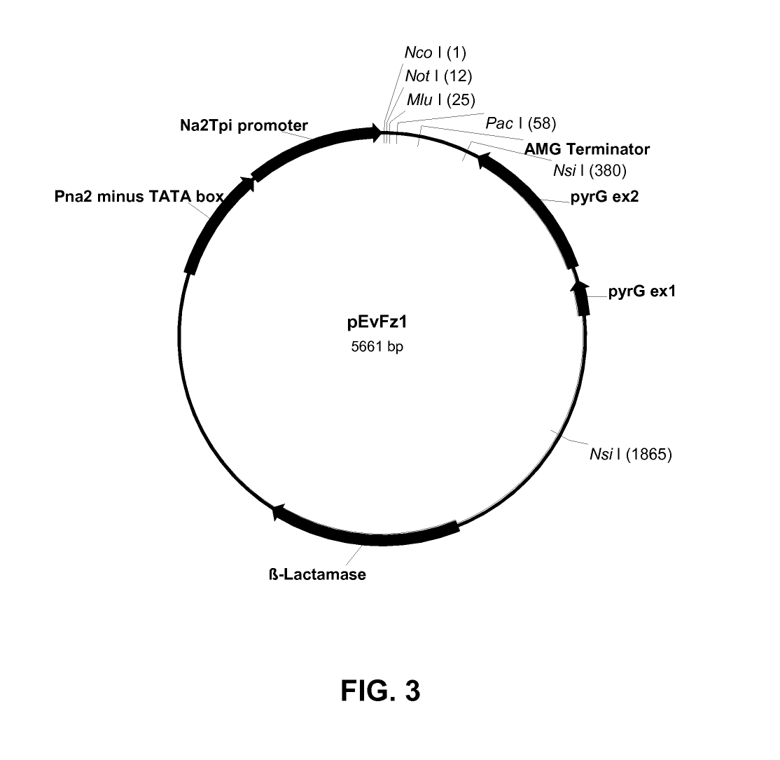

One μl of the ligation mixture was transformed into ONE SHOT® TOP10 chemically competent Escherichia coli cells. Transformants were selected on 2XYT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pEvFz1 (FIG. 3).

### Example 4

### Construction of the Plasmid pDLHD0006 as a Yeast/E. coli A. Oryzae Shuttle Vector

Plasmid pDLHD0006 was constructed as a base vector to enable A. oryzae expression cassette library building using yeast recombinational cloning. Plasmid pDLHD0006 was generated by combining three DNA fragments using yeast recombinational cloning: Fragment 1 containing the E. coli pUC origin of replication, E. coli beta-lactamase (ampR) selectable marker, URA3 yeast selectable marker, and yeast 2 micron origin of replication from pMMar27 (Example 2); Fragment 2 containing the 10 amyR/NA2-tpi promoter (a hybrid of the promoters from the genes encoding Aspergillus niger neutral alpha-amylase and Aspergillus oryzae triose phosphate isomerase and including 10 repeated binding sites for the Aspergillus oryzae amyR transcription factor), Thermomyces lanuginosus lipase open reading frame (ORF), and Aspergillus niger glucoamylase terminator from pJaL1262 (WO 2013/178674); and Fragment 3 containing the Aspergillus nidulans pyrG selection marker from pEvFz1 (Example 3).

| pDLHD0006 | PCR Contents | PCR Template |
|---|---|---|
| Fragment 1 | E. coli ori/AmpR/URA/2 micron (4.1 kb) | pMMar27 |
| Fragment 2 | 10 amyR/NA2-tpi PR/lipase/Tamg | pJaL1262 |
| | (4.5 kb) | |
| Fragment 3 | pyrG gene from pEvFz1 (1.7 kb) | pEvFz1 |

Fragment 1 was amplified using primers 613017 (sense) and 613018 (antisense) shown below. Primer 613017 was designed to contain a flanking region with sequence homology to Fragment 3 (lower case) and primer 613018 was designed to contain a flanking region with sequence homology to Fragment 2 (lower case) to enable yeast recombinational cloning between the three PCR fragments.

Primer 613017 (sense):
(SEQ ID NO: 13)
ttaatcgccttgcagcacaCCGCTTCCTCGCTCACTGACTC
613018 (antisense):
(SEQ ID NO: 14)
acaataaccctgataaatgcGGAACAACACTCAACCCTATCTCGGTC

Fragment 1 was amplified by PCR in a reaction composed of 10 ng of plasmid pMMar27, 0.5 μl of PHUSION® DNA Polymerase (New England Biolabs, Inc., Ipswich, Mass., USA), 20 pmol of primer 613017, 20 pmol of primer 613018, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 1.5 minutes. The resulting 4.1 kb PCR product (Fragment 1) was used directly for yeast recombination with Fragments 2 and 3 below.

Fragment 2 was amplified using primers 613019 (sense) and 613020 (antisense) shown below. Primer 613019 was designed to contain a flanking region of sequence homology to Fragment 1 (lower case) and primer 613020 was designed to contain a flanking region of sequence homology to Fragment 3 (lower case) to enable yeast recombinational cloning between the three PCR fragments.

613019 (sense):
(SEQ ID NO: 15)
agatagggttgagtgttgttccGCATTTATCAGGGTTATTGTCTCATGA
GCGG
613020 (antisense):
(SEQ ID NO: 16)
ttctacacgaaggaaagagGAGGAGAGAGTTGAACCTGGACG

Fragment 2 was amplified by PCR in a reaction composed of 10 ng of plasmid pJaL1262, 0.5 μl of PHUSION® DNA Polymerase, 20 pmol of primer 613019, 20 pmol of primer 613020, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 4.5 kb PCR product (Fragment 2) was used directly for yeast recombination with Fragment 1 above and Fragment 3 below.

Fragment 3 was amplified using primers 613022 (sense) and 613021 (antisense) shown below. Primer 613021 was designed to contain a flanking region of sequence homology to Fragment 2 (lower case) and primer 613022 was designed to contain a flanking region of sequence homology to Fragment 1 (lower case) to enable yeast recombinational cloning between the three PCR fragments.

613022 (sense):
(SEQ ID NO: 17)
aggttcaactctctcctcCTCTTTCCTTCGTGTAGAAGACCAGACAG
613021 (antisense):
(SEQ ID NO: 18)
tcagtgagcgaggaagcggTGTGCTGCAAGGCGATTAAGTTGG

Fragment 3 was amplified by PCR in a reaction composed of 10 ng of plasmid pEvFz1 (Example 3), 0.5 μl of PHUSION® DNA Polymerase, 20 pmol of primer 613021, 20 pmol of primer 613022, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 1.7 kb PCR product (Fragment 3) was used directly for yeast recombination with Fragments 1 and 2 above.

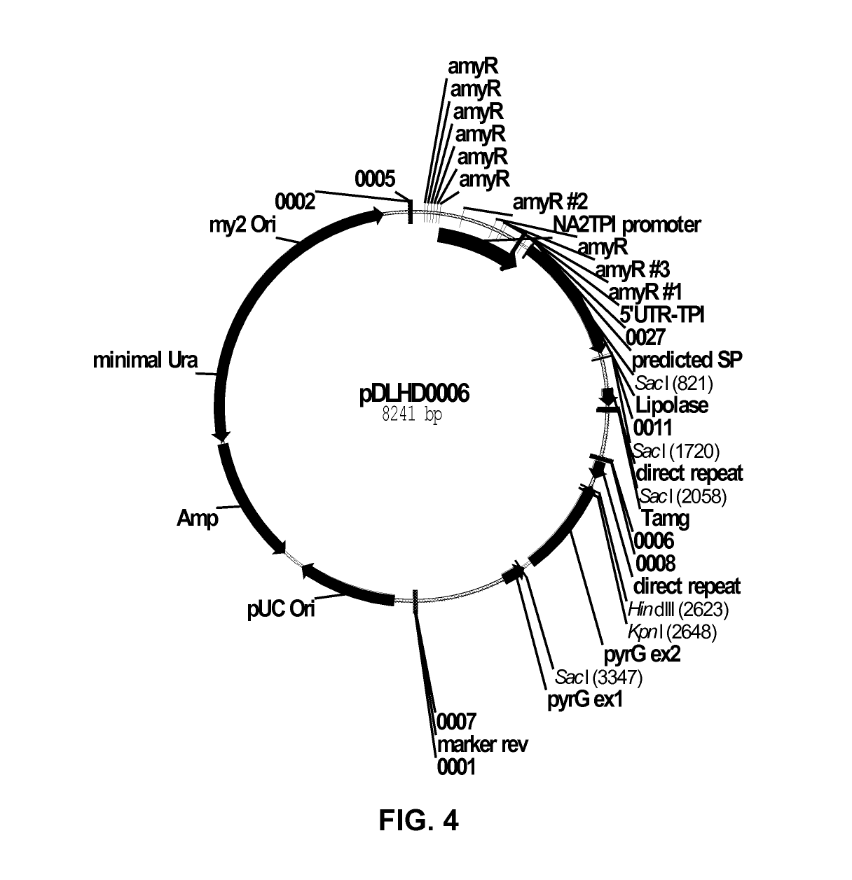

The following procedure was used to combine the three PCR fragments using yeast homology-based recombinational cloning. A 20 μl aliquot of each of the three PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit. A 3 μl aliquot of the plasmid DNA was then transformed into E. coli ONE SHOT® TOP10 electrocompetent cells according the manufacturer's instructions. Fifty μl of transformed cells were spread onto LB plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated pDLHD0006 (FIG. 4).

### Example 5

### Preparation of Arabidopsis thaliana Xyloglucan Endotransglycosylase 14

Arabidopsis thaliana xyloglucan endotransglycosylase (AtXET14; SEQ ID NO: 4 [native DNA sequence], SEQ ID NO: 5 [synthetic DNA sequence], and SEQ ID NO: 6 [deduced amino acid sequence]) was recombinantly produced in Aspergillus oryzae JaL355 (WO 2008/138835).

The vector pDLHD0039 was constructed to express the AtXET14 gene in multi-copy in Aspergillus oryzae. Plasmid pDLHD0039 was generated by combining two DNA fragments using restriction-free cloning: Fragment 1 containing the AtXET14 ORF and flanking sequences with homology to vector pDLHD0006 (Example 4), and Fragment 2 consisting of an inverse PCR amplicon of vector pDLHD0006.

Fragment 1 was amplified using primers AtXET14F (sense) and AtXET14R (antisense) shown below, which were designed to contain flanking regions of sequence homology to vector pDLHD0006 (lower case) for ligation-free cloning between the PCR fragments.

Primer AtXET14F (sense):
(SEQ ID NO: 19)
ttcctcaatcctctatatacacaactggccATGGCCTGTTTCGCAACCA
AACAG
AtXET14R (antisense):
(SEQ ID NO: 20)
agctcgctagagtcgacctaGAGTTTACATTCCTTGGGGAGACCCTG

Fragment 1 was amplified by PCR in a reaction composed of 10 ng of a GENEART® vector pMA containing the AtXET14 synthetic DNA sequence cloned between the Sac I and Kpn I sites, 0.5 μl of PHUSION® DNA Polymerase (New England Biolabs, Inc., Ipswich, Mass., USA), 20 pmol of primer AtXET14F, 20 pmol of primer AtXET14R, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 30 seconds. The resulting 0.9 kb PCR product (Fragment 1) was treated with 1 μl of Dpn I to remove plasmid template DNA. The Dpn I was added directly to the PCR tube, mixed well, and incubated at 37° C. for 60 minutes, and then column-purified using a MINELUTE® PCR Purification Kit.

Fragment 2 was amplified using primers 614604 (sense) and 613247 (antisense) shown below.

614604 (sense):
(SEQ ID NO: 21)
taggtcgactctagcgagctcgagatc
613247 (antisense):
(SEQ ID NO: 22)
catggccagttgtgtatatagaggattgaggaaggaagag

Fragment 2 was amplified by PCR in a reaction composed of 10 ng of plasmid pDLHD0006, 0.5 μl of PHUSION® DNA Polymerase, 20 pmol of primer 614604, 20 pmol of primer 613247, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 7.3 kb PCR product (Fragment 2) was treated with 1 μl of Dpn I to remove plasmid template DNA. Dpn I was added directly to the PCR tube, mixed well, and incubated at 37° C. for 60 minutes, and then column-purified using a MINELUTE® PCR Purification Kit.

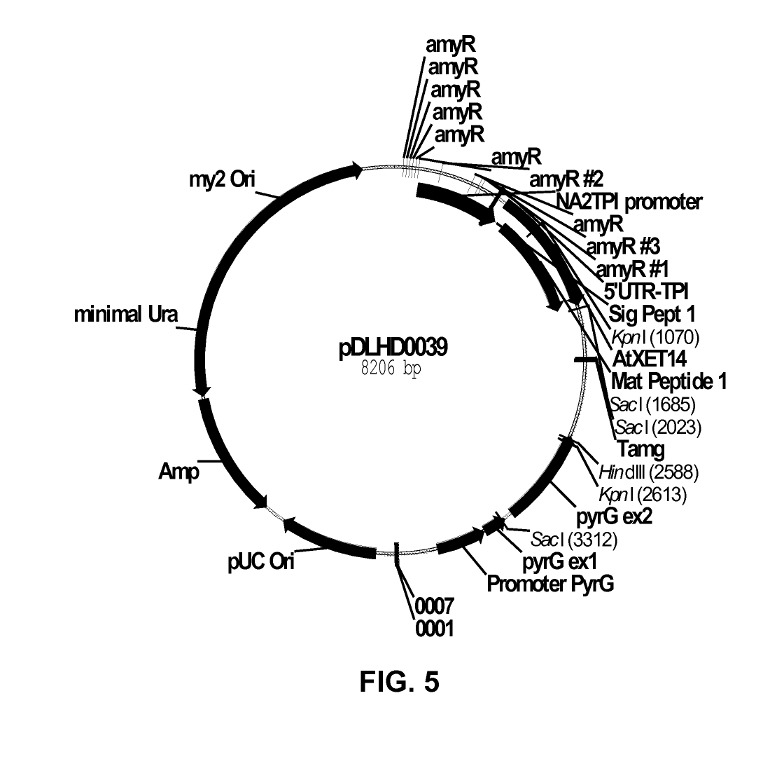

The two PCR fragments were combined using a GENEART® Seamless Cloning and Assembly Kit (Invitrogen, Carlsbad, Calif., USA) according to manufacturer's instructions. Three μl of the resulting reaction product DNA were then transformed into E. coli ONE SHOT® TOP10 electrocompetent cells. Fifty μl of transformed cells were spread onto LB plates supplemented with 100 μg of ampicillin per ml and incubated at 37° C. overnight. Individual transformant colonies were picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit according to the manufacturer's instructions. DNA sequencing with a 3130XL Genetic Analyzer was used to confirm the presence of each of both fragments in the final plasmid pDLHD0039 (FIG. 5).

Aspergillus oryzae strain JaL355 was transformed with plasmid pDLHD0039 comprising the AtXET14 gene according to the following protocol. Approximately 2-5×10⁷ spores of Aspergillus oryzae JaL355 were inoculated into 100 ml of YP+2% glucose+10 mM uridine in a 500 ml shake flask and incubated at 28° C. and 110 rpm overnight. Ten ml of the overnight culture was filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCl-20 mM CaCl₂ and transferred to a sterile 125 ml shake flask containing 20 mg of GLUCANEX® 200 G per ml and 0.2 mg of chitinase per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® into a sterile 50 ml plastic centrifuge tube to remove mycelial debris. The debris in the MIRACLOTH® was washed thoroughly with 0.7 M KCl-20 mM CaCl₂ and centrifuged at 2500 rpm (537×g) for 10 minutes at 20-23° C. The supernatant was removed and the protoplast pellet was resuspended in 20 ml of 1 M sorbitol-10 mM Tris-HCl (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0039 were added to the bottom of a sterile 2 ml plastic centrifuge tube. Then 100 μl of protoplasts were added to the tube followed by 300 μl of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm Minimal medium agar plates. Transformation plates were incubated at 34° C. until colonies appeared.

Thirty-five transformant colonies were picked to fresh Minimal medium agar plates and cultivated at 34° C. for four days until the strains sporulated. Fresh spores were transferred to 48-well deep-well plates containing 2 ml of YP+2% maltodextrin, covered with a breathable seal, and grown for 4 days at 28° C. with no shaking. After 4 days growth the culture medium was assayed for xyloglucan endotransglycosylase activity and for xyloglucan endotransglycosylase expression by SDS-PAGE.

Xyloglucan endotransglycosylase activity was measured using the iodine stain assay described in Example 1. The assay demonstrated the presence of xyloglucan endotransglycosylase activity in several transformants.

SDS-PAGE was performed as described in Example 1. SDS-PAGE analysis indicated that several transformants expressed a protein of approximately 32 kDa corresponding to AtXET14.

### Example 6

### Generation of Fluorescein Isothiocyanate-Labeled Xyloglucan

Fluorescein isothiocyanate-labeled xyloglucan oligomers (FITC-XGOs) were generated by reductive amination of the reducing ends of xyloglucan oligomers (XGOs) according to the procedure described by Zhou et al., 2006, Biocatalysis and Biotransformation 24: 107-120), followed by conjugation of the amino groups of the XGOs to fluorescein isothiocyanate isomer I (Sigma Aldrich, St. Louis, Mo., USA) in 100 mM sodium bicarbonate pH 9.0 for 24 hours at room temperature. Conjugation reaction products were concentrated to dryness in vacuo, dissolved in 0.5 ml of deionized water, and purified by silica gel chromatography, eluting with a gradient from 100:0:0.04 to 70:30:1 acetonitrile:water:acetic acid as mobile phase. Purity and product identity were confirmed by evaporating the buffer, dissolving in D₂O (Sigma Aldrich, St. Louis, Mo., USA), and analysis by ¹H NMR using a Varian 400 MHz MercuryVx (Agilent, Santa Clara, Calif., USA). Dried FITC-XGOs were stored at −20° C. in the dark, and were desiccated during thaw.

Twenty-four ml of 10 mg of tamarind seed xyloglucan (Megazyme, Bray, UK) per ml of deionized water, 217 μl of 7.9 mg of FITC-XGOs per ml of deionized water, 1.2 ml of 400 mM sodium citrate pH 5.5, and 600 μl of 1.4 mg of VaXET16 per ml of 20 mM sodium citrate pH 5.5 were mixed thoroughly and incubated overnight at room temperature. Following overnight incubation, FITC-XG was precipitated by addition of ice cold ethanol to a final volume of 110 ml, mixed thoroughly, and incubated at 4° C. overnight. The precipitated FITC-XG was washed with water and then transferred to Erlenmeyer bulbs. Residual water and ethanol were removed by evaporation using an EZ-2 Elite evaporator (SP Scientific/Genevac, Stone Ridge, N.Y., USA) for 4 hours. Dried samples were dissolved in water, and the volume was adjusted to 48 ml with deionized water to generate a final FITC-XG concentration of 5 mg per ml with an expected average molecular weight of 100 kDa.

### Example 7

### Fluorescence Polarization Assay for Xyloglucan Endotransglycosylation Activity

Xyloglucan endotransglycosylation activity was assessed using the following assay. Reactions of 200 μl containing 1 mg of tamarind seed xyloglucan per ml, 0.01 mg/ml FITC-XGOs prepared as described in Example 6, and 10 μl of appropriately diluted XET were incubated for 10 minutes at 25° C. in 20 mM sodium citrate pH 5.5 in opaque 96-well microtiter plates. Fluorescence polarization was monitored continuously over this time period, using a SPECTRAMAX® M5 microplate reader (Molecular Devices, Sunnyvale, Calif., USA) in top-read orientation with an excitation wavelength of 490 nm, an emission wavelength of 520 nm, a 495 cutoff filter in the excitation path, high precision (100 reads), and medium photomultiplier tube sensitivity. XET-dependent incorporation of fluorescent XGOs into non-fluorescent xyloglucan (XG) results in increasing fluorescence polarization over time. The slope of the linear regions of the polarization time progress curves was used to determine the activity.

### Example 8

### Purification of Vigna angularis Xyloglucan Endotransglycosylase 16

One liter solutions of crude fermentation broth of Vigna angularis were filtered using a 0.22 μm STERICUP® filter (Millipore, Bedford, Mass., USA) and the filtrates were stored at 4° C. Cell debris was resuspended in 1 liter of 0.25% TRITON® X-100 (4-(1,1,3,3-tetramethylbutyl)phenyl-polyethylene glycol; Sigma Aldrich, St. Louis, Mo., USA)-20 mM sodium citrate pH 5.5, incubated at least 30 minutes at room temperature, and then filtered using a 0.22 μm STERICUP® filter. The filtrates containing Vigna angularis xyloglucan endotransglycosylase 16 (VaXET16) were pooled and concentrated to a volume between 500 and 1500 ml using a VIVAFLOW® 200 tangential flow concentrator (Millipore, Bedford, Mass., USA) equipped with a 10 kDa molecular weight cutoff membrane.

The concentrated filtrates were loaded onto a 150 ml Q SEPHAROSE® Big Beads column (GE Healthcare Lifesciences, Piscataway, N.J., USA), pre-equilibrated with 20 mM sodium citrate pH 5.5, and eluted isocratically with the same buffer. The eluent was loaded onto a 75 ml Phenyl SEPHAROSE® HP column (GE Healthcare Lifesciences, Piscataway, N.J., USA) pre-equilibrated in 20% ethylene glycol-20 mM sodium citrate pH 5.5. VaXET16 was eluted using a linear gradient from 20% to 50% of 70% ethylene glycol in 20 mM sodium citrate pH 5.5 over 4 column volumes.

Purified VaXET16 was quantified using a BCA assay (Pierce, Rockford, Ill., USA) in a 96-well plate format with bovine serum albumin (Pierce, Rockford, Ill., USA) as a protein standard at concentrations between 0 and 2 mg/ml and was determined to be 1.40 mg/ml. VaXET16 homogeneity was confirmed by the presence of a single band at approximately 32 kDa using a 8-16% gradient CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

The activity of the purified VaXET16 was determined by measuring the rate of incorporation of fluorescein isothiocyanate-labeled xyloglucan oligomers into tamarind seed xyloglucan (Megazyme, Bray, UK) by fluorescence polarization (as described in Example 7). The apparent activity was 18.5±1.2 P s⁻¹ mg⁻¹.

The purified VaXET16 preparation was tested for background enzyme activities including xylanase, amylase, cellulase, beta-glucosidase, protease, amyloglucosidase, and lipase using standard assays as shown below.

Xylanase activity was assayed using wheat arabinoxylan as substrate at pH 6.0 and 50° C. Xylan hydrolysis was assessed colorimetrically at 405 nm by addition of alkaline solution containing PHBAH. One FXU(S) is defined as the endoxylanase activity using Shearzyme® (Novozymes A/S) as a standard.

Amylase activity was assayed using starch as substrate at pH 2.5 and 37° C. Starch hydrolysis was assessed by measuring the residual starch colorimetrically at 600 nm by addition of iodine solution. One FAU(A) is defined as the acid alpha-amylase activity using acid fungal alpha-amylase (available from Novozymes A/S) as a standard.

Amylase activity was assayed using (4,6-ethylidene(G7)-p-nitrophenyl(G1)-α,D-maltoheptaoside (4,6-ethylidene-G7-pNP) as substrate at pH 7 and 37° C. Hydrolysis of the substrate produces p-nitrophenol, and was assessed colorimetrically at 405 nm. One FAU(F) is defined as fungal alpha-amylase units using Fungamyl® (Novozymes A/S) as a standard.

Cellulase activity was assayed using carboxymethylcellulose (CMC) as substrate at pH 5.0 and 50° C. CMC hydrolysis was assessed colorimetrically at 405 nm by addition of an alkaline solution containing para-hydroxybenzoic acid hydrazide (PHBAH). One CNU(B) is defined as the cellulase activity using NS22084 enzyme (Novozymes A/S) as a standard.

Beta-glucosidase activity was assayed using cellobiose as substrate at pH 5.0 and 50° C. Production of glucose from cellobiose was assessed using a coupled enzyme assay with hexokinase and glucose-6-phosphate dehydrogenase converting glucose to 6-phosphoglucanate following reduction of NAD to NADH at 340 nm. One CBU(B) is defined as the amount of enzyme which releases 2 μmole of glucose per minute using cellobiase as a standard.

The protease assay was performed using an EnzChek® Protease Assay Kit (green fluorescence) (Life Technologies, Inc., Grand Island, N.Y., USA) with casein as substrate at pH 6 or 9 and ambient temperature. One KMTU is defined as a kilo microbial trypsin unit related to the amount of enzyme that produces 1 μmole of p-nitroaniline per minute.

Amyloglucosidase activity was assayed using maltose as substrate at pH 4.3 and 37° C. Conversion of maltose to glucose was assessed using a coupled enzyme assay with hexokinase and glucose-6-phosphate dehydrogenase converting glucose to 6-phosphoglucanate following reduction of NAD to NADH at 340 nm. One AGU is defined as amyloglucosidase units using AMG® (Novozymes A/S) as a standard.

The 4-methylumbelliferyl beta-D-lactoside (MUL) assay was performed at pH 7 and ambient temperature and measured fluorometrically at 360 nm excitation and 465 nm emission.

Lipase activity was assayed using 4-nitropenyl butyrate (pNP-butyrate) as substrate at pH 7.5 and ambient temperature. pNP-butyrate hydrolysis was assessed colorimetrically following p-nitrophenol release at 405 nm. One LU is defined as the amount of enzyme which releases 1 μmole of titratable butyric acid using LIPOLASE® (Novozymes A/S) as a standard.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethylidene- | 4-fold | FAU(F) | ND |
| | G7-pNp | | | |
| Cellulase CNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 740 |
| (EnzCheck) | | | | |
| Protease, pH 9 | Casein | none | KMTU | 332 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.02 |

### Example 9

### Purification of Arabidopsis thaliana Xyloglucan Endotransglycosylase 14

The purification and quantification of the Arabidopsis thaliana xyloglucan endotransglycosylase 14 (AtXET14) was performed as described for VaXET16 in Example 8, except that elution from the Phenyl SEPHAROSE® HP column was performed using a linear gradient from 40% to 90% of 70% ethylene glycol in 20 mM sodium citrate pH 5.5 over 4 column volumes.

AtXET14 homogeneity was confirmed by the presence of a single band at approximately 32 kDa using a 8-16% CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

Purified AtXET14 was quantified using a BCA assay in a 96-well plate format with bovine serum albumin as a protein standard at concentrations between 0 and 2 mg/ml and was determined to be 1.49 mg/ml.

The activity of the purified AtXET14 was determined as described in Example 7. The apparent activity was 34.7±0.9 P s⁻¹ mg⁻¹.

The purified AtXET14 preparation was tested for background activities including xylanase, amylase, cellulase, beta-glucosidase, protease, amyloglucosidase, and lipase using standard assays as shown below. The standard assays are described in Example 8.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethylidene- | 4-fold | FAU(F) | ND |
| | G7-pNp | | | |
| Cellulase CNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 82 |
| (EnzCheck) | | | | |
| Protease, pH 9 | Casein | none | KMTU | 53 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.24 |

### Example 10

### Enhancement of Binding of Fluorescein Isothiocyanate-Labeled Xyloglucan

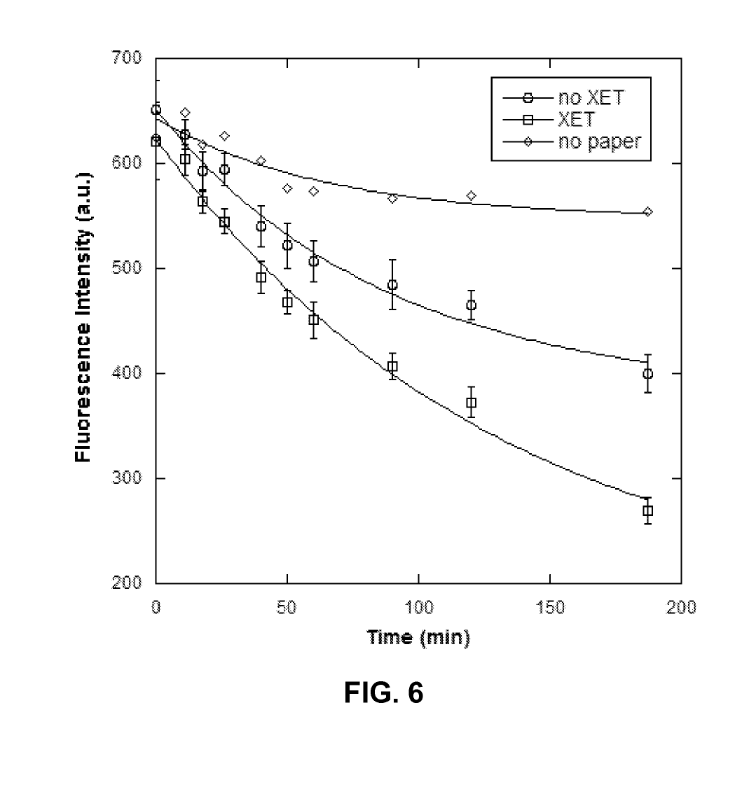

Binding to cellulose by Vigna angularis xyloglucan endotransglycosylase 16 Binding of fluorescein isothiocyanate-labeled xyloglucan (FITC-XG) to filter paper was assessed according to the following protocol. Circular cuttings of Whatman #1 filter paper were generated using a 0.5 inch diameter circular paper punch. Six replicate cuttings of the filter paper were each covered with a 2 ml volume of 317 nM FITC-XG as assessed by absorbance at 488 nm with or without 2.2 μM VaXET16 in Costar #3513, 12-well cell culture cluster plates (Corning, Tewksbury, Mass., USA). Solutions were mixed by pipetting up and down over the surface of the filter paper, and then the plates were incubated at room temperature with gentle shaking on a rocking platform (VWR, Radnor, Pa., USA) at 3 rpm for up to 3 hours. Negative control incubations on each plate contained no paper. At the times indicated in FIG. 6, 1 ml aliquots of the solution phase were removed, the fluorescence intensities were measured, and then the aliquots were returned to the incubation wells. Fluorescence intensities were measured in 1 ml disposable cuvettes using a SPECTRAMAX® M5 spectrophotometer (Molecular Devices, Sunnyvale, Calif., USA) with the following optical parameters: λₑₓ=490 nm, λₑₘ=520 nm, using a 495 nm cut-off filter in the excitation path, photomultiplier tube sensitivity was high, and maximum number of sample reads (100) for precision. Intensities were plotted as the mean and standard deviation of the replicate samples.

FIG. 6 shows the fluorescence intensity of the solution phase of FITC-XG incubated with filter paper, incubated with filter paper in the presence of VaXET16, or incubated with no filter paper. The fluorescence intensity of the solution decreased with time for all samples. The control incubation that contained no filter paper showed a small loss of fluorescence (<15%) likely due to adsorption of the FITC-XG to the culture plate walls and/or due to photobleaching of the fluorescein. The incubation of FITC-XG and filter paper without VaXET16 showed a 38% loss of intensity in 3 hours. The incubation of FITC-XG and filter paper with VaXET16 showed a 55% loss of intensity over the same 3 hour incubation time. Data were fit with a single exponential. The intensities at equilibrium determined from the data fit were 547±18.9, 380±25.5, and 170±53 for FITC-XG incubated with no filter paper, with filter paper, and with filter paper and VaXET16, respectively.

### Example 11

### Enhancement of Binding of Fluorescein Isothiocyanate-Labeled Xyloglucan to Polyethylene Terephthalate by Vigna angularis Xyloglucan Endotransglycosylase 16 and Arabidopsis thaliana Xyloglucan Endotransglycosylase 14

Binding of fluorescein isothiocyanate-labeled xyloglucan (FITC-XG) to polyester (polyethylene terephthalate, or PET) was assessed as described in Example 10 with the following exceptions. A 1 mg per ml solution of Arabidopsis thaliana XET14 (AtXET14) and Vigna angularis XET16 (VaXET16) was used as the xyloglucan endotransglycosylase enzyme solution (XET solution). Bovine serum albumin (BSA) was run as a control at 1 mg per ml. Triplicate binding reactions were performed where 10 mg of PET interfacing fabric or no PET were incubated with 0.125 mg of FITC-XG per ml in the presence and absence of 1.5 μM XET in 50 mM sodium citrate pH 5.5. The reaction mixture was incubated for 2 days at room temperature. After 2 days, the reaction was centrifuged at 3000 rpm for 5 minutes using a LEGEND™ RT Plus centrifuge (Thermo Scientific, Waltham, Mass., USA). Totally, 100 μl of the solution phase of each reaction were transferred to a Costar 9017 flat bottomed microtiter plate (Corning, Tewksbury, Mass., USA), and the fluorescence was measured as described in Example 10.

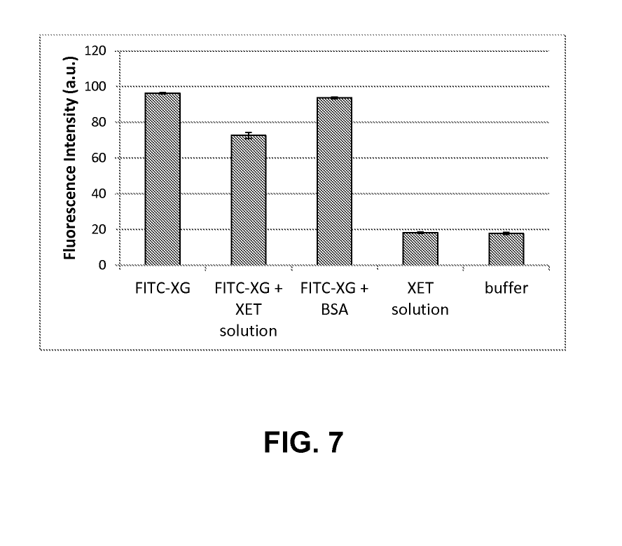

FIG. 7 shows the residual fluorescence intensity in solution following incubation. The fluorescence intensity of the FITC-XG in solution was 25% lower when FITC-XG was incubated with PET in the presence of XET solution than without the XET solution or with BSA. A control incubation containing no PET fabric showed only a marginal decrease in intensity when incubated with the XET solution.

### Example 12

### Enhancement of Binding of Fluorescein Isothiocyanate-Labeled Xyloglucan to Polyethylene Terephthalate by Vigna angularis Xyloglucan Endotransglycosylase 16 Observed by Laser Scanning Confocal Microscopy

Binding reactions of fluorescein isothiocyanate-labeled xyloglucan (FITC-XG) to PET were performed as described in Example 11 with the following exceptions. PET interfacing fabric was cut into small discs using a standard paper punch. Discs of PET were incubated in 0.5 ml of 50 mM sodium citrate pH 5.5, with or without 62.5 μg of FITC-XG with or without 1.5 μM VaXET16. Discs were washed extensively with deionized water and stored in 1 ml of deionized water for 1 week prior to microscopy.

Laser scanning confocal microscopy was performed in the following manner. Small sections of each PET disc were cut with a razor blade and applied to a FisherFinest Premium 3×1″×1 mm microscope slide (Fisher Scientific, Inc., Pittsburgh, Pa., USA) using tweezers.

Three samples, PET incubated in sodium citrate, PET incubated with FITC-XG without VaXET16, and PET incubated with FITC-XG were applied to the same slide. Approximately 20 μl of deionized water were applied to cover the samples and a Fisherbrand 22×22−1.5 microscope coverslip (Fisher Scientific, Inc., Pittsburgh, Pa., USA) was laid over each sample before sealing the coverslip to the slide with nail polish.

Fluorescence arising from the FITC-XG associated with PET was imaged using an Olympus FV1000 laser scanning confocal microscope with a 10× air gap objective lens or a 40× oil immersion lens. Excitation was performed using the 488 nm line of the argon ion laser, and emission intensity was detected by integrating intensity from 500 to 520 nm incident on the photomultiplier tube detector through an emission monochromator. Post scan image analysis was performed using FIJI and MATLAB® (The Mathworks, Natick, Mass., USA).

Laser intensity and photomultiplier tube voltage were initially set to avoid detector saturation and then were maintained to allow comparison between the 3 samples. Confocal images of the 3 samples in the same z-axis plane of the interior of the PET fabric in which the entire field of view is filled by the fabric were obtained and analyzed using both FIJI and MATLAB®.

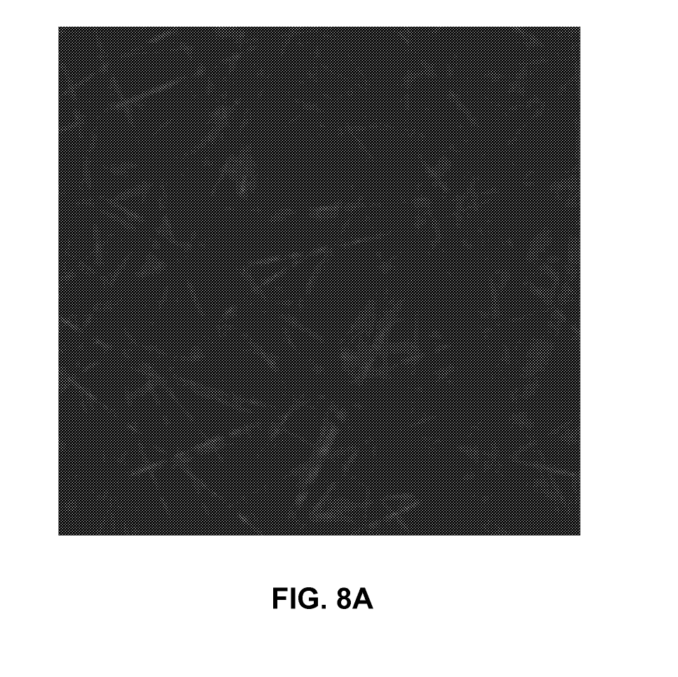

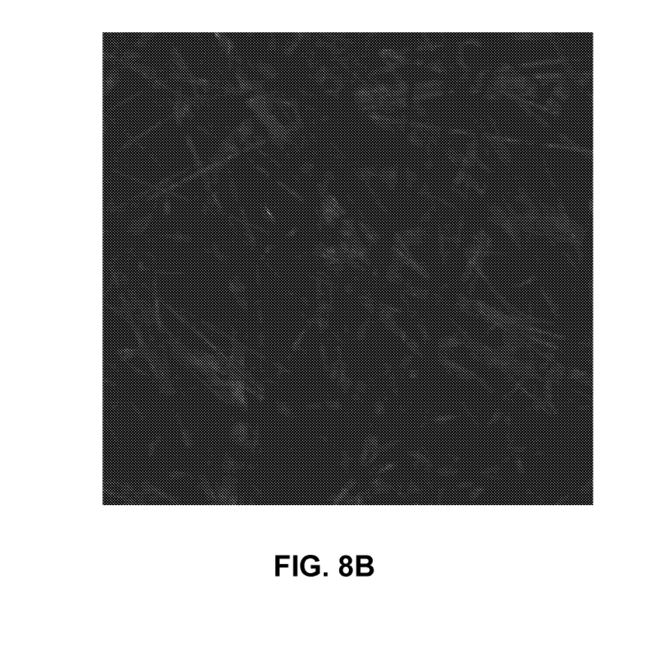

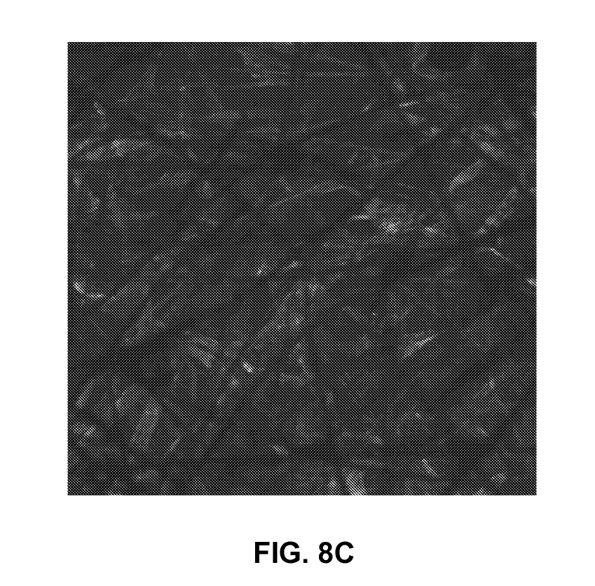

FIG. 8A shows the confocal microscopy image of PET incubated in the absence of FITC-XG at 10× magnification. FIG. 8B shows the confocal microscopy image of PET incubated with FITC-XG at 10× magnification. FIG. 8C shows the confocal microscopy image of PET incubated in the presence of both FITC-XG and the VaXET16 at 10× magnification.

In a comparison of the 3 images, generated with equivalent excitation and emission settings to permit quantitative assessment, the PET incubated with both FITC-XG and VaXET16 clearly had the most intense FITC-XG fluorescence. Quantitative analyses were performed on the images, and the average intensities for non-zero intensity pixels were determined. Excluding background and black space, the average pixel intensities were 14.5, 14.1, and 17.4 a.u. per pixel for the PET incubated with no FITC-XG, with FITC-XG, and with FITC-XG and VaXET16, respectively. These results indicate that in the absence of VaXET16, no FITC-XG was bound within the PET fabric.

Images of the edges of the 3 PET samples showed intense fluorescent foci for the 2 samples incubated with FITC-XG, and no foci for the PET incubated without FITC-XG. These data indicate that xyloglucan can bind to the outside edges of polyester, but that xyloglucan endotransglycosylase activity was required for binding of xyloglucan within the polyester sheet.

### Example 13

### Enhancement of Binding of Fluorescein Isothiocyanate-Labeled Xyloglucan of Various Average Molecular Weights to Cellulose and Polyethylene Terephthalate by Vigna angularis Xyloglucan Endotransglycosylase 16

Fluorescein isothiocyanate-labeled xyloglucan (FITC-XG) of various molecular weights was assessed for binding to cellulose and polyester (polyethylene terephthalate, PET) and binding-enhancement by VaXET16. Endotransglycosylation activity between a low molecular weight xyloglucan oligosaccharide (XGO) and a high molecular weight xyloglucan polymer (XG) is expected to reduce the average molecular weight of the xyloglucan to an extent dependent on the initial ratio of the concentrations of the two components. A series of VaXET16-dependent transglycosylation reactions were performed where the molar ratio of FITC-XG to FITC-XGO was varied; 1:0, 1:1, 1:2, 1:4, 1:8 and 1:16. A fixed volume of 25 μl of 1.5 mg of VaXET16 per ml of 40 mM sodium citrate pH 5.5 was incubated overnight at 25° C. with a fixed volume, 1 ml, of 5 mg of FITC-XG per ml of 50 mM sodium citrate pH 5.5 and varying volumes of 7.9 mg of FITC-XGO per ml of 50 mM sodium citrate pH 5.5 from 0-147 μl to generate FITC-XG with expected molecular weights between 100 and 3.125 kDa. Following the transglycosylation reactions, VaXET16 was inactivated by incubation at 90° C. for 30 minutes.

Various molecular weights of FITC-XG were then assessed for binding to cellulose or polyester as described in Example 11, with the following exceptions. Binding reactions of 200 μl were performed. Roughly 25 μM of each FITC-XG preparation based on expected molecular weight was incubated in 20 mM sodium citrate pH 5.5, with or without 1 μM VaXET16, and either 5 discs of PET (approximately 5 mg) or 1 disc of Whatman #1 filter paper (approximately 3 mg) for 48 hours at 25° C. with shaking at 150 rpm in an INNOVA® 40 shaker incubator (New Brunswick Scientific, Enfield, Conn., USA). Cellulose-bound and polyester-bound fractions in each binding reaction were determined as described in Example 11.

To confirm that the average molecular weight of the FITC-XG polymer had decreased as FITC-labeled oligomers were incorporated, both fluorescence intensity and fluorescence polarization were measured.

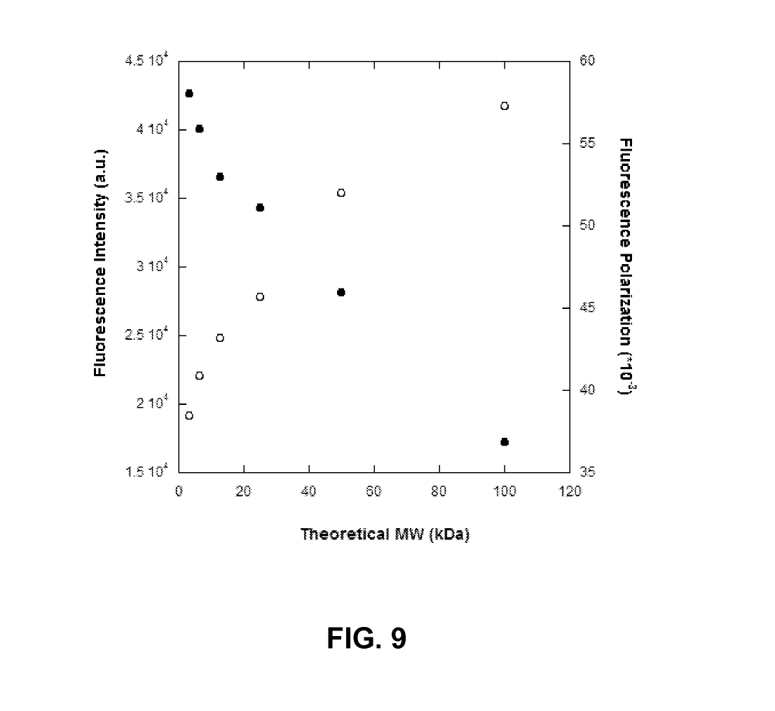

FIG. 9 shows the fluorescence intensity and the fluorescence polarization of the polymers at the indicated expected molecular weight averages. For FITC-XG generated with a higher ratio of XGO to XG and hence smaller expected average molecular weight, the polarization was lower and the fluorescence intensity was higher, whereas for FITC-XG generated with a lower ratio of XGO to XG and hence larger expected molecular weight, polarization was higher and intensity was lower. These results indicate that for higher ratios of XGO to XG, more FITC-XGO was incorporated to generate a larger number of smaller FITC-XG molecules.

To further confirm that the FITC-XGO was incorporated into the FITC-XG, the various expected average molecular weights were analyzed by size exclusion chromatography. Volumes of 1 to 2 ml of each FITC-XG preparation were injected onto a HiLoad 26/60 preparative SUPERDEX® 200 size exclusion column on an Akta Explorer FPLC (GE Healthcare Lifesciences, Piscataway, N.J., USA). Four ml fractions were collected and assayed by fluorescence intensity as described in Example 11 using an excitation wavelength of 490 nm, an emission wavelength of 520 nm, and a 495 nm cutoff filter in the emission path.

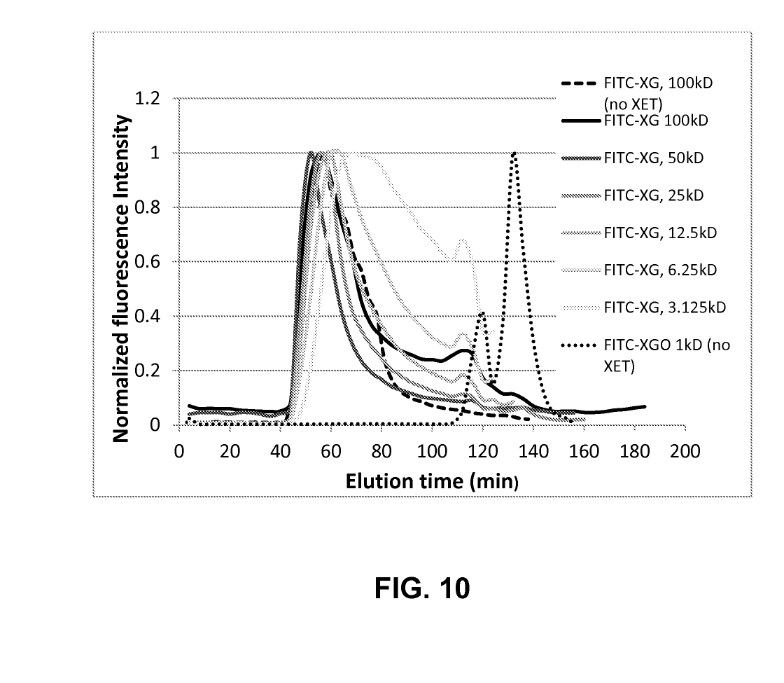

FIG. 10 shows the chromatogram of the various expected molecular weight average FITC-XGs. As the expected molecular weight decreased below 25 kDa, the retention time increased substantially, indicating that the actual molecular weight had decreased. Incubation of the starting material, FITC-XG with VaXET16 but no FITC-XGO also caused a substantial fraction of the material to decrease in size. This was due to an increase in the width of the molecular weight distribution caused by transglycosylation activity.

Fractions were combined into 4 pools based on elution time from the size exclusion chromatography column: pool 1 was collected between 40 and 60 minutes; pool 2 was collected between 61 and 80 minutes; pool 3 was collected between 81 and 100 minutes; and pool 4 was collected between 101 and 120 minutes.

As a final control to confirm the reduction in average molecular weight of the FITC-XG from the initial VaXET16-dependent reactions, the pooled size exclusion chromatography fractions were hydrolyzed and the apparent molar ratio of reducing ends to fluorescent molecule was determined. Each of the 4 combined size exclusion fractions, or no FITC-XG, was incubated with 2.5 μl of CELLIC® CTec3 (Novozymes A/S, Bagsvaerd, Denmark) in 40 mM sodium citrate pH 5.5 at 50° C. for 24 hours in 200 μl hydrolysis reactions. Reactions were performed in 500 μl Nunc U96 PP polypropylene 96-well plates, sealed using an ALPS 3000 plate sealer, and incubated in an Isotemp plus incubator (Fisher Scientific, Waltham, Mass., USA).

The carbohydrate in each fraction was determined using a reducing sugar assay based on the p-hydroxybenzoic acid hydrazide (PHBAH) assay (Lever, 1972, Analytical Biochemistry 47(1): 273-279). Briefly, a 100 μl aliquot of each diluted sample was incubated with 50 μl of 1.5% (w/v) PHBAH in 2% sodium hydroxide at 95° C. for 10 minutes. The reactions were cooled to room temperature, diluted appropriately with deionized water, and the absorbance of 100 μl aliquots was measured at 410 nm using a SPECTRAMAX® 340PC microplate reader (Molecular Devices, Sunnyvale, Calif., USA) in Costar 9017 flat bottomed, microtiter plates. The concentration value in glucose equivalents was determined by comparison to a standard curve of known glucose concentrations treated similarly.

The fluorophore concentration in each pool was estimated by fluorescence intensity in comparison to a standard curve of FITC-XGO.

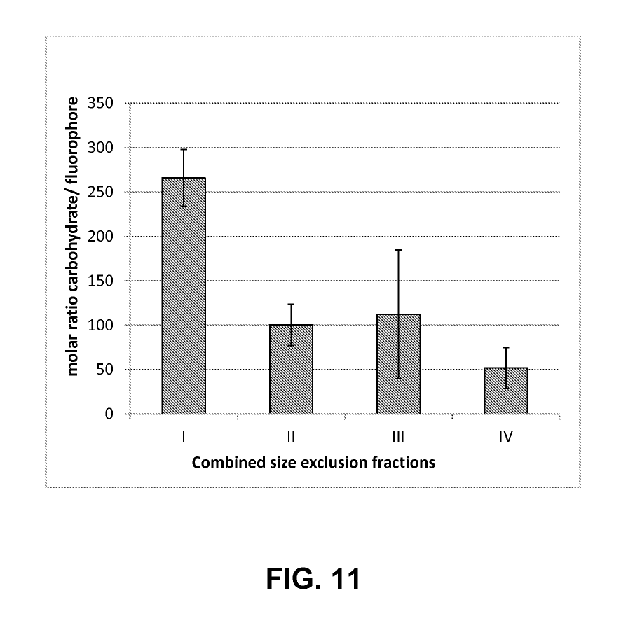

FIG. 11 shows the molar ratio of reducing sugar to fluorophore for the pooled chromatography fractions indicated. The molar ratio dropped sharply, by approximately 2.5-fold between pools 1 and 2, and then dropped by approximately 2-fold between pools 2 and 4. These data again indicate that the molecular weight of the xyloglucan had decreased with increasing incorporation of FITC-XGO.

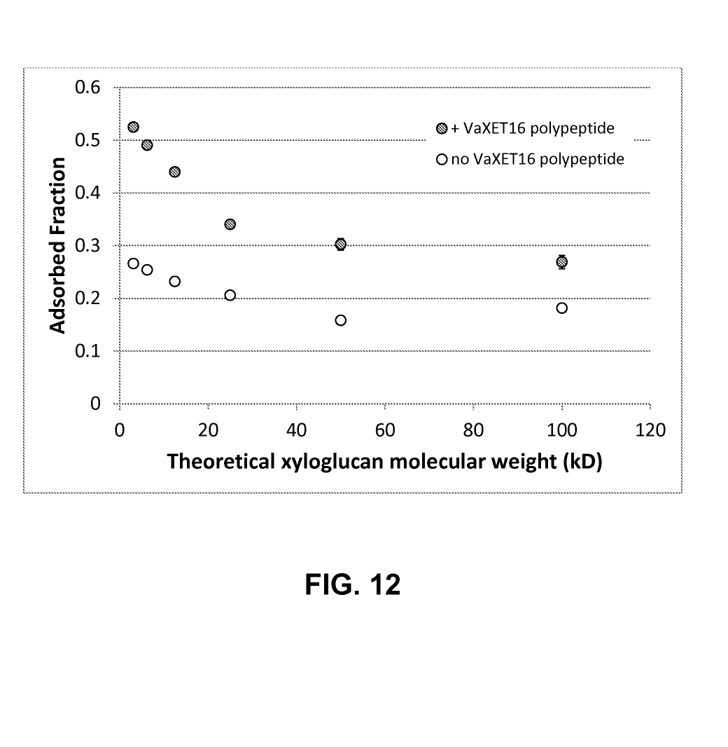

FIG. 12 shows the fraction of each theoretical FITC-XG bound to PET under the experimental binding conditions, both with and without VaXET16. For each molecular weight of FITC-XG, substantially more FITC-XG was bound when VaXET16 was present.

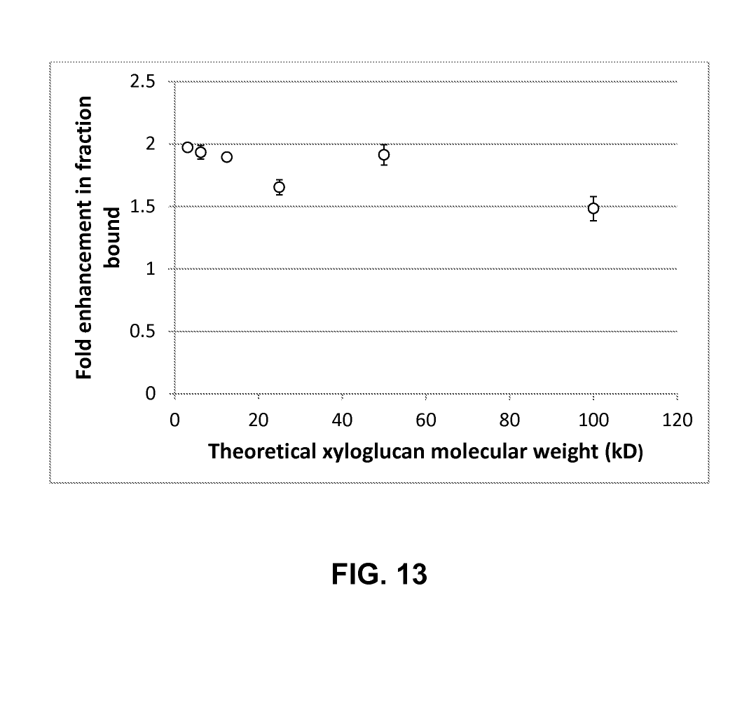

FIG. 13 shows the fold-enhancement of the fraction of FITC-XG bound to PET when VaXET16 was present over the fraction of FITC-XG bound when VaXET16 was absent.

### Example 14

### Xyloglucan Endotransglycosylase-Mediated Xyloglucan-Polyethylene Terephthalate Binding does not Occur Via Ternary Complex Formation

The binding or adsorption of VaXET16 to PET was assessed by incubating 1 μM VaXET16 in 20 mM phosphate pH 7.0 and then evaluating the concentration of VaXET16 in the supernatant by SDS-PAGE. Binding reactions (500 μl) were performed in microcentrifuge tubes wrapped in aluminum foil at 25° C. for up to 120 hours with shaking at 150 rpm in an INNOVA® shaker incubator. The binding reactions contained the following components: 1 μM VaXET16 with or without 30 discs of PET (approximately 60 mg per ml), and with or without 1 mg of FITC-XG per ml of 20 mM phosphate pH 7.0. A control incubation containing 1 mg FITC-XG per ml 20 mM phosphate pH 7.0 was performed under similar conditions. At 0, 2, 20, and 120 hours of incubation, 8 μl of each reaction supernatant was sampled, diluted 1:1 in sample buffer (Bio-Rad Laboratories, Inc., Hercules, Calif., USA) and run on an 8-16% CRITERION® TGX® stain-free SDS-PAGE gel (Bio-Rad Laboratories, Inc., Hercules, Calif., USA) at 300 volts for 20 minutes. Gels were visualized and bands quantified using ImageLab™ Software Version 3.0 (Bio-Rad Laboratories, Inc., Hercules, Calif., USA).

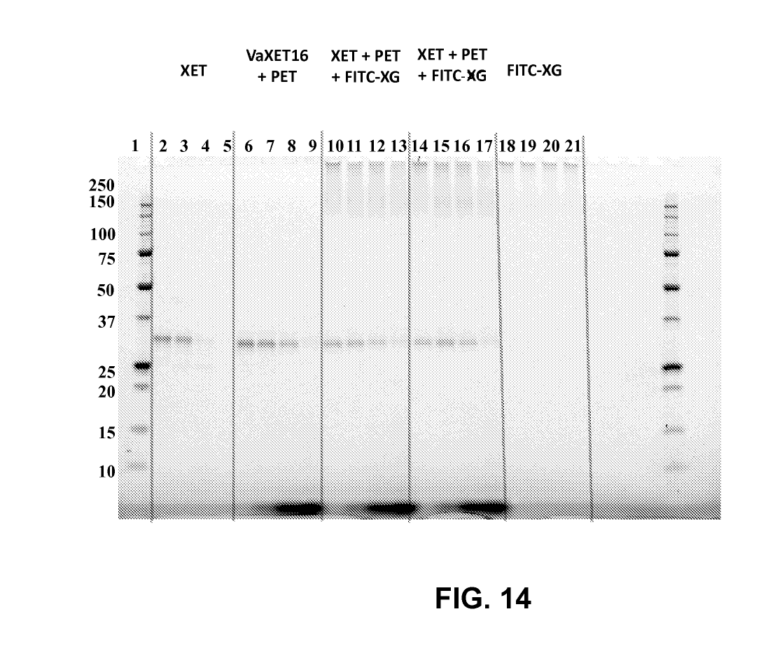

FIG. 14 shows the SDS-PAGE gel of the supernatants of PET-binding reactions sampled at various times. Lanes 2, 6, 10, 14, 18: 0 hours; lanes 3, 7, 11, 15, 19: 2 hours; lanes 4, 8, 12, 16, 20: 20 hours; and lanes 5, 9, 13, 17, 21: 120 hours. At the short incubation times, the supernatant contained equivalent concentrations of VaXET16, regardless of the presence of PET, indicating that no binding had occurred. At longer incubation times, more VaXET16 was present in the supernatants containing PET than those not containing PET. Since the band intensities with and without PET decayed to zero at longer incubation times, perhaps due to background protease activity, the concentration in solution could only be estimated. At 120 hours, the intensity of the VaXET16 polypeptide band was zero, assuming 100% of the protein had been removed from solution due to binding rather than proteolysis; the maximum concentration that could be potentially adsorbed to the PET was 1 μM. Under these reaction conditions at 120 hours of incubation, approximately 46%, or 0.46 mg per ml FITC-XG was bound to PET. As this represented a 4.5-fold molar excess of bound xyloglucan to the maximum amount of VaXET16 that could possibly be associated with PET, and since the actual loss of VaXET16 from solution was likely the result of proteolysis rather than binding, a direct ternary complex between PET, FITC-XG, and VaXET16 was conclusively excluded.

### Example 15

### Protease Digestion of Vigna angularis Xyloglucan Endotransglycosylase 16 does not Release FITC-XG from Polyethylene Terephthalate but Xyloglucanase Addition does Release FITC-XG from Polyethylene Terephthalate

Binding reactions were performed as described in Example 11, with the following exceptions. Triplicate 500 μl reactions were performed in foil-wrapped, 1.1 ml, 96-deep well plates (Axygen, Union City, Calif., USA). At 0, 20, 45, 97, 145, 169, and 175 hours of incubation, the plate was centrifuged at 3000 rpm (˜2200×g) for 1 minute, followed by transfer of 100 μl of each supernatant to a Costar 9017 flat bottomed, 96-well microtiter plate and the fluorescence was measured as described in Example 11. The 100 μl sample was then added back to the reaction plate. The plate was sealed and returned to the incubator. After 97 hours of incubation, trypsin (Sigma Aldrich, St. Louis, Mo., USA) was added to the reaction at a final concentration of 80 nM. The plate was sealed and incubated at 37° C. with shaking at 150 rpm overnight in an INNOVA® 40 shaker incubator. Following measurement of fluorescence at 145 hours of incubation, 0.1 mg of CELLIC® CTec3 cellulase (Novozymes A/S, Bagsvaerd, Denmark) per ml was added to the reaction. The plate was resealed and incubated at 50° C. overnight.

Following measurement of fluorescence at 169 hours of incubation, the PET sheets were washed with 1 ml of deionized water 3 times, mixing thoroughly by pipetting up and down during each wash, and were dried at 100° C. for 2 hours. The PET sheets were then transferred to a fresh 1.1 ml, 96-deep well plate and incubated with 0.05 mg of xyloglucanase per ml of 50 mM citrate pH 5.5 for 6 hours at 40° C. The pH was adjusted to 7.0, a 100 μl aliquot of the supernatant was removed, and the fluorescence was measured.

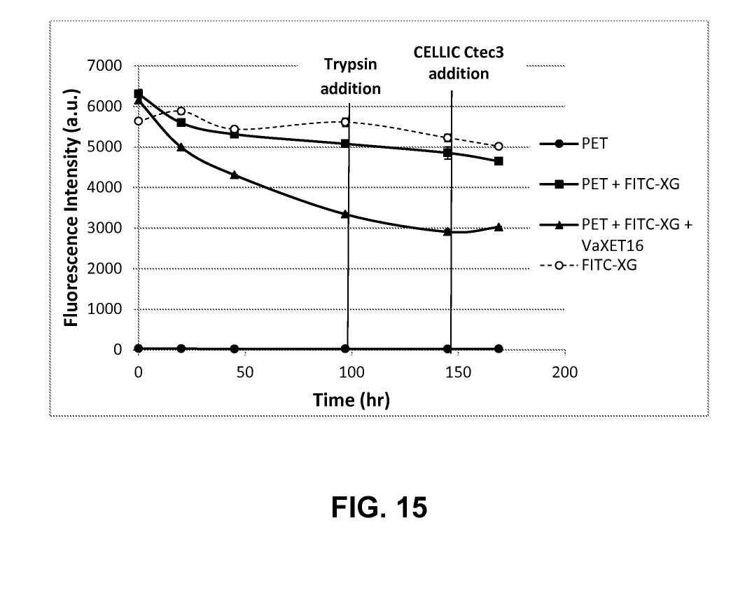

FIG. 15 shows the fluorescence intensity of the binding reactions as a function of time. The times, at which trypsin and cellulase were added, are indicated. From FIG. 15, it was clear that addition of trypsin did not release fluorescence from the PET sheets, indicating that VaXET16 was not forming a protein-link between xyloglucan and PET. It was also clear that addition of xyloglucanase did release some fluorescence from the PET, though the amount was relatively small; approximately 10% of the total adsorbed fluorescence was regained. The results indicate that hydrolysis of the accessible xyloglucan could be achieved by enzymatic hydrolysis. The results further indicate that functionalized xyloglucan and synthetic fabrics functionalized via association with functionalized xyloglucan could be returned to unfunctionalized form via hydrolysis of the associated xyloglucan, at least in part.

### Example 16

### Binding of Fluorescein Isothiocyanate-Labeled Xyloglucan to Nylon and Enhancement of Nylon-Xyloglucan Binding by Vigna angularis Xyloglucan Endotransglycosylase 16 and Arabidopsis thaliana Endotransglycosylase 14

Fluorescein isothiocyanate-labeled xyloglucan (FITC-XG) binding reactions were performed as described in Example 11 with the following exceptions: 1 strip, approximately 0.01 g of white tulle nylon fabric, was used instead of polyethylene terephthalate. Confocal microscopy was performed as described in Example 12 with the following exceptions: nylon samples were applied to the microscope slides, and images were obtained with photomultiplier tube voltage and offset set to maximize the image contrast. A series of images were obtained using equivalent photomultiplier tube settings; voltage of 499 and offset of 60, to allow semi-quantitative comparison of fluorescence intensities.

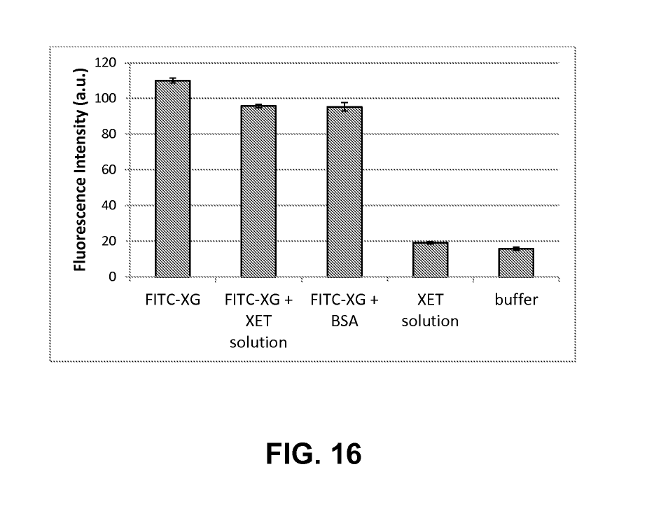

FIG. 16 shows the fluorescence intensity in solution for the FITC-XG solution incubated with nylon. In the presence of XET solution, a substantial reduction in fluorescence intensity in solution was observed, indicating that the xyloglucan had bound to the nylon.

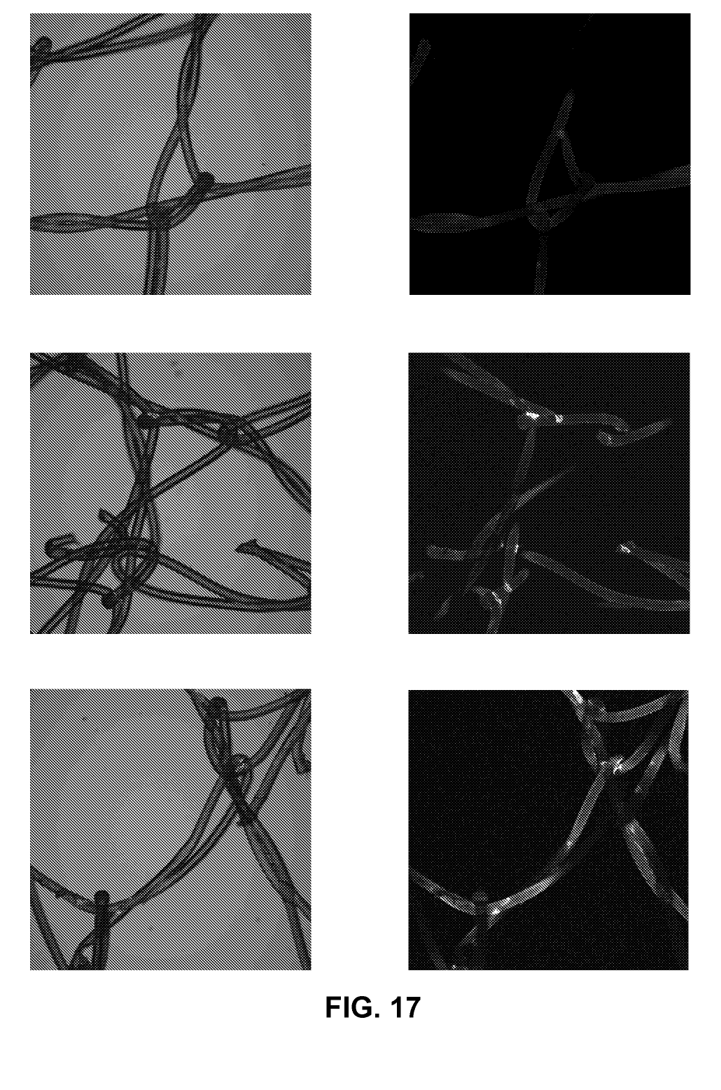

FIG. 17 shows overlaid transmission and fluorescence confocal microscopy images (on left) and fluorescence emission images (on right) of nylon incubated without FITC-XG (top panels); with FITC-XG and then extensively washed (middle panels); or with FITC-XG and XET solution (bottom panels). Nylon incubated with FITC-XG, either with or without XET solution, showed intense fluorescence in the hooks and loops of the nylon fibers, whereas nylon incubated with no FITC-XG did not show the fluorescent spots.

### Example 17

### Effect of Heat-Inactivated Vigna angularis Xyloglucan Endotransglycosylase 16 on Enhancing Binding of Fluorescein Isothiocyanate-Labeled Xyloglucan (FITC-XG) to Polyethylene Terephthalate

Confirmation that enzymatic activity of VaXET16 is required for binding of fluorescein isothiocyanate-labeled xyloglucan (FITC-XG) to polyethylene terephthalate (PET) was obtained in the following manner. Binding of FITC-XG was established by incubation of 1 mg/ml FITC-XG with 30 discs of PET interfacing fabric (Example 12) with or without 1 μM VaXET16 or with 1 μM VaXET16 that had previously been heat-inactivated by incubation at 95° C. for 25 minutes, in 500 μl of 25 mM phosphate buffer at pH 7.0 for 90 hours at room temperature in sealed 1.1 ml 1.1 ml 96-deep well plates (Axygen, Union City, Calif., USA). As controls, FITC-XG was incubated without PET discs, and PET was incubated without FITC-XG under otherwise identical conditions. At 90 hours of incubation, the supernatant fluorescence was measured as described in Example 11, with the exception that a SPECTRAMAX® Gemini XS (Molecular Devices, Sunnyvale, Calif., USA) was used. The supernatants were added back to the incubation well and either 0.1 mg of CELLIC CTec® (Novozymes, A/S, Bagsvaerd, Denmark) per ml or an additional 1 μM VaXET16 was then added to each incubation. Samples were incubated for an additional 48 hours at room temperature and the fluorescence of the supernatant was measured as described.

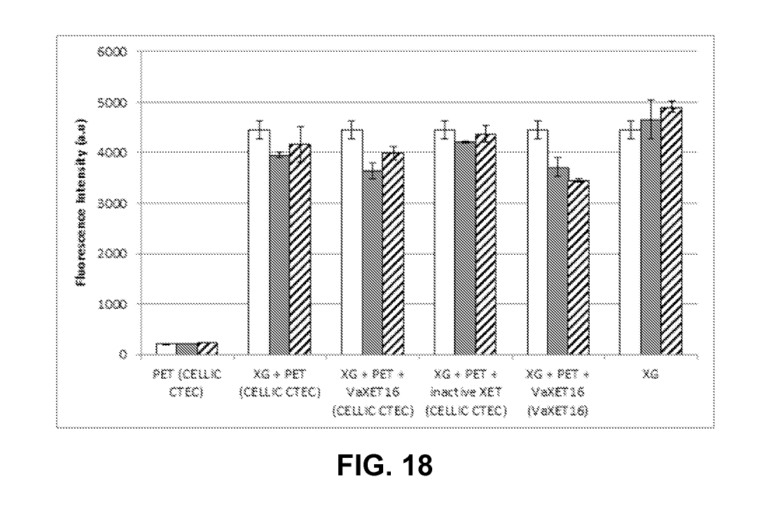

FIG. 18 shows the fluorescence intensity of the supernatants of the various incubations at time 0 (white); at 90 hours of incubation (gray); and 48 hours after addition of CELLIC CTec® or additional VaXET16. The enzyme or enzyme composition that was added at 90 hours is indicated in parentheses.

From FIG. 18, it is clear that from time 0 to 90 hours, fluorescence intensity of the FITC-XG in the supernatant decreased in samples containing PET as the FITC-XG bound to PET and the solution concentration concomitantly decreased. The decrease in intensity was 56% larger for samples that contain VaXET16 (780±244 a.u.) than that observed for samples that did not contain XET (501±185 a.u.) and 2.4-fold greater than the sample that contained heat-inactivated XET (239±178 a.u.). The results indicate that XET activity was required for enhanced PET binding by XG. In each case, when CELLIC CTec® was added to the incubations containing both FITC-XG and PET, fluorescence in solution increased. For the samples containing PET, this increase was attributed to hydrolysis of XG associated with PET, causing release of the FITC. Conversely, when additional VaXET16 was added to the incubation containing FITC-XG, PET, and VaXET16, the fluorescence in solution continued to decrease as additional XG was bound to the PET in an enzyme-dependent manner.

### Example 18

### Xyloglucan and Xyloglucan Endotransglycosylase-Mediated Binding of Silicon Dioxide to Multi-Fiber Fabrics

Binding of fluorescently-labeled silica (FITC-silicon oxide; Corpuscular Inc., Cold Spring, N.Y., USA) to multi-fiber test fabric (Kimble Chase Life Science and Research Products LLC) was assessed using the following protocol. Multi-fiber fabric was cut into strips of approximately 1 centimeter width. In Costar #3524, 48-well cell culture plates (Corning, Tewksbury, Mass., USA), the multi-fabric strips were rolled up and added to 1.5 ml solutions containing 20 mM phosphate buffer pH 7 with or without 2.5% (w/v) fluorescent silicon oxide, with or without 1.25 mg/ml tamarind seed xyloglucan (Megazyme, Ireland), with or without 6 μg/ml xyloglucan oligomers, and with or without 0.3 μM VaXET16. The plates were wrapped in foil and incubated for 4 hours in a 25° C. INNOVA® 40 shaker incubator with shaking at 220 rpm.

Following incubation, the multi-fiber strips were removed from the wells and rinsed under deionized water for approximately 30 seconds. The multi-fabric strips were then placed in new Costar #3524, 48-well cell culture plates and incubated with 1.5 ml 20 mM phosphate buffer pH 7 for 72 hours.

The multi-fabric strips were analyzed for fluorescence using Gel Doc™ EZ Imager (Bio-Rad Laboratories, Inc., Hercules, Calif., USA) with Image Lab version 3.0 software (Bio-Rad Laboratories, Inc., Hercules, Calif., USA) on SYBR® Green settings. The multi-fiber strips were exposed for 0.02 seconds and a TIFF image was obtained. Intensity histograms for each fabric were determined using ImageJ 1.47n software (National Institutes of Health, USA).

The multi-fabric test strips were then washed in 10 ml deionized water containing 20 μl Tide® for sensitive skin (Procter and Gamble, USA) for 30 minutes at 37° C. in 15 ml Corning® polypropylene centrifuge tubes (Corning, Tewksbury, Mass., USA). After washing, the strips were rinsed extensively under deionized water. The strips were analyzed for fluorescence as described.

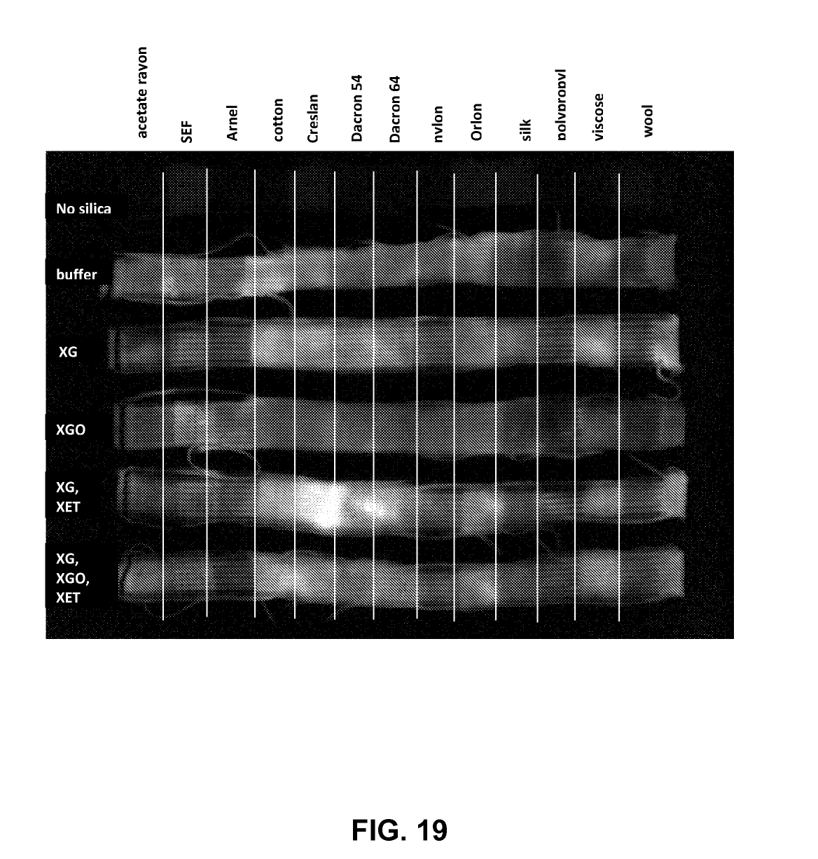

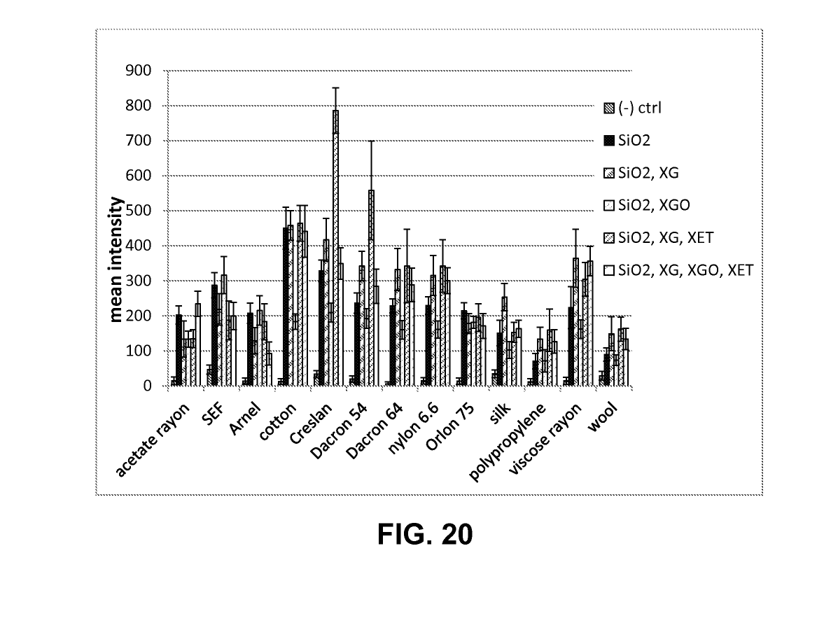

FIG. 19 shows the fluorescence image of the variously treated test fabrics aligned in a row; the treatments and the component fabrics are indicated. FIG. 20 shows the mean intensity for each fabric treatment prior to washing in detergent. In the absence of the fluorescent silica, the test fabric showed almost no intensity, and the test fabric was barely seen in the fluorescent image. In the presence of the FITC-SiO₂, fluorescence intensity was observed on the horizontal fibers of the test fabric incubated under each condition. Several fabrics additionally showed strong fluorescence when the SiO₂ was incubated with xyloglucan or particularly xyloglucan and VaXET16, including acrylic (Creslan), polyethylene terephthlate (Dacron 54 and 64), nylon 6.6, silk, viscose rayon, and wool. The presence of xyloglucan oligomers did not facilitate the association, and the addition of xyloglucan oligomers to xyloglucan and VaXET16 reduced the amount of association, potentially by reducing the degree of polymerization of xyloglucan in a VaXET16-dependent manner.

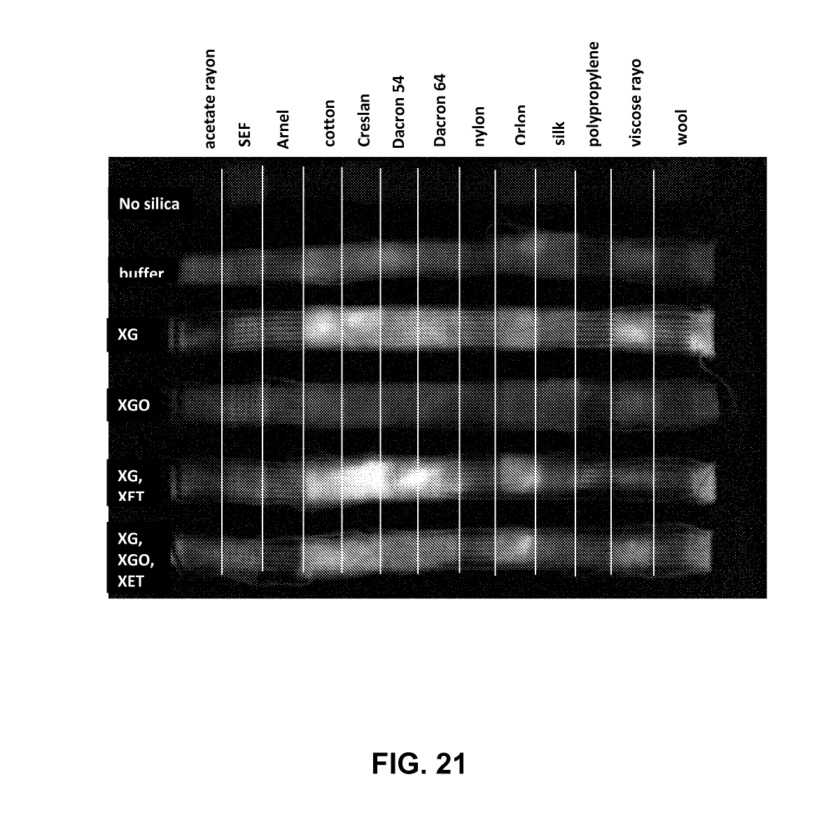

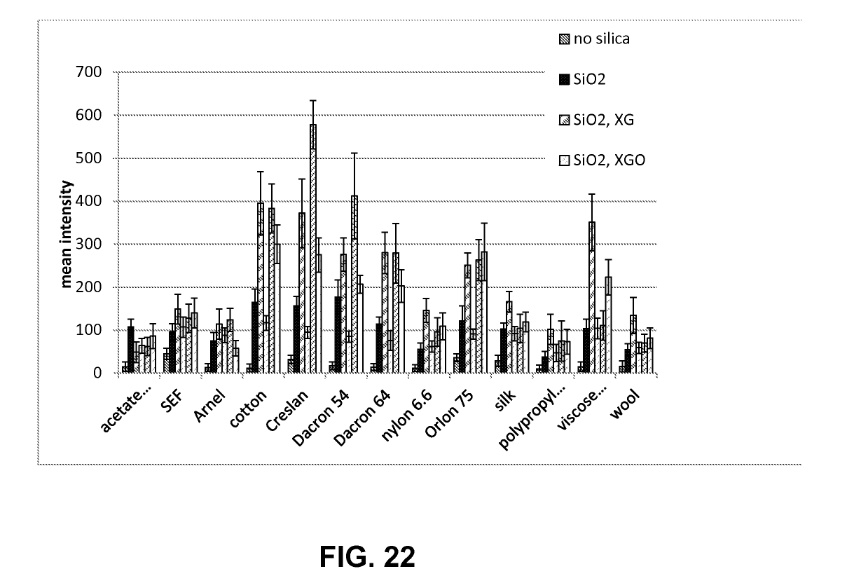

FIG. 21 shows the fluorescence image of the variously treated test fabrics following additional washing with a standard laundry detergent; the treatments and the component fabrics are indicated. FIG. 22 shows the mean intensity for each fabric treatment following washing in detergent. For most of the fabrics that had been incubated with FITC-SiO₂, the mean fluorescence intensities decreased following laundering, indicating that the detergent had washed FITC-SiO₂ from the test fabrics. The amount of SiO₂ fluorescence associated with the fabric was enhanced by xyloglucan or xyloglucan and VaXET16 for cotton, acrylic (Creslan), polyethylene terephthalate (Dacron 54 and 64), nylon 6.6, acrylic (Orlon 75), silk, polypropylene, viscose rayon, and wool. Acrylic and polyethylene terephthalate showed the largest VaXET16-dependent enhancement of the association. These data indicate that xyloglucan and particularly xyloglucan with VaXET16 can functionalize a variety of non-cellulosic fabrics with SiO₂.

### Example 19

### Binding of Fluorescein Isothiocyanate-Labeled Xyloglucan to Various Fibers in a Multi-Fiber Test Fabric

Binding of fluorescein isothiocyanate-labeled xyloglucan (FITC-XG) to multi-fiber fabric (Kimble Chase Life Science and Research Products, Vineland, N.J., USA) was assessed according to the following protocol. Multi-fiber fabric was cut into strips of approximately 1 centimeter width. In Costar #3524, 48-well cell culture plates, the multi-fabric strips, which were rolled and added to 1.5 ml solutions containing 20 mM phosphate buffer pH 7 with or without 1.25 g/I FITC-XG, with or without 12 mg/I FITC-XGO, and with or without 0.4 μM VaXET16. The plates were wrapped in foil and incubated for 72 hours in a 25° C. INNOVA® 40 shaker incubator with shaking at 220 rpm.

Following incubation, the multi-fabric strips were removed from the wells and extensively rinsed under running deionized water.

The multi-fabric strips were analyzed for fluorescence using a Gel Doc™ EZ Imager with Image Lab version 3.0 on SYBR® Green settings. The multi-fabric strips were exposed for 0.5 seconds and a TIFF image was obtained. Intensity histograms for each fabric were determined using ImageJ 1.47n software.

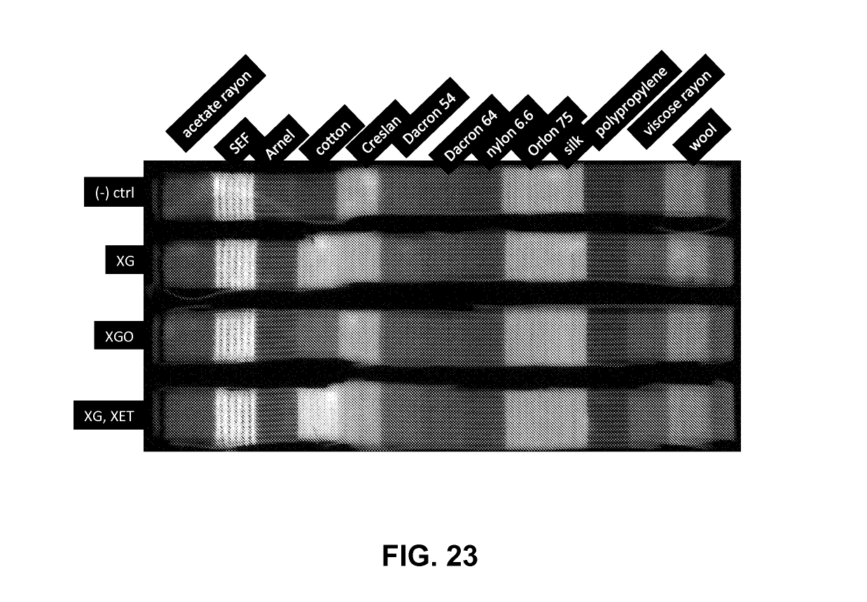

FIG. 23 shows a fluorescence image of the variously incubated multi-fabric strips. Comparing the control panels with the multi-fabric strips incubated with FITC-XG or FITC-XG and VaXET16, increased fluorescence intensity was observed for cotton, acrylic (Creslan), polyethylene terephthalate (Dacron), nylon 6.6, viscose rayon, and wool, indicating that these fibers had been functionalized with the FITC-XG. Comparing the cotton panels, it is clear that substantially more FITC-XG bound to cotton in the presence of VaXET16 than in the absence. Increases in FITC-XG binding to polyethylene terephthalate (Dacron), nylon, and viscose rayon were also observed in the presence of VaXET16. Comparing FITC-XG with FITC-XGO, it was apparent that for fabrics to which FITC-XG bound, much less of the short FITC-XGO bound than did the longer FITC-XG. These data indicate that a number of non-cellulosic textile materials can be functionalized with FITC-XG or xyloglucan in the presence of xyloglucan endotransglycosylase.

The present invention is further described by the following numbered paragraphs:

[1] A method for modifying a non-cellulosic textile material comprising treating the non-cellulosic textile material with a composition selected from the group consisting of:

(a) a composition comprising a xyloglucan endotransglycosylase, a polymeric xyloglucan, and a functionalized xyloglucan oligomer comprising a chemical group;

(b) a composition comprising a xyloglucan endotransglycosylase, a polymeric xyloglucan functionalized with a chemical group, and a functionalized xyloglucan oligomer comprising a chemical group;

(c) a composition comprising a xyloglucan endotransglycosylase, a polymeric xyloglucan functionalized with a chemical group, and a xyloglucan oligomer;

(d) a composition comprising a xyloglucan endotransglycosylase, a polymeric xyloglucan, and a xyloglucan oligomer;

(e) a composition comprising a xyloglucan endotransglycosylase and a polymeric xyloglucan functionalized with a chemical group;

(f) a composition comprising a xyloglucan endotransglycosylase and a polymeric xyloglucan;

(g) a composition comprising a xyloglucan endotransglycosylase and a functionalized xyloglucan oligomer comprising a chemical group; and

(h) a composition comprising a xyloglucan endotransglycosylase and a xyloglucan oligomer, under conditions leading to a modified non-cellulosic textile material, wherein the modified non-cellulosic textile material possesses a textile improvement compared to the unmodified non-cellulosic textile material.

[2] The method of paragraph 1, wherein the non-cellulosic textile material is selected from the group consisting of acetate, acrylic, nylon, olefin, polyester, rayon, spandex, lastex, and mixtures thereof.

[3] The method of paragraph 1 or 2, wherein the textile improvement is one or more improvements selected from the group consisting of anti-backstaining, anti-pilling, anti-shrinkage, anti-wear, anti-wrinkle, improved color appearance, fabric softness, improved shape retention, improved static control, anti-odor, anti-UV, water-repellency, anti-microbial, improved association with cellulosic textile in textile blends, and improved tensile strength.

[4] The method of any of paragraphs 1-3, wherein the average molecular weight of the polymeric xyloglucan or the polymeric xyloglucan functionalized with a chemical group ranges from 2 kDa to about 500 kDa.

[5] The method of any of paragraphs 1-4, wherein the average molecular weight of the xyloglucan oligomer or the functionalized xyloglucan oligomer comprising a chemical group ranges from 0.5 kDa to about 500 kDa

[6] The method of any of paragraphs 1-5, wherein the xyloglucan endotransglycosylase is present at a concentration of about 0.1 nM to about 1 mM.

[7] The method of any of paragraphs 1-6, wherein the polymeric xyloglucan or the polymeric xyloglucan functionalized with a chemical group is present at about 1 mg per g of the non-cellulosic textile material to about 1 g per g of the non-cellulosic textile material.

[8] The method of any of paragraphs 1-7, wherein the xyloglucan oligomer or the functionalized xyloglucan oligomer is present with the polymeric xyloglucan or the polymeric xyloglucan functionalized with a chemical group at about 50:1 molar ratio to about 0.5:1 xyloglucan oligomer or functionalized xyloglucan oligomer to polymeric xyloglucan.

[9] The method of any of paragraphs 1-8, wherein the concentration of the polymeric xyloglucan, the polymeric xyloglucan functionalized with a chemical group, the xyloglucan oligomer, or the functionalized xyloglucan oligomer comprising a chemical group incorporated into the material is about 0.01 g to about 500 mg per g of the non-cellulosic textile material.

[12] The method of any of paragraphs 1-9, wherein the chemical group is a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

[11] The method of any of paragraphs 1-10, wherein the xyloglucan endotransglycosylase is obtainable from a plant or microorganism.

[12] The method of paragraph 11, wherein the plant is selected from the group consisting of a dicotyledon and a monocotyledon.

[13] The method of paragraph 12, wherein the dicotyledon is selected from the group consisting of azuki beans, cauliflowers, cotton, poplar or hybrid aspen, potatoes, rapes, soy beans, sunflowers, thalecress, tobacco, and tomatoes.

[14] The method of paragraph 13, wherein the monocotyledon is selected from the group consisting of wheat, rice, corn and sugar cane.

[15] The method of any of paragraphs 1-14, wherein the xyloglucan endotransglycosylase is produced by aerobic cultivation of a transformed host organism containing the appropriate genetic information from a plant.

[16] The method of any of paragraphs 1-15, wherein the composition is a detergent composition.

[17] The method of any of paragraphs 1-15, wherein the composition is a fabric care composition.

[18] The method of any of paragraphs 1-15, wherein the treating of the non-cellulosic textile material is a prewash step.

[19] The method of any of paragraphs 1-15, wherein the treating of the non-cellulosic textile material is a wash step.

[20] The method of any of paragraphs 16-19, wherein the composition further comprises one or more components selected from the group consisting of surfactants, builders, bleaching agents, dye transfer inhibiting agents, chelants, dispersants, polysaccharides, softening agents, suds suppressors, carriers, enzymes, enzyme stabilizing systems, polyacids, soil removal agents, anti-redeposition agents, hydrotropes, opacifiers, antioxidants, bactericides, dyes, perfumes, brighteners and mixtures thereof.

[21] The method of paragraph 20, wherein the enzymes are selected from the group consisting of amylases, cellulases, cutinases, lipases, oxidases, peroxidases, proteases, xyloglucanases, and combinations thereof.

[22] The method of paragraphs 1-21, wherein the non-cellulosic textile material is subsequently unfunctionalized by addition of xyloglucanase, endo-β-1-4 glucanase, cellulase, or combinations thereof.

[23] The method of paragraph 22, wherein the subsequently unfunctionalized non-cellulosic textile material is refunctionalized by addition of polymeric xyloglucan functionalized with a chemical group or functionalized xyloglucan oligomers and xyloglucan endotransglycosylase.

[24] A modified non-cellulosic textile material obtained by the method of any of paragraphs 1-23.

[25] A modified non-cellulosic textile material comprising (a) a polymeric xyloglucan and a functionalized xyloglucan oligomer comprising a chemical group; (b) a polymeric xyloglucan functionalized with a chemical group and a functionalized xyloglucan oligomer comprising a chemical group; (c) a polymeric xyloglucan functionalized with a chemical group, and a xyloglucan oligomer; (d) a polymeric xyloglucan, and a xyloglucan oligomer; (e) a polymeric xyloglucan functionalized with a chemical group; (f) a polymeric xyloglucan; (g) a functionalized xyloglucan oligomer comprising a chemical group; or (h) a xyloglucan oligomer.

[26] A composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; and (h) a composition comprising a xyloglucan endotransglycosylase and a xyloglucan oligomer.

[27] A detergent or fabric care composition for non-cellulosic textile materials comprising a surfactant and (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

[28] A detergent additive for non-cellulosic textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

[29] A textile coating or finishing composition for non-cellulosic textile materials comprising (a) a xyloglucan endotransglycosylase, a polymeric xyloglucan, and a functionalized xyloglucan oligomer comprising a chemical group; (b) a xyloglucan endotransglycosylase, a polymeric xyloglucan functionalized with a chemical group, and a functionalized xyloglucan oligomer comprising a chemical group; (c) a xyloglucan endotransglycosylase, a polymeric xyloglucan functionalized with a chemical group, and a xyloglucan oligomer; (d) a xyloglucan endotransglycosylase, a polymeric xyloglucan, and a xyloglucan oligomer; (e) a xyloglucan endotransglycosylase and a polymeric xyloglucan functionalized with a chemical group; (f) a xyloglucan endotransglycosylase and a polymeric xyloglucan; (g) a xyloglucan endotransglycosylase and a functionalized xyloglucan oligomer comprising a chemical group; or (h) a xyloglucan endotransglycosylase and a xyloglucan oligomer.

The inventions described and claimed herein are not to be limited in scope by the specific aspects herein disclosed, since these aspects are intended as illustrations of several aspects of the invention. Any equivalent aspects are intended to be within the scope of the inventions. Indeed, various modifications of the inventions in addition to those shown and described herein will become apparent to those skilled in the art from the foregoing description. Such modifications are also intended to fall within the scope of the appended claims. In the case of conflict, the present disclosure including definitions will control.

1. A method for modifying a non-cellulosic textile material comprising treating the non-cellulosic textile material with a composition selected from the group consisting of:
(a) a composition comprising a xyloglucan endotransglycosylase, a polymeric xyloglucan, and a functionalized xyloglucan oligomer comprising a chemical group;
(b) a composition comprising a xyloglucan endotransglycosylase, a polymeric xyloglucan functionalized with a chemical group, and a functionalized xyloglucan oligomer comprising a chemical group;
(c) a composition comprising a xyloglucan endotransglycosylase, a polymeric xyloglucan functionalized with a chemical group, and a xyloglucan oligomer;
(d) a composition comprising a xyloglucan endotransglycosylase, a polymeric xyloglucan, and a xyloglucan oligomer;
(e) a composition comprising a xyloglucan endotransglycosylase and a polymeric xyloglucan functionalized with a chemical group;
(f) a composition comprising a xyloglucan endotransglycosylase and a polymeric xyloglucan;
(g) a composition comprising a xyloglucan endotransglycosylase and a functionalized xyloglucan oligomer comprising a chemical group; and
(h) a composition comprising a xyloglucan endotransglycosylase and a xyloglucan oligomer, under conditions leading to a modified non-cellulosic textile material, wherein the modified non-cellulosic textile material possesses a textile improvement compared to the unmodified non-cellulosic textile material.

2. The method of claim 1, wherein the non-cellulosic textile material is selected from the group consisting of acetate, acrylic, nylon, olefin, polyester, rayon, spandex, lastex, and mixtures thereof.

3. The method of claim 1, wherein the textile improvement is one or more improvements selected from the group consisting of anti-backstaining, anti-pilling, anti-shrinkage, anti-wear, anti-wrinkle, improved color appearance, fabric softness, improved shape retention, improved static control, anti-odor, anti-UV, water-repellency, anti-microbial, improved association with cellulosic textile in textile blends, and improved tensile strength.

4. The method of claim 1, wherein the average molecular weight of the polymeric xyloglucan or the polymeric xyloglucan functionalized with a chemical group ranges from 2 kDa to about 500 kDa.

5. The method of claim 1, wherein the average molecular weight of the xyloglucan oligomer or the functionalized xyloglucan oligomer comprising a chemical group ranges from 0.5 kDa to about 500 kDa

6. The method of claim 1, wherein the xyloglucan endotransglycosylase is present at a concentration of about 0.1 nM to about 1 mM.

7. The method of claim 1, wherein the polymeric xyloglucan or the polymeric xyloglucan functionalized with a chemical group is present at about 1 mg per g of the non-cellulosic textile material to about 1 g per g of the non-cellulosic textile material.

8. The method of claim 1, wherein the xyloglucan oligomer or the functionalized xyloglucan oligomer is present with the polymeric xyloglucan or the polymeric xyloglucan functionalized with a chemical group at about 50:1 molar ratio to about 0.5:1 xyloglucan oligomer or functionalized xyloglucan oligomer to polymeric xyloglucan.

9. The method of claim 1, wherein the concentration of the polymeric xyloglucan, the polymeric xyloglucan functionalized with a chemical group, the xyloglucan oligomer, or the functionalized xyloglucan oligomer comprising a chemical group incorporated into the material is about 0.01 g to about 500 mg per g of the non-cellulosic textile material.

10. The method of claim 1, wherein the chemical group is a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

11. The method of claim 1, wherein the composition is a detergent composition or a fabric care composition.

12. The method of claim 1, wherein the treating of the non-cellulosic textile material is a prewash step or a wash step.

13. The method of claim 11, wherein the composition further comprises one or more components selected from the group consisting of surfactants, builders, bleaching agents, dye transfer inhibiting agents, chelants, dispersants, polysaccharides, softening agents, suds suppressors, carriers, enzymes, enzyme stabilizing systems, polyacids, soil removal agents, anti-redeposition agents, hydrotropes, opacifiers, antioxidants, bactericides, dyes, perfumes, brighteners and mixtures thereof.

14. The method of claim 13, wherein the enzymes are selected from the group consisting of amylases, cellulases, cutinases, lipases, oxidases, peroxidases, proteases, xyloglucanases, and combinations thereof.

15. The method of claim 1, wherein the non-cellulosic textile material is subsequently unfunctionalized by addition of xyloglucanase, endo-β-1-4 glucanase, cellulase, or combinations thereof.

16. The method of claim 15, wherein the subsequently unfunctionalized non-cellulosic textile material is refunctionalized by addition of polymeric xyloglucan functionalized with a chemical group or functionalized xyloglucan oligomers and xyloglucan endotransglycosylase.

17-21. (canceled)


SEQUENCE LISTING (22 sequences)

SEQ ID NO: 1 - DNA, 879 bp, Vigna angularis
atgggttcttctttgtggacttgtctgattctgttatcactggcttctgcttctttcgctgccaacccaagaactccaattgatgtaccatttggcagaaactatgtgcctacttgggcctttgatcatatcaaatatctcaatggaggttctgagattcagcttcatctcgataagtacactggtactggattccagtccaaagggtcatacttgtttggtcacttcagcatgtacataaaattggttcctggtgattcagctggcacagtcactgctttctatttatcgtccacaaacgcagaacatgatgaaatagacttcgagttcttgggaaacagaactgggcaaccatacattttacaaacaaatgtgttcaccggaggcaaaggtgacagagagcagagaatctacctctggtttgaccctacgactcaataccacagatattcagtgctatggaacatgtaccagattgtattctatgtggatgactacccaataagggtgttcaagaacagcaatgacttgggagtgaagttccccttcaatcaaccaatgaaaatatacaacagtttgtggaatgcagatgactgggctacaaggggtggtttggagaaaacagattggtccaaagcccccttcatagcctcttacaagggcttccacattgatgggtgtgaggcctcagtgaatgccaagttctgtgacacacaaggcaagaggtggtgggatcaaccagagtttcgtgaccttgatgctgctcagtggcaaaaactggcttgggtacgcaacaaatacaccatctacaactactgcactgatcgcaaacgctactctcaagtccctccagagtgcaccagagaccgtgacatttaa

SEQ ID NO: 2 - DNA, 876 bp, Vigna angularis
atgggctcgtccctctggacttgtttgatcctcctctccttggcatcggcatccttcgcagcgaaccctcgaactccgatcgatgtgcctttcggacggaactacgtgccgacatgggcattcgaccacattaagtatttgaacggaggctcggagatccagttgcatctcgacaagtacaccggcactggtttccagtcgaagggctcctacttgttcggacatttctccatgtacatcaaattggtgcctggtgactcggcaggaactgtcaccgcattctacctctcgtcgacaaacgcagagcatgacgaaatcgacttcgagttcctcggcaacaggacaggacagccgtacatcctccagaccaacgtcttcacaggaggcaaaggtgatcgggaacagcggatctacttgtggttcgatcccacaacccagtaccataggtactcggtgctctggaacatgtatcagatcgtcttctacgtcgacgattatccgatccgagtgttcaagaactccaacgacttgggcgtcaaattccccttcaaccagcccatgaagatttacaactcgttgtggaacgccgacgattgggcaaccaggggtggtctcgagaagacagattggtcgaaagcacctttcatcgcgtcgtacaagggtttccacatcgacggatgtgaagcctccgtgaacgccaagttctgtgacacccagggcaaacgatggtgggatcagccggaattccgggatttggatgcagcccagtggcagaagctcgcgtgggtcaggaacaagtacaccatctataactactgtaccgatcggaaacgatattcgcaggtgcctcccgagtgtacacgcgatagggacatc

SEQ ID NO: 3 - protein, 292 aa, Vigna angularis
MGSSLWTCLILLSLASASFAANPRTPIDVPFGRNYVPTWAFDHIKYLNGGSEIQLHLDKYTGTGFQSKGSYLFGHFSMYIKLVPGDSAGTVTAFYLSSTNAEHDEIDFEFLGNRTGQPYILQTNVFTGGKGDREQRIYLWFDPTTQYHRYSVLWNMYQIVFYVDDYPIRVFKNSNDLGVKFPFNQPMKIYNSLWNADDWATRGGLEKTDWSKAPFIASYKGFHIDGCEASVNAKFCDTQGKRWWDQPEFRDLDAAQWQKLAWVRNKYTIYNYCTDRKRYSQVPPECTRDRDI

SEQ ID NO: 4 - DNA, 864 bp, Arabidopsis thaliana
atggcgtgtttcgcaaccaaacagcctctgttgttgtctctcctccttgccattggcttctttgtggtggctgcatctgccggaaacttctatgagagctttgatatcacttggggtaatggtcgtgccaacatattcgagaatggacagcttctcacttgtactcttgacaaggtctccggctcaggttttcaatccaagaaggagtacttgtttggtaagatcgacatgaagctcaagcttgtcgctggaaactctgctggcaccgtcaccgcctactacctatcgtcaaaaggcacggcatgggatgagattgacttcgagtttttgggaaatcgcacaggacatccttacactatccacaccaatgtgttcaccggaggtaaaggcgaccgtgagatgcagttccgtctctggttcgatcccactgcggatttccacacctacaccgtccactggaaccctgttaacatcatcttccttgtggatgggatcccaattcgggtgttcaagaacaacgagaaaaatggggtggcttaccctaagaaccagccgatgaggatatactcaagcctttgggaagccgatgactgggctacagaaggcggtcgcgtgaagatcgactggagcaacgcaccattcaaggcctcttacagaaacttcaacgaccaaagctcatgcagcaggacatcaagctcaaaatgggtgacttgcgagccaaacagcaactcgtggatgtggacgactctcaatcctgcccagtacggaaaaatgatgtgggtgcaacgagacttcatgatctacaactattgtaccgattttaagagattccctcaaggcctccccaaggagtgtaaactttga

SEQ ID NO: 5 - DNA, 861 bp, Arabidopsis thaliana
atggcctgtttcgcaaccaaacagccgttgttgctctccttgttgctcgccatcggtttcttcgtggtggcagcctccgcaggaaacttctatgagtccttcgacatcacctggggcaacggaagggcgaacattttcgaaaacggtcagctcctcacttgtacgctcgacaaggtgtccggctccggtttccagtcgaagaaggagtacttgttcggcaagatcgacatgaagctcaagttggtggcaggtaactcggcaggtaccgtcacagcgtactatttgtcgtccaagggaactgcgtgggacgaaatcgacttcgagttcctcggcaaccgtacaggacacccctacactattcacaccaacgtcttcaccggaggcaagggtgatcgggagatgcagttcaggctctggttcgacccgacagcggatttccatacttacacggtgcattggaaccccgtcaacatcattttcctcgtcgacggaatccccatccgagtcttcaagaacaacgagaagaacggtgtggcgtatcccaaaaaccagccgatgcgcatctactcctcgttgtgggaagcggacgactgggccacagaaggcggacgcgtcaagatcgactggtcgaacgcaccgttcaaggcgtcgtaccggaacttcaacgaccagtcgtcctgttcgaggacttcgtcgtccaagtgggtcacctgtgaacccaactcgaactcgtggatgtggactactctcaaccctgcccagtacggcaagatgatgtgggtgcagagggacttcatgatctacaactattgtaccgatttcaaacgattccctcagggtctccccaaggaatgtaaactc

SEQ ID NO: 6 - protein, 287 aa, Arabidopsis thaliana
MACFATKQPLLLSLLLAIGFFVVAASAGNFYESFDITWGNGRANIFENGQLLTCTLDKVSGSGFQSKKEYLFGKIDMKLKLVAGNSAGTVTAYYLSSKGTAWDEIDFEFLGNRTGHPYTIHTNVFTGGKGDREMQFRLWFDPTADFHTYTVHWNPVNIIFLVDGIPIRVFKNNEKNGVAYPKNQPMRIYSSLWEADDWATEGGRVKIDWSNAPFKASYRNFNDQSSCSRTSSSKWVTCEPNSNSWMWTTLNPAQYGKMMWVQRDFMIYNYCTDFKRFPQGLPKECKL

SEQ ID NO: 7 - DNA, 50 bp, Artificial Sequence
  ARTIFICIAL DNA PRIMER
ttcctcaatcctctatatacacaactggccatgggctcgtccctctggac

SEQ ID NO: 8 - DNA, 48 bp, Artificial Sequence
  ARTIFICIAL DNA PRIMER
tgtcagtcacctctagttaattagatgtccctatcgcgtgtacactcg

SEQ ID NO: 9 - DNA, 29 bp, Artificial Sequence
  Artificial DNA Primer
taattaactagaggtgactgacacctggc

SEQ ID NO: 10 - DNA, 31 bp, Artificial Sequence
  Artificial DNA Primer
catggccagttgtgtatatagaggattgagg

SEQ ID NO: 11 - DNA, 36 bp, Artificial Sequence
  Artificial DNA Primer
acatgtctttgataagctagcgggccgcatcatgta

SEQ ID NO: 12 - DNA, 36 bp, Artificial Sequence
  Artificial DNA Primer
tacatgatgcggcccgctagcttatcaaagacatgt

SEQ ID NO: 13 - DNA, 41 bp, Artificial Sequence
  Artificial DNA Primer
ttaatcgccttgcagcacaccgcttcctcgctcactgactc

SEQ ID NO: 14 - DNA, 47 bp, Artificial Sequence
  Artificial DNA Primer
acaataaccctgataaatgcggaacaacactcaaccctatctcggtc

SEQ ID NO: 15 - DNA, 53 bp, Artificial Sequence
  Artificial DNA Primer
agatagggttgagtgttgttccgcatttatcagggttattgtctcatgagcgg

SEQ ID NO: 16 - DNA, 42 bp, Artificial Sequence
  Artificial DNA Primer
ttctacacgaaggaaagaggaggagagagttgaacctggacg

SEQ ID NO: 17 - DNA, 47 bp, Artificial Sequence
  Artificial DNA Primer
aggttcaactctctcctcctctttccttcgtgtagaagaccagacag

SEQ ID NO: 18 - DNA, 43 bp, Artificial Sequence
  Artificial DNA Primer
tcagtgagcgaggaagcggtgtgctgcaaggcgattaagttgg

SEQ ID NO: 19 - DNA, 54 bp, Artificial Sequence
  Artificial DNA Primer
ttcctcaatcctctatatacacaactggccatggcctgtttcgcaaccaaacag

SEQ ID NO: 20 - DNA, 47 bp, Artificial Sequence
  Artificial DNA Primer
agctcgctagagtcgacctagagtttacattccttggggagaccctg

SEQ ID NO: 21 - DNA, 27 bp, Artificial Sequence
  Artificial DNA Primer
taggtcgactctagcgagctcgagatc

SEQ ID NO: 22 - DNA, 40 bp, Artificial Sequence
  Artificial DNA Primer
catggccagttgtgtatatagaggattgaggaaggaagag